(19) United States
(12) Patent Application Publication
(10) Pub. No.: US 2004/0259875 A1
(43) Pub. Date: Dec. 23, 2004

(54) Amine derivatives

(76) Inventors: Takeshi Yura, Nara-ken (JP); Muneto Mogi, Nara-ken (JP); Yuka Ikegami, Kyoto-fu (JP); Tsutomu Masuda, Aichi-ken (JP); Toshio Kokubo, Nara-ken (JP); Klaus Urbahns, Hyogo-ken (JP); Timothy B Lowinger, Wuppertal (DE); Nagahiro Yoshida, Kyoto-fu (JP); Joachim Freitag, Munchen (DE); Heinrich Meier, Wuppertal (DE); Reilinde Nopper, Grenzach-Whylen (DE); Makiko Marumo, Nara-ken (JP); Masahiro Shiroo, Cambridge (GB); Masaomi Tajimi, Kyoto-fu (JP); Keisuke Takeshita, Kyoto-fu (JP); Toshiya Moriwaki, Nara-ken (JP); Yasuhiro Tsukimi, Hyogo-ken (JP)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/485,481

(22) PCT Filed: Jul. 31, 2002
(86) PCT No.: PCT/EP02/08493

(30) Foreign Application Priority Data
Jul. 31, 2001 (JP) 232503-2001
Dec. 25, 2001 (JP) 392310-2001

(51) Int. Cl. C07D 333/38; C07D 215/38; A61K 31/537; A61K 31/381; A61K 31/425; A61K 31/165

(52) U.S. Cl. 514/237.5; 514/313; 514/378; 514/447; 514/618; 544/162; 546/159; 549/71; 564/161

(57) ABSTRACT

An amine derivative, its tautomeric or stereoisomeric form, or a salt thereof which has vanilloid receptor 1 (VR1) antagonistic activity, is disclosed. The amine derivative has an excellent activity as VR1 antagonist and useful for the prophylaxis and treatment of diseases associated with VR1 activity, in particular for the treatment of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

### TECHNICAL FIELD

The present invention relates to an amine derivative, which is useful as an active ingredient of pharmaceutical preparations. The amine derivatives of the present invention have vanilloid receptor 1 (VR1) antagonistic activity, and can be used for the prophylaxis and treatment of diseases associated with VR1 activity, in particular for the treatment of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

### BACKGROUND ART

Vanilloid compounds are characterized by the presence of vanillyl group or a functionally equivalent group. Examples of several vanilloid compounds or vanilloid receptor modulators are vanillin (4-hydroxy-3-methoxy-benzaldehyde), guaiacol (2-methoxy-phenol), zingerone (4-/4-hydroxy-3-methoxyphenyl/-2-butanon), eugenol (2-methoxy4-/2-propenyl/phenol), and capsaicin (8-methy-N-vanillyl-6-noneneamide).

Among others, capsaicin, the main pungent ingredient in “hot” chili peppers, is a specific neurotoxin that desensitizes C-fiber afferent neurons. Capsaicin and its analogues, such as resiniferatoxin, are shown to be effective in the treatment of urological disorder e.g., urinary incontinence and overactive bladder, due to the desensitization of C-fiber afferent neurons [(Michael B Chancellor and William C. de Groat, The Journal of Urology Vol. 162, 3-11, 1999) and (K. E. Andersson et al., BJU International, 84, 923-947, 1999)]. However, the mechanism in which capsaicin and other analogues cause the desensitization of C-fiber afferent neurons is very complicated.

Vanilloid receptor (VR) is a specific neuronal membrane recognition site for capsaicin. It is expressed almost exclusively by primary sensory neurons involved in nociception and neurogenic inflammation. The VR functions as a cation-selective ion channel with a preference for calcium. Capsaicin interacts with VR1, which is a functional subtype of the VR and predominantly expressed in cell bodies of dorsal root ganglia (DRG) or nerve endings of afferent sensory fibers including C-fiber nerve endings [Tominaga M, Caterina M J, Malmberg A B, Rosen T A, Gilbert H, Skinner K, Raumann B E, Basbaum A I, Julius D: The cloned capsaicin receptor integrates multiple pain-producing stimuli. Neuron. 21: 531-543, 1998]. The VR1 was recently cloned [Caterina M J, Schumacher M A, Tominaga M, Rosen T A, Levine J D, Julius D: Nature 389: 816-824, (1997)] and identified as a nonselective cation channel with six transmembrane domains that is structurally related to the TRP (transient receptor potential) channel family. Binding of capsaicin to VR1 allows sodium, calcium and possibly potassium ions to flow down their concentration gradients, causing initial depolarization and release of neurotransmitters from the nerve terminals.

VR1 can therefore be viewed as a molecular integrator of chemical and physical stimuli that elicit neuronal signals in a pathological conditions or diseases.

There are abundant of direct or indirect evidence that shows the relation between VR1 activity and diseases such as pain, ischaemia, and inflammatory (e.g., WO 99/00115 and WO00/50387). Further, it has been demonstrated that VR1 transduce reflex signals that are involved in the overactive bladder of patients who have damaged or abnormal spinal reflex pathways [De Groat W C: A neurologic basis for the overactive bladder. Urology 50 (6A Suppl): 36-52, 1997]. Desensitisation of the afferent nerves by depleting neurotransmitters using VR1 agonists such as capsaicin has been shown to give promising results in the treatment of bladder dysfunction associated with spinal cord injury and multiple sclerosis [(Maggi C A: Therapeutic potential of capsaicin-like molecules—Studies in animals and humans. Life Sciences 51: 1777-1781, 1992) and (DeRidder D; Chandiramani V; Dasgupta P; VanPoppel H; Baert L; Fowler C J: Intravesical capsaicin as a treatment for refractory detrusor hyperreflexia: A dual center study with long-term follow-up. J. Urol. 158: 2087-2092, 1997)].

It is anticipated that antagonism of the VR1 would lead to the blockage of neurotransmitter release, resulting in prophylaxis and treatment of the condition and diseases associated with VR1 activity.

It is therefore expected that antagonists of the VR1 can be used for prophylaxis and treatment of the condition and diseases including urology disorder, chronic pain, neuropathic pain, postoperative pain, rheumatoid arthritic pain, neuralgia, neuropathies, algesia, nerve injury, ischaemia, neurodegeneration, stroke, incontinence and inflammatory disorders. “Urological disorder” used herein refers to e.g., urinary incontinence and overactive bladder. Urinary incontinence and overactive bladder encompass detrusor hyper-reflexia, detrusor instability and urgency/frequency syndrome, such as urge urinary incontinence and the like.

WO 00/50387 discloses the compounds having a vanilloid receptor agonist activity represented by the general formula:

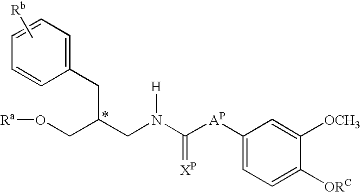

wherein;

X^{P} is an oxygen or sulfur atom;

A^{P} is —NHCH₂— or —CH₂—;

R^{a} is a substituted or unsubstituted C₁₋₄ alkyl group, or R^{a1}CO—;

wherein

R^{a1} is an alkyl group having 1 to 18 carbon atoms, an alkenyl group having 2 to 18 carbon atoms, or substituted or unsubstituted aryl group having 6 to 10 carbon atoms;

R^{b} is a hydrogen atom, an alkyl group having 1 to 6 carbon atoms, an alkoxy group having 1 to 6 carbon atoms, a haloalkyl group having 1 to 6 carbon atoms or a halogen atom;

R^{C} is a hydrogen atom, an alkyl group having 1 to 4 carbon atom, an aminoalkyl, a diacid monoester or α-alkyl acid; and

the asteric mark * indicates a chiral carbon atom, and their pharmaceutically acceptable salts.

WO 00/61581 discloses amine derivatives represented by the general formula:

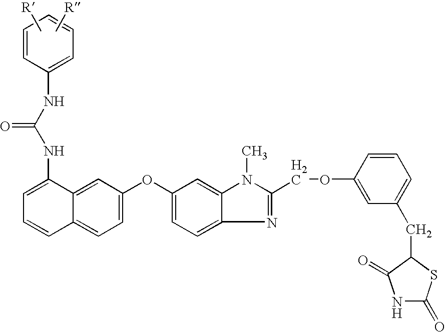

wherein

(R′, R″) represent (F, F), (CF₃, H), or (iPr, iPr)

as useful agents for diabetes, hyperlipemia, arteriosclerosis and cancer.

WO 00/75106 discloses the compounds represented by the general formula:

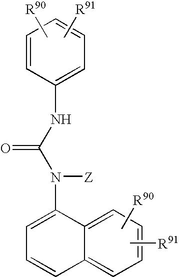

wherein

Z represents

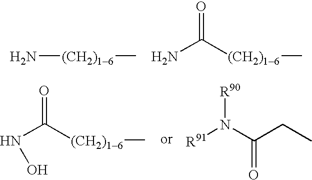

in which

R⁹⁰ is hydrogen, C₁₋₁₂ alkyl, C₃₋₈ cycloalkyl, or the like, and R⁹¹ is amino-C₁₋₆ alkyl, aminocarbonyl-C₁₋₆ alkyl, or hydroxyaminocarbonyl C₁₋₆ alkyl; and

R⁹⁰ and R⁹¹ are independently selected from the group consisting of H, C₁₋₆ alkyl, C₁₋₆ alkylthio, C₁₋₆ alkoxy, fluoro, chloro, bromo, iodo, and nitro;

as useful agents for treating MMP-mediated diseases in mammals.

However, none of these reference discloses simple phenyl-naphthyl urea derivatives having VR1 antagonistic activity.

The development of a compound, which has effective VR1 antagonistic activity and can be used for the prophylaxis and treatment of diseases associated with VR1 activity, in particular for the treatment of urology disorder including urinary incontinence and/or overactive bladder, has been desired.

### SUMMARY OF THE INVENTION

As the result of extensive studies on chemical modification of amine derivatives, the present inventors have found that the compound of novel chemical structure related to the present invention have unexpectedly excellent VR1 antagonistic activity. This invention is to provide the following general formula (I), its tautomeric or stereoisomeric form, and the salts thereof:

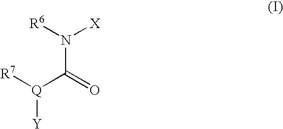

wherein

X represents C₃₋₈ cycloalkyl optionally fused by benzene, thienyl, thienyl C₁₋₆ straight alkyl, quinolyl, 1,2-oxazolyl substituted by R¹, naphthyl optionally substituted by R⁴ and R⁵, phenyl fused by C₄₋₈ cycloalkyl, phenyl fused by saturated C₄₋₈ heterocycle having one or two O atoms, carbazolyl of which N-H is substituted by N-R¹, phenyl fused by indanone, phenyl fised by indan, phenyl fused by cyclohexanone, phenyl fused by dihydrofuranone, phenyl substituted by R¹, R² and R³, phenyl C₁₋₆ straight alkyl of which phenyl is substituted by R¹, R² and R³, phenyl fused by unsaturated 5-6 membered hetero ring having one or two hetero atoms selected from the group consisting of N, O, S, and SO₂, wherein the hetero ring is optionally substituted by R¹,

wherein

R¹, R² and R³ are identical or different and represent hydrogen, halogen, straight-chain or branched C₁₋₆ alkyl, straight-chain or branched C₁₋₆ alkylcarbarnoyl, carbamoyl, straight-chain or branched C₁₋₆ alkoxy, carboxyl, nitro, amino, straight-chain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ aqlkylthio, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ alkyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, the substituent represented by the formula —SO₂—NH-R¹² (R¹² represents hydrogen, 5-methyl-isoxazole, or 2,4-dimethylpyrimidine) or

phenyl optionally substituted by one to three substituents,

wherein

the substituents are each identical or different and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;

R⁴ represents hydrogen, hydroxy, or straight-chain or branched C₁₋₆ alkoxy;

R⁵ represents hydrogen, hydroxy, or straight-chain or branched C₁₋₆ alkoxy;

Q represents CH or N;

R⁶ represents hydrogen or methyl;

R⁷ represents hydrogen or methyl; and

Y represents

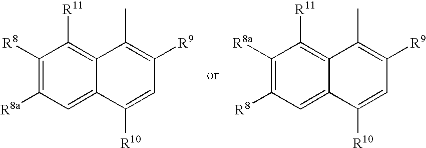

wherein

R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, C₁₋₆ alkylsulfonamino, or the group represented by the formula

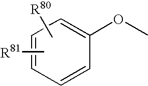

wherein

R⁸⁰ and R⁸¹ are each identical or different and represent hydrogen, halogen, or straight-chain or branched C₁₋₆ alkoxy;

R^{8a} represents hydrogen or halogen;

R⁹ and R¹¹ are each identical or different and represent hydrogen, halogen, or nitro; and

R¹⁰ represents hydrogen, halogen, carboxy, carbamoyl, cyano, or straight-chain or branched C₁₋₆ alkyl optionally substituted by the substituent, which substituent is selected from the group consisting of hydroxy, amino, di(straight-chain or branched C₁₋₆ alkyl)amino, piperidino, morpholino, and methylpiperazino.

The compounds of the present invention suprisingly show excellent VR1 antagonistic activity. They are, therefore, suitable especially as VR1 antagonists and in particular for the production of medicament or medical composition, which may be useful to treat urological disorder. Since the amine derivatives of the present invention antagonize VR1 activity, they are useful for treatment and prophylaxis of diseases as follows: urology disorder (e.g., urinary incontinence and overactive bladder), chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

In another embodiment, the amine derivative of the formula (I) is those wherein;

X represents

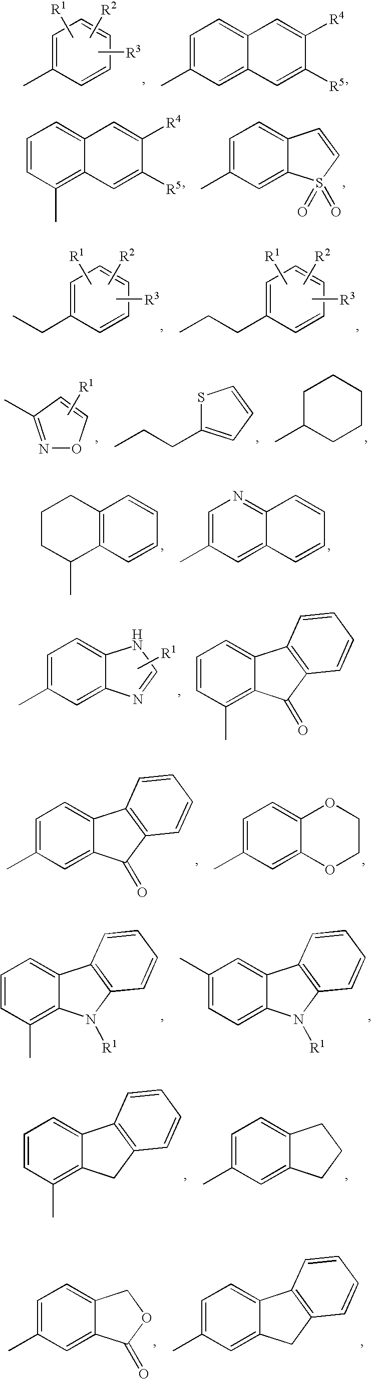

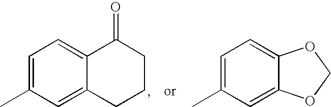

wherein

R¹, R² and R³ are different or identical and represent hydrogen, halogen, straight-chain or branched C₁₋₆ alkyl, straight-chain or branched C₁₋₆ alkylcarbamoyl, carbamoyl, straight-chain or branched C₁₋₆ alkoxy, carboxyl, nitro, amino, straight-chain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ alkylthio, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ alkyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, the substituent represented by the formula —SO₂—NH-R¹² (R¹² represents hydrogen, 5-methyl-isoxazole, or 2,4-dimethylpyrimidine) or

phenyl optionally substituted by one to three substituents,

wherein

the substituents are each different or identical and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;

R⁴ represents hydrogen, hydroxy, or straight-chain or branched C₁₋₆ alkoxy;

R⁵ represents hydrogen, hydroxy, or straight-chain or branched C₁₋₆ alkoxy;

Q represents CH or N;

R⁶ represents hydrogen or methyl;

R⁷ represents hydrogen or methyl; and

Y represents

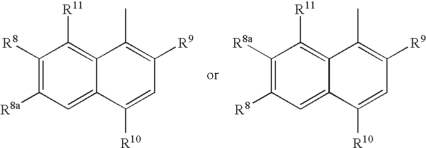

wherein

R⁸ represents hydroxy, straight-chain or branched C₁₋₆ alkoxy, straight-chain or branched C₁₋₆ alkanoyloxy, C₃₋₆ C₃₋₆ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, straight-chain or branched C₁₋₆ alkylsulfonamino, or the group represented by the formula

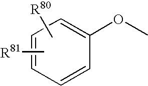

wherein

R⁸⁰ and R⁸¹ are each identical or different and represent hydrogen, halogen, or straight-chain or branched C₁₋₆ alkoxy;

R^{8a} represents hydrogen or halogen;

R⁹ represents hydrogen or halogen;

R¹⁰ represents hydrogen, halogen, or straight-chain or branched C₁₋₆ alkyl optionally substituted by hydroxy; and

R¹¹ represents hydrogen, halogen, or nitro

or a salt thereof.

In yet another embodiment, the amine derivative of the formula (I) is those wherein;

R⁶ represents hydrogen;

R⁷ represents hydrogen;

Y represents

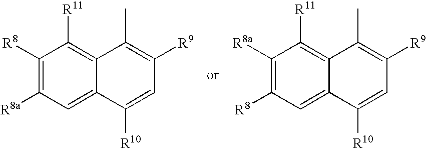

wherein

R⁸ represents hydroxy, straight-chain or branched C₁₋₆ alkoxy, straight-chain or branched C₁₋₆ alkanoyloxy, C₃₋₆ C₃₋₆ cycloalkyhmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or C₁₋₆ alkylsulfonamino;

R^{8a} represents hydrogen, chloro, or fluoro;

R⁹ represents hydrogen or halogen;

R¹⁰ represents hydrogen, halogen or straight-chain or branched C₁₋₆ alkyl optionally substituted by hydroxy; and

R¹¹ represents hydrogen or halogen;

or a salt thereof.

In yet another embodiment, the amine derivative of the formula (I) is those wherein;

R⁶ represents hydrogen;

R⁷ represents hydrogen;

Y represents

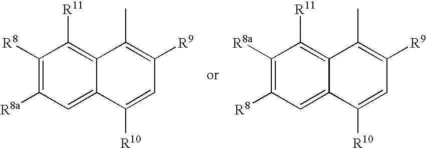

wherein

R⁸ represents hydroxy, straight-chain or branched C₁₋₆ alkoxy, straight-chain or branched C₁₋₆ alkanoyloxy, C₃₋₆ C₃₋₆ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or straight-chain or branched C₁₋₆ alkylsulfonamino;

R^{8a} represents hydrogen;

R⁹ represents hydrogen, bromo, chloro, or fluoro;

R¹⁰ represents hydrogen,halogen or straight-chain or branched C₁₋₆ alkyl optionally substituted by hydroxy; and

R¹¹ represents hydrogen, chloro, or fluoro

or a salt thereof.

In yet another embodiment, the amine derivative of the formula (I) is those wherein;

R⁶ represents hydrogen;

R⁷ represents hydrogen;

Y represents

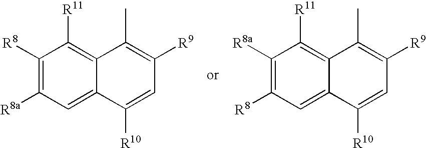

wherein

R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, or straight-chain or branched C₁₋₆ alkylamino;

R^{8a} represents hydrogen;

R⁹ represents bromo or chloro;

R¹⁰ represents bromo, chloro, or straight-chain or branched C₁₋₆ alkyl optionally substituted by hydroxy; and

R¹¹ represents hydrogen

or a salt thereof.

In yet another embodiment, the amine derivative of the formula (I) is those wherein;

R⁶ represents hydrogen;

R⁷ represents hydrogen;

Y represents

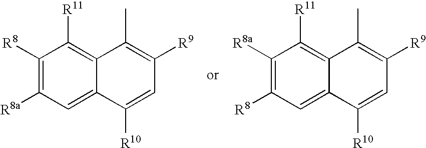

wherein

R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, or straight-chain or branched C₁₋₆ alkylamino;

R^{8a} represents hydrogen;

R⁹ represents chloro;

R¹⁰ represents chloro; and

R¹¹ represents hydrogen

or a salt thereof.

The present invention further provides the medicament having one of the compounds mentioned-above and one or more pharmaceutically acceptable excipients.

The compound of the formula (I) of the present invention can be, but not limited to be, prepared by the general methods [A]-[K] below. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3^{rd} Edition, John Wiley, New York, 1999)” by Greene and Wuts.

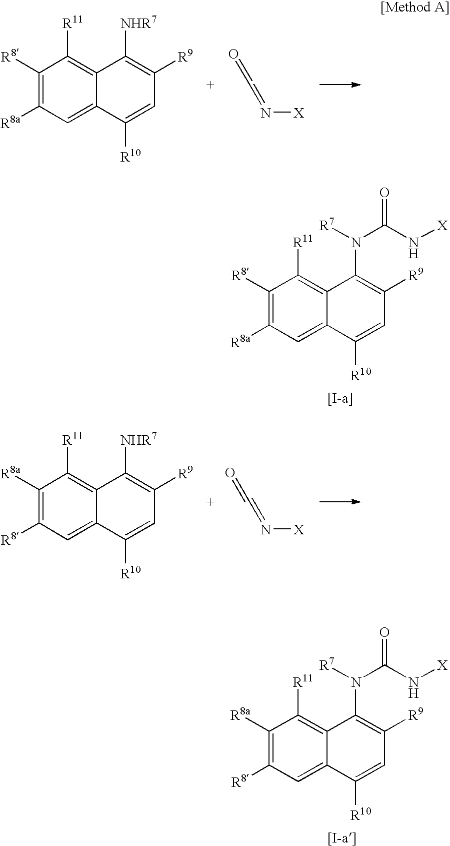

The compound [I-a] and the compound [I-a′], wherein R⁸, is hydroxy, strait-chain or branched C₁₋₆ alkoxy, strait-chain or branched C₁₋₆ alkoxy, benzoyloxy, straight-chain or branched strait-chain or branched C₁₋₆ alkenyloxy, C₃₋₈ cycloalkylmethoxy, phenyl C₁₋₆ alkylamino, straight-chain or branched C₁₋₆ alkylamino, or di(straight-chain or branched C₁₋₆ alkyl)amino and R⁷, R⁹, R¹⁰, R¹¹, and X are the same as defined above, can be prepared by the reaction of a substituted naphthylamine and isocyanate. The reaction may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

The substituted naphthylamine and isocyanate are commercially available or can be prepared by the use of known techniques.

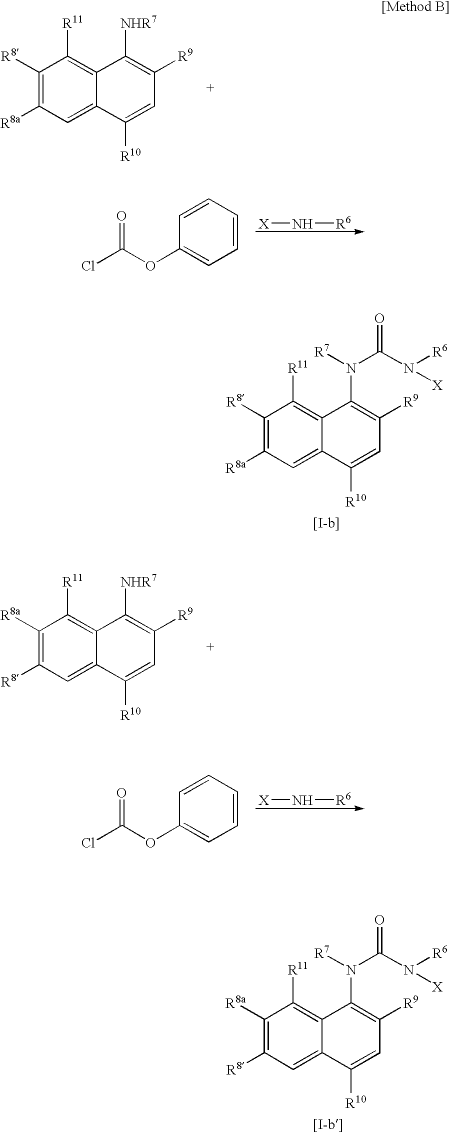

The compound [I-b] and the compound [1-b′], wherein R⁶, R⁷, R^{8a}, R⁸, R⁹, R¹⁰, R¹¹, and X are the same as defined above, can be prepared by (1) reacting a substituted naphthylamine and phenylchloroformate, and (2) adding amine represented by the formula X-NH-R⁶ (wherein R⁶ and X are the same as defined above) to the reaction mixture. The reaction (1) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitriles such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 20° C. to 50° C. The reaction may be conducted for, usually, 30 minutes to 10 hours and preferably 1 to 24 hours.

The reaction can be advantageously carried out in the presence of a base including, for instance, an alkali metal hydride such as sodium hydride and potassium hydride; alkali metal carbonates such as sodium carbonate and potassium carbonate; alkali metal hydrogen carbonates such as sodium hydrogen carbonate and potassium hydrogen carbonate; organic amines such as pyridine, triethylamine and N,N-diisopropylethylamine, and others.

The reaction (2) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitriles such as acetonitrile; amides such as N,N-dimethylformamide (DMF), N,N-dimethylacetamide and N-methylpyrrolidone; sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 120° C. The reaction may be conducted for, usually, 1 hour to 48 hours and preferably 2 to 24 hours.

The substituted naphthylamine, phenylchloroformate and amine are commercially available or can be prepared by the use of known techniques.

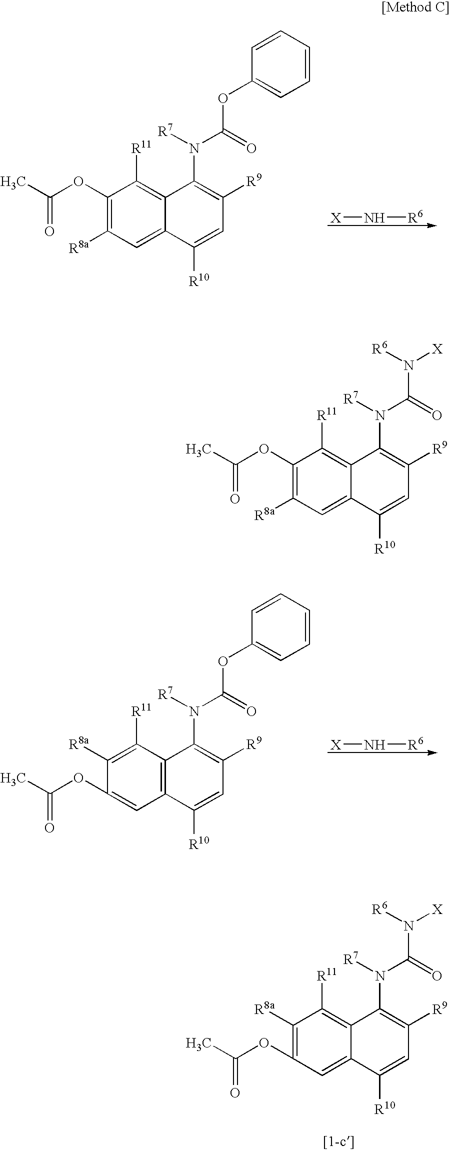

The compound [I-c] and the compound [1-c′], wherein R⁶, R⁷, R^{8a}, R⁹, R¹⁰, R¹¹, and X are the same as defined above, can be prepared by the reaction of a substituted naphthylamine carbamate and amine represented by the formula X-NH-R⁶ (wherein R⁶ and X are the same as defined above). The reaction may be carried out in a solvent including, for instance, halogenated hydrocarbons such as dichloromethane, chloroform and 1,2-dichioroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 120° C. The reaction may be conducted for, usually, 1 hour to 48 hours and preferably 2 to 24 hours.

The substituted naphthylamine carbamate and amine are commercially available or can be prepared by the use of known techniques.

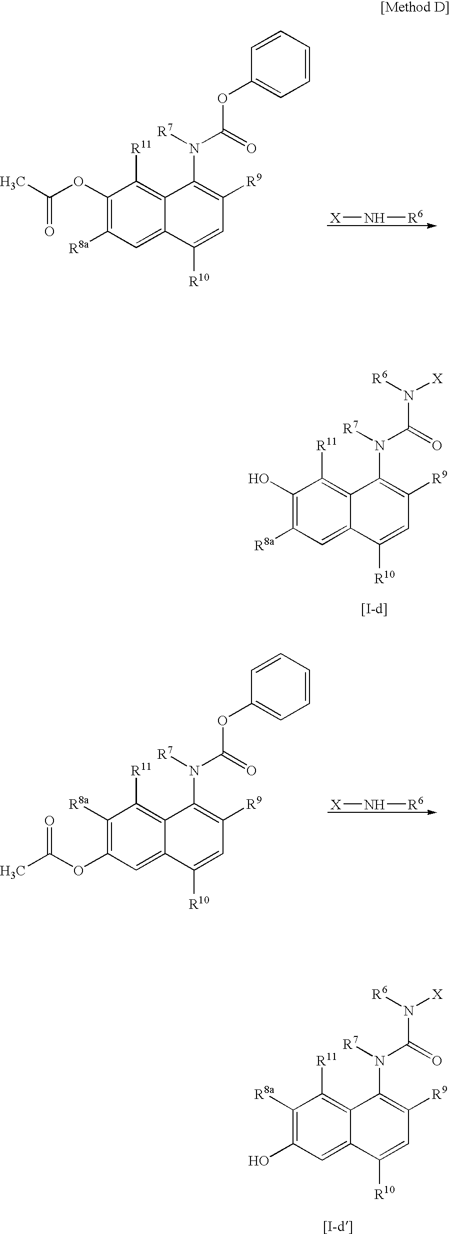

The compound [I-d] and the compound [I-d′], wherein R⁶, R⁷, R^{8a}, R⁹, R¹⁰, R¹¹, and X are the same as defined above, can be prepared by (1) reacting a substituted naphthylamine carbamate and amine represented by the formula X-NH-R⁶ (wherein R⁶ and X are the same as defined above), and (2) adding base to the reaction mixture. The reaction (1) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethaned aromatic hydrocarbons such as benzene, toluene and xylene; ketones such as acetone; nitriles such as acetonitrile, amides such as N,N-dimethylformamide (DMF), N,N-dimethylacetamide and N-methylpyrrolidone; sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 20° C. to 120° C. The reaction may be conducted for, usually, 1 hour to 48 hours and preferably 2 to 24 hours.

The reaction (2) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO); alcohol such as tert-butanol, methanol and ethanol; water, and others. Optionally, two or more of the solvents selected from the listed above can be mixed and used.

The reaction temperature can be optionally set depending on the compounds to be reacted. The reaction temperature is usually, but not limited to, about 30° C. to 100° C. The reaction may be conducted for, usually, 1 hour to 48 hours and preferably 2 to 24 hours.

The base used in the reaction (2) can be, for instance, alkali metal alkoxide such as sodium methoxide and sodium ethoxide; alkali metal hydroxide such as sodium hydroxide and potassium hydroxide, and others.

The substituted naphthylamine carbamate and amine are commercially available or can be prepared by the use of known techniques.

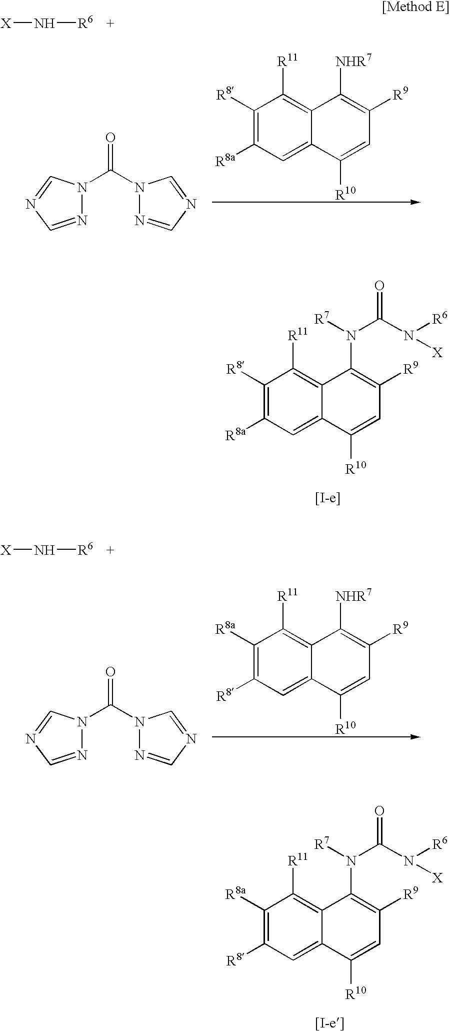

The compound [I-e] and the compound [I-e′], wherein R⁷, R^{8′}, R^{8a}, R⁹, R¹⁰, R¹¹, and X are the same as defined above, can be prepared by (1) reacting amine represented by the formula X-NH-R⁶ (wherein R⁶ and X are the same as defined above) and 1,1′-carbonyldi(1,2,4-triazole) (CDT) and (2) adding substituted naphthylamine to the reaction mixture. The reaction (1) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 40 hours and preferably 1 to 24 hours.

The reaction (2) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 30° C. to 100° C. The reaction may be conducted for, usually, 1 hour to 48 hours and preferably 2 to 24 hours. The amine, 1,1′-carbonyldi(1,2,4-triazole) (CDT) and substituted naphthylamine are commercially available or can be prepared by the use of known techniques.

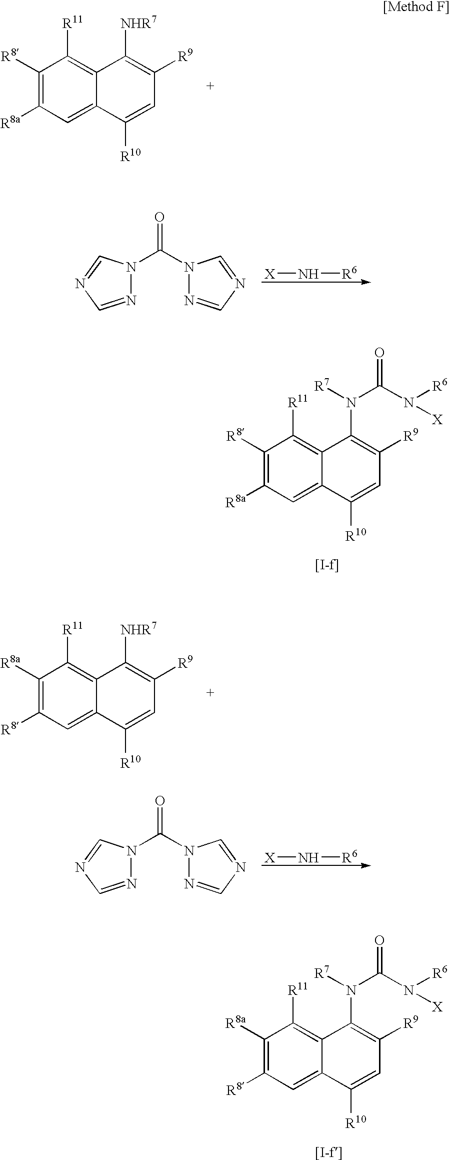

The compound [1-f] and the compound [1-f′], wherein R⁶, R⁷, R^{8′}R^{8a}, R⁹, R¹⁰, R¹¹ and X is the same as defined above, can be prepared by (1) reacting a substituted naphthylamine and 1,1′-carbonyldi(1,2,4-triazole) (CDT), and (2) adding amine represented by the formula X-NH-R⁶ (wherein R⁶ and X are the same as defined above) to the reaction mixture. The reaction (1) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on The reaction temperature can be optionally set depending on the compounds to be reacted. The reaction temperature is usually, but not limited to, about 20° C. to 100° C. The reaction may be conducted for, usually, 30 minutes to 10 hours and preferably 1 to 24 hours.

The reaction (2) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 1 hour to 48 hours and preferably 2 to 24 hours.

The substituted naphthylamine, 1,1′-carbonyldi(1,2,4-triazole) (CDT) and amine are commercially available or can be prepared by the use of known techniques.

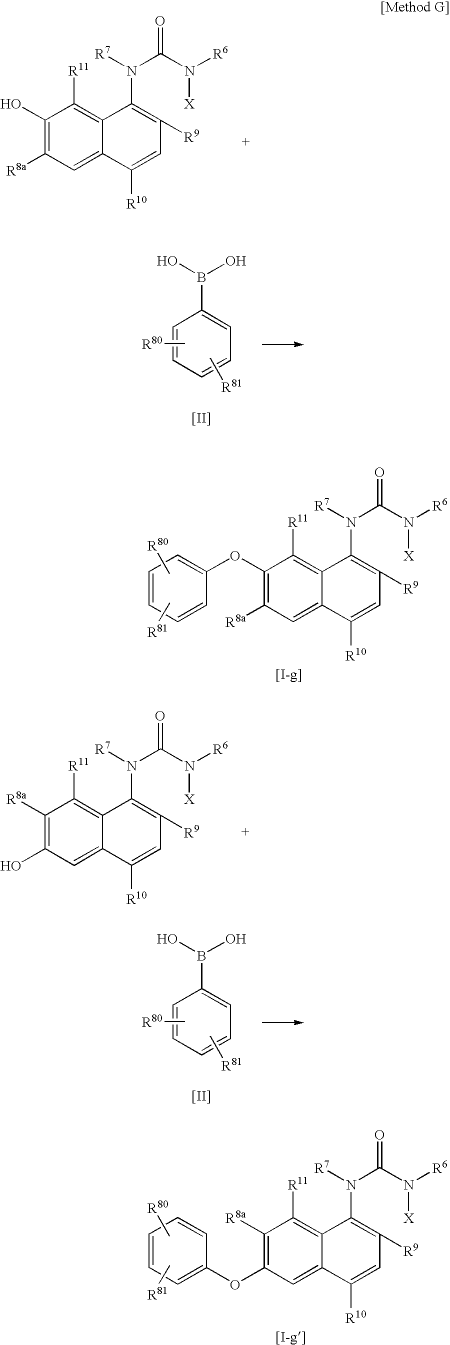

The compound [R-g] and compound [R-g′] wherein X, R⁶, R⁷, R⁹, R¹⁰, and R¹¹ are the same as defined above and; R⁸⁰ and R⁸¹ are identical or different and represent hydrogen, halogen, or C₁₋₆ alkoxy, can be, but not limited to be, prepared by reacting substituted naphthyl amine with an arylboronic acid [II], wherein R⁸⁰ and R⁸¹ are the same as defined above.

The reaction may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 40 hours and preferably 1 to 24 hours.

The reaction can be advantageously conducted in the presence of substance having catalytic activity. Such substances include, but not limited to, copper salts, such as copper (II) acetate, or the like.

The reaction can also be advantageously carried out in the presence of a base including, for instance, organic amines such as triethylamine and N,N-diisopropylethylamine, and the others.

The arylboronic acid and coper salts are commercially available or can be prepared by the use of known techniques.

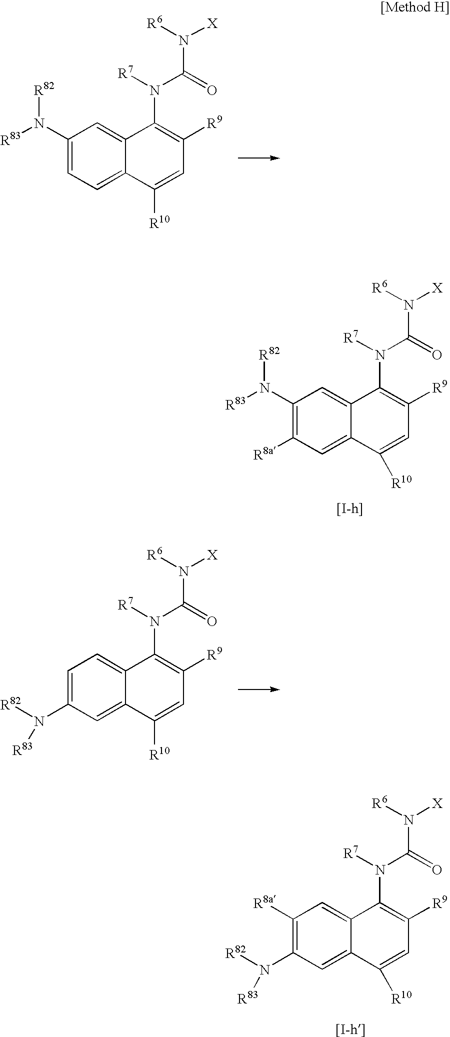

The compound [I-h] and the compound [1-h′], wherein R⁸² is hydrogen, or straight-chain or branched C₁₋₆ alkyl, R⁸³ is hydrogen, straight-chain or branched C₁₋₆ alkyl, or phenyl C₁₋₆ alkyl, R^{8a′} is halogen, R⁹, R¹⁰ and X are the same as defined above, can be prepared by reacting a substituted naphthylamine and suitable halogenating agents, for instance, N-halosuccinimides such as N-chlorosuccinimide and N-bromosuccinimide; and N-fluoro-pyridium salts such as N-fluoro-4-methylpyridinium-2-sulfonate, and others.

The reaction may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, and others. Optionally, two or more of the solvents selected from the listed above can be mixed and used.

The reaction temperature can be optionally set depending on the compounds to be reacted. The reaction temperature is usually, but not limited to, about 0° C. to 60° C. The reaction may be conducted for, usually, 30 minutes to 48 hours and preferably 1 to 24 hours. The substituted naphthylamine and halogenating agents are commercially available or can be prepared by the use of known techniques.

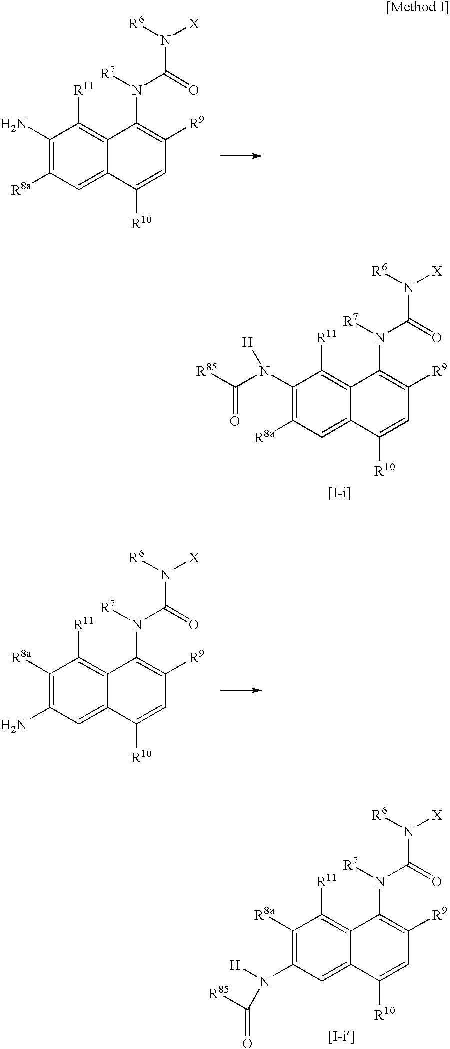

The compound [I-i] and the compound [I-i′], wherein R⁸⁵ represents hydrogen or straight-chain or branched C₁₋₆ alkyl and R⁶, R⁷, R^{8a}, R⁹, R¹⁰, R¹¹ and X is the same as defined above, can be prepared by reacting a substituted naphthylamine and suitable acylating agents, for instance, carboxylic anhydrides such as formic anhydride, and acetic anhydride; acyl halides such as acetyl chloride, and others.

The reaction may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitriles such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction can be advantageously carried out in the presence of a base including, for instance, alkali metal carbonates such as sodium carbonate and potassium carbonate; alkali metal hydrogen carbonates such as sodium hydrogen carbonate and potassium hydrogen carbonate; organic amines such as pyridine, triethylamine and N,N-diisopropylethylamine, and others.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 10 hours.

The substituted naphthylamine and acylating agents are commercially available or can be prepared by the use of known techniques.

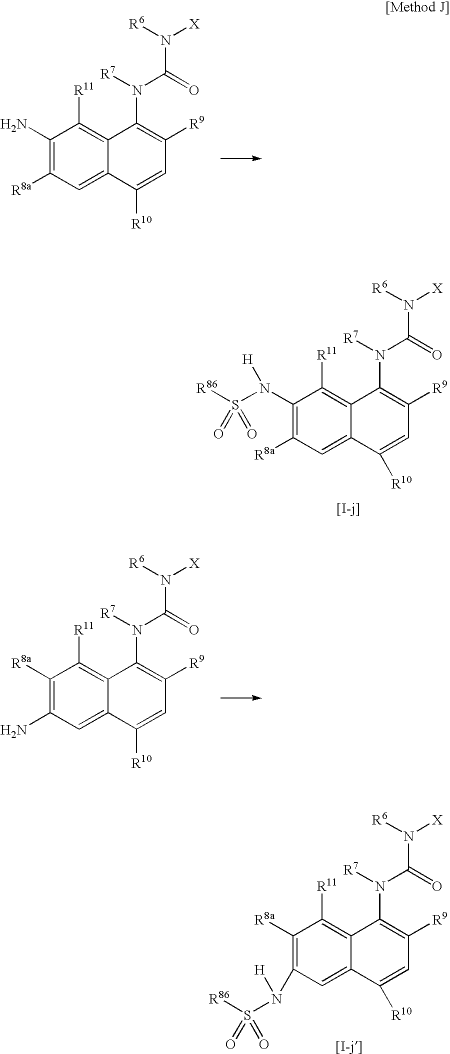

The compound [I-j] and the compound [I-j′], wherein R⁸⁶ is straight-chain or branched C₁₋₆ all and R⁶, R⁷, R^{8a}, R⁹, R¹⁰, R¹¹ and X is the same as defined above, can be prepared by reacting a substituted naphthylamine and alkylsulfonyl chloride such as methanesulfonyl chloride, ethanesulfonyl chloride and others.

The reaction may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction can be advantageously carried out in the presence of a base including, for instance, alkali metal carbonates such as sodium carbonate or potassium carbonate; alkali metal hydrogen carbonates such as sodium hydrogen carbonate and potassium hydrogen carbonate; organic amines such as pyridine, triethylamine and N,N-diisopropylethylamine, and others.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 24 hours.

The substituted naphthylamine and alkylsulfonyl chlorides are commercially available or can be prepared by the use of known techniques.

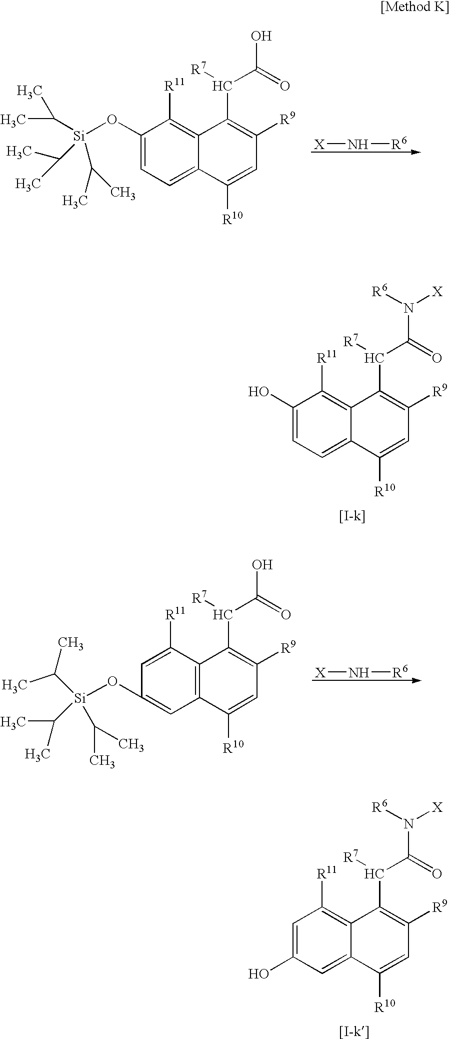

The compound [I-k] and the compound [I-k′], wherein R⁶, R⁷, R⁹, R¹⁰, R¹¹, and X are the same as defined above, can be prepared by (1) the reacting a substituted naphthalene and amine represented by the formula X—NH—R⁶ (wherein R⁶ and X are the same as defined above) (2) adding fluoride salts, such as tetrabutylamonium fluoride to the reaction mixture.

The reaction (1) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction may be carried out using coupling agent including, for instance, carbodiimides such as N,N-dicyclohexylcarbodiimide and 1-(3-dimethylamino-propyl)-3-ethylcarbodiimide, and others.

The reaction may be advantageously carried out in the presence of a base including, for instance, organic amines such as pyridine, 4-dimethlyaminopyridine, triethylamine and N,N-diisopropylethylamine, and others.

The reaction temperature can be optionally set depending on the compounds to be reacted. The reaction temperature is usually, but not limited to, about 0° C. to 60° C. The reaction may be conducted for, usually, 30 minutes to 48 hours and preferably 1 to 24 hours.

The reaction (2) may be carried out in a solvent including, for instance, halogenated hydrocarbons such as dichloromethane, chloroform and 1,2-dichloroethane; ethers such as diethylether, dioxane, tetrahydrofuran (THF) and 1,2-dimethoxyethane; aromatic hydrocarbons such as benzene, toluene and xylene; ketones such as acetone; nitrites such as acetonitrile; amides such as N,N-dimethylformamide (DMF), N,N-dimethylacetamide and N-methylpyrrolidone; sulfoxides such as dimethylsulfoxide (DMSO), and others. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 10 hours and preferably 1 to 24 hours.

The substituted naphthalene, amine, and fluoride salt are commercially available or can be prepared by the use of known techniques.

When the compound shown by the formula (I) or a salt thereof has tautomeric isomers and/or stereoisomers (e.g., geometrical isomers and conformational isomers), each of their separated isomer and mixtures are also included in the scope of the present invention.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, respectively.

Acids to form acid addition salts include inorganic acids such as, without limitation, sulfuric acid, phosphoric acid, hydrochloric acid, hydrobromic acid, hydroiodic acid and the like, and organic acids, such as, without limitation, p-toluenesulfonic acid, methanesulfonic acid, oxalic acid, p-bromophenylsulfonic acid, carbonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassiumcarbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients therefore. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills, powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### BRIEF DESCRIPTION OF DRAWINGS

FIG. 1 presents charts showing bladder capacity and voiding frequency in normal rats, cyclophosphamide treated rats (vehicle) and CYP-VR1 antagonist treated rats.

FIG. 2 presents graphs which shows the bladder capacity in normal rats, cyclophosphamide treated rats (vehicle), and CYP-VR1 antagonist treated rats.

FIG. 3 presents graphs which shows the micturition frequency in normal rats, cyclophosphamide treated rats (vehicle), and CYP-VR1 antagonist treated rats.

### EMBODIMENT OF THE INVENTION

### EXAMPLES

The present invention will be described as a form of examples, but they should by no means be construed as defining the metes and bounds of the present invention.

In the examples below, all quantitative data, if not stated otherwise, relate to percentages by weight.

Mass spectra were obtained using electrospray (ES) ionization techniques (micromass Platform LC). Melting points are uncorrected. Liquid Chromatography—Mass spectroscopy (LC-MS) data were recorded on a Micromass Platform LC with Shimadzu Phenomenex ODS column (4.6 mmφ×30 mm) flushing a mixture of acetonitrile-water (9:1 to 1:9) at 1 ml/min of the flow rate. TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Tokyo kasei kogyo Co., Ltd., Nacalai tesque, Inc., Watanabe Chemical Ind. Ltd., Maybridge plc, Lancaster Synthesis Ltd., Merck KgaA, Kanto Chemical Co.,Ltd.

The effect of the present compounds were examined by the following assays and pharmacological tests.

[Measurement of Capsaicin-induced Ca²⁺ Influx in the Human VR1-transfected CHO Cell Line] (Assay 1)

(1) Establishment of the human VR1-CHOluc9aeq cell line

Human vanilloid receptor (hVR1) cDNA was cloned from libraries of axotomized dorsal root ganglia (WO2000/29577). The cloned hVR1 cDNA was constructed with pcDNA3 vector and transfected into a CHOluc9aeq cell line. The cell line contains aequorin and CRE-luciferase reporter genes as read-out signals. The transfectants were cloned by limiting dilution in selection medium (DMEM/F12 medium (Gibco BRL) supplemented with 10% FCS, 1.4 mM Sodium pyruvate, 20 mM HEPES, 0.15% Sodium bicarbonate, 100 U/ml penicillin, 100 μ/ml streptomycin, 2 mM glutamine, non-essential amino acids and 2 mg/ml G418). Ca²⁺ influx was examined in the capsaicin-stimulated clones. A high responder clone was selected and used for further experiments in the project. The human VR1-CHOluc9aeq cells were maintained in the selection medium and passaged every 3-4 days at 1-2.5×10⁵ cells/flask (75 mm²)

(2) Measurement of Ca²⁺ influx using FDSS-3000

Human VR1-CHOluc9aeq cells were suspended in a culture medium which is the same as the selection medium except for G418 and seeded at a density of 1,000 cells per well into 384-well plates (black walled clear-base/Nalge Nunc International). Following the culture for 48 hrs the medium was changed to 2 μM Fluo-3 AM (Molecular Probes) and 0.02% Puronic F-127 in assay buffer (Hank's balanced salt solution (HBSS), 17 mM HEPES (pH7.4), 1 mM Probenecid, 0.1% BSA) and the cells were incubated for 60 min at 25° C. After washing twice with assay buffer the cells were incubated with a test compound or vehicle for 20 min at 25° C. Mobilization of cytoplasmic Ca²⁺ was measured by FDSS-3000 (λₑₓ=488 nm, λₑₘ=540 nm/Hamamatsu Photonics) for 60 sec after the stimulation with 10 nM of capsaicin (Nacalai Tesque). Integral R of the fluorescence changes was calculated in the samples treated with a test compound and vehicle respectively. Inhibitory effect of the compound was calculated by a comparison of the integral R values.

[Measurement of the Capsaicin-induced Ca²⁺ Influx in Primary Cultured Rat Dorsal Root Ganglia Neurons] (Assay 2)

(1) Preparation of rat dorsal root ganglia neurons

New born Wister rats (5-11 days) were sacrificed and dorsal root ganglia (DRG) was removed. DRG was incubated with 0.1% trypsin (Gibco BRL) in PBS(−) (Gibco BRL) for 30 min at 37° C., then a half volume of fetal calf serum (FCS) was added and the cells were spun down. The DRG neuron cells were resuspended in Ham F12/5% FCS/5% horse serum (Gibco BRL) and dispersed by repeated pipetting and passing through 70 μm mesh (Falcon). The culture plate was incubated for 3 hours at 37° C. to remove contaminating Schwann cells. Non-adherent cells were recovered and further cultured in laminin-coated 384 well plates (Nunc) at 1×10⁴ cells/50 μl/well for 2 days in the presence of 50 ng/ml recombinant rat NGF (Sigma) and 50 μM 5-fluorodeoxyuridine (Sigma).

(2) Ca²⁺ mobilization assay

DRG neuron cells were washed twice with HBSS supplemented with 17 mM HEPES (pH 7.4) and 0.1% BSA. After incubating with 2 μM fluo-3AM (Molecular Probe), 0.02% PF127 (Gibco BRL) and 1 mM probenecid (Sigma) for 40 min at 37° C., cells were washed 3 times. The cells were incubated with VR1 antagonists or vehicle (dimethylsulphoxide) and then with 1 μM of capsaicin (Nacalai Tesque) in FDSS-6000 (λₑₓ=480 nm, λₑₘ=520 nm/Hamamatsu Photonics). The fluorescence changes at 480 nm were monitored for 2.5 min. Integral R of the fluorescence change was calculated in the samples treated with a compound and vehicle, respectively. Inhibitory effect of the compound was calculated by comparison of the integral R-values.

[Organ Bath Assay to Measure the Capsaicin-induced Bladder Contraction] (Assay 3)

Male Wistar rats (10 week old) were anesthetized with ether and sacrificed by dislocating the necks. The whole urinary bladder was excised and placed in oxygenated Modified Krebs-Henseleit solution (pH 7.4) of the following composition (112 mM NaCl, 5.9 mM KCl, 1.2 mM MgCl₂, 1.2 mM NaH₂PO₄, 2 mM CaCl₂, 2.5 mM NaHCO₃, 12 mM glucose). Contractile responses of the urinary bladder were studied as described previously [Maggi C A et al: Br.J.Pharmacol. 108: 801-805, 1993]. Isometric tension was recorded under a load of 1 g using longitudinal strips of rat detrusor muscle. Bladder strips were equilibrated for 60 min before each stimulation. Contractile response to 80 mM KCl was determined at 15 min intervals until reproducible responses were obtained. The response to KCl was used as an internal standard to evaluate the maximal response to capsaicin. The effects of the compounds were investigated by incubating the strips with compounds for 30 min prior to the stimulation with 1 μM of capsaicin (Nacalai Tesque) (vehicle: 80% saline, 10% EtOH, and 10% Tween 80). One of the preparations made from the same animal was served as a control while the others were used for evaluating compounds. Ratio of each capsaicin-induced contraction to the internal standard (i.e. KCl-induced contraction) was calculated and the effects of the test compounds on the capsaicin-induced contraction were evaluated.

[Measurement of Capsaicin-induced Over Active Bladder Contraction in Anesthetized Rats] (Assay 4)

(1) Animals

Female Sprague-Dawley rats (180˜250 g/Charles River Japan) were used.

(2) Catheter implantation

Rats were anesthetized by intraperitoneal administration of urethane (Sigma) at 1.2 g/kg. The abdomen was opened through a midline incision, and a polyethylene catheter (BECTON DICKINSON, PE50) was implanted into the bladder through the dome. In parallel, the inguinal region was incised, and a polyethylene catheter (Hibiki, size 5) filled with 2 IU/ml of heparin (Novo Heparin, Aventis Pharma, France) in saline (Otsuka) was inserted into a femoral vein.

(3) Cystometric investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 3.6 ml/hr. Intravesical pressure was recorded continuously on a chart pen recorder (Yokogawa). At least three reproducible micturition cycles, corresponding to a 20-minute period, were recorded before a test compound administration and used as baseline values.

(4) Administration of test compounds and stimulation of bladder with capsaicin

The saline infusion was stopped before administrating compounds. A testing compound dissolved in the mixture of ethanol, Tween 80 (ICN Biomedicals Inc.) and saline (1:1:8, v/v/v) was administered intraarterially at 3 mg/kg or 10 mg/kg. 2 min after the administration of the compound, saline including 30 μM of capsaicin (Nacalai Tesque) was infused at room temperature into the bladder at a rate of 3.6 ml/hr.

(5) Analysis of cystometry parameters

Relative increases in the capsaicin-induced intravesical pressure were analyzed from the cystometry data. The capsaicin-induced bladder pressures were compared with the maximum bladder pressure during micturition without the capsaicin stimulation. The testing compounds-mediated inhibition of the increased bladder pressures was evaluated using Student's t-test. A probability level less than 5% was accepted as significant difference.

[Measurement of Over Active Bladder in Anesthetized Cystitis Rats] (Assay 5)

(1) Animals

Female Sprague-Dawley rats (180˜250 g/Charles River Japan) were used.

Cyclophosphamide (CYP) dissolved in saline was administered intraperitoneally at 150 mg/kg 48 hours before experiment.

(2) Catheter implantation

Rats were anesthetized by intraperitoneal administration of urethane (Sigma) at 1.25 g/kg. The abdomen was opened through a midline incision, and a polyethylene catheter (BECTON DICKINSON, PE50) was implanted into the bladder through the dome. In parallel, the inguinal region was incised, and a polyethylene catheter (BECTON DICKINSON, PE50) filled with saline (Otsuka) was inserted into a femoral vein. After the bladder was emptied, the rats were left for 1 hour for recovery from the operation.

(3) Cystometric investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 3.6 ml/hr for 20 min. Intravesical pressure was recorded continuously on a chart pen recorder (Yokogawa). At least three reproducible micturition cycles, corresponding to a 20-minute period, were recorded before a test compound administration.

(4) Administration of test compounds

A testing compound dissolved in the mixture of ethanol, Tween 80 (ICN Biomedicals Inc.) and saline (1:1:8, v/v/v) was administered intravenously at 0.05 mg/kg, 0.5 mg/kg or 5 mg/kg. 3 min after the administration of the compound, saline (Nacalai Tesque) was infused at room temperature into the bladder at a rate of 3.6 ml/hr.

(5) Analysis of cystometry parameters

The cystometry parameters were analyzed as described previously [Lecci A et al: Eur. J. Pharmacol. 259: 129-135, 1994]. The micturition frequency calculated from micturition interval and the bladder capacity calculated from a volume of infused saline until the first micturition were analyzed from the cystometry data The testing compounds-mediated inhibition of the frequency and the testing compounds-mediated increase of bladder capacity were evaluated using unpaired Student's t-test. A probability levels less than 5% was accepted as significant difference. Data were analyzed as the mean±SEM from 4-7 rats.

Selectivity Test

[Measurement of Ca²⁺ Influx in the Human P2X1-transfected CHO Cell Line]

(1) Preparation of the human P2X1-transfected CHOluc9aeq cell line

Human P2X1-transfected CHOluc9aeq cell line was established and maintained in Dulbecco's modified Eagle's medium (DMEM/F12) supplemented with 7.5% FCS, 20 mM HEPES-KOH (pH 7.4), 1.4mM sodium pyruvate, 100 U/ml penicillin, 100 μg/ml streptomycin, 2 mM glutamine (Gibco BRL) and 0.5 Units/ml apyrase (grade I, Sigma). The suspended cells were seeded in each well of 384-well optical bottom black plates (Nalge Nunc International) at 3×10³/50 μl/well. The cells were cultured for following 48 hrs to adhere to the plates.

(2) Measurement of the intracellular Ca²⁺ levels n

P2X1 receptor agonist-mediated increases in cytosolic Ca²⁺ levels were measured using a fluorescent Ca²⁺ chelating dye, Fluo-3 AM (Molecular Probes). The plate-attached cells were washed twice with washing buffer (HBSS, 17 mM HEPES-KOH (pH 7.4), 0.1% BSA and 0.5 units/ml apyrase), and incubated in 40 μl of loading buffer (1 μM Fluo-3 AM, 1 mM probenecid, 1 μM cyclosporin A, 0.01% pluronic (Molecular Probes)in washing buffer) for 1 hour in a dark place. The plates were washed twice with 40 μl washing buffer and 35 μl of washing buffer were added in each well with 5 μl of test compounds or 2′,3′-o-(2,4,6-trinitrophenyl) adenosine 5′-triphosphate (Molecular Probes) as a reference. After further incubation for 10 minutes in dark 200 nM α,β-methylene ATP agonist was added to initiate the Ca²⁺ mobilization. Fluorescence intensity was measured by FDSS-6000 (λₑₓ=100 nm, λₑₘ=510 nm /Hamamatsu Photonics) at 250 msec intervals. Integral ratios were calculated from the data and compared with that of a control.

All of the compounds in the examples were examined in the assays. The data corresponds to the compounds as yielded by solid phase synthesis and thus to levels of purity of about 40 to 90%. Almost all of the compounds (more than 95% of the compounds) disclosed in the Examples below and tables below show IC₅₀ value of equal or below 1 μM. Among others, the following compounds:

N-(7-hydroxy-1-naphthyl)-N′-[4-(trifluoromethyl)phenyl]urea;

N-(7-hydroxy-1-naphthyl)-N′-(4-phenoxyphenyl)urea;

N-[4-chloro-3-(trifluoromethyl)phenyl]-N′-(7-hydroxy-1-naphthyl)urea;

N-[4-(4-chlorophenoxy)phenyl]-N′-(7-hydroxy-1-naphthyl)urea;

N-(1,1′-biphenyl-3-yl)-N′-(7-hydroxy-1-naphthyl)urea;

N-(7-hydroxy-1-naphthyl)-N′-(3-phenoxyphenyl)urea;

N-(3-chlorophenyl)-N′-(2,4-dibromo-7-hydroxy-1-naphthyl)urea;

N-[4-chloro-3-(trifluoromethyl)phenyl]-N′-(2,4-dibromo-7-hydroxy-1-naphthyl)urea;

N-(4-bromobenzyl)-N′-(2-chloro-7-hydroxy-1-naphthyl)urea;

N-(2-chloro-7-hydroxy-1-naphthyl)-N′-[4-chloro-3-(trifluoromethyl)phenyl]urea;

N-[4-chloro-3-(trifluoromethyl)phenyl]-N′-(2,4-dichloro-7-hydroxy-1-naphthyl)urea;

N-(1,1′-biphenyl-3-yl)-N′-(2-chloro-7-hydroxy-1-naphthyl)urea;

ethyl 3-({[(2,4-dichloro-7-hydroxy-1-naphthyl)amino]carbonyl}amino)benzoate;

N-(2,4-dichloro-7-hydroxy-1-naphthyl)-N′-(2-naphthyl)urea;

N-(2,4-dichloro-7-hydroxy-1-naphthyl)-N′-[3-(trifluoromethyl)phenyl]urea;

N-(2′-chloro-1,1′-biphenyl-3-yl)-N′-(2,4-dichloro-7-hydroxy-1-naphthyl)urea;

N-(4bromo-2-chloro-7-hydroxy-1-naphthyl)N′-[4-chloro-3-(trifluoromethyl)phenyl]urea;

N-(2,4-dichloro-7-hydroxy-1-naphthyl)-N′-[4-fluoro-3-(trifluoromethyl)phenyl]urea;

N-[4-chloro-3-(trifluoromethyl)phenyl]-N′-(7-hydroxy-4-methyl-1-naphthyl)urea; and

N-(2-chloro-7-hydroxy-4-methyl-1-naphthyl)-N′-[4-chloro-3-(trifluoromethyl)phenyl]urea

or the salt thereof (e.g., potassium salt) show IC₅₀ value of equal to or below 10 nM.

The compounds of the present invention also show excellent selectivity, and strong activity in other assays (2)-(4) described above.

Preparing Method of Starting Compounds

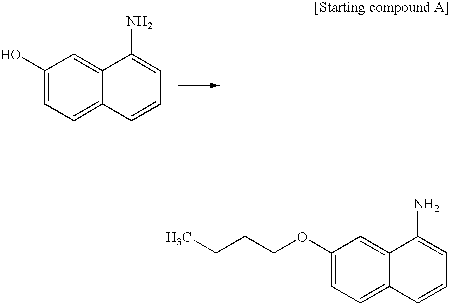

To a stirred solution of 8-amino-2-naphthol (0.050 g, 0.314 mmol), tetrabutyl-ammonium iodide (0.012 g, 0.031 mmol) and 1-bromobutane (0.04 mL, 0.346 mmol) in acetone (2 mL) was added potassium carbonate (0.130 g, 0.942 mmol). The mixture was stirred at room temperature for one day, then warm to 60° C. for one day and diluted with AcOEt. The mixture was extracted with ethyl acetate and water. Then the layers are separated. The separated organic phase was washed with brine, dried over Na₂SO₄, filtered, and concentrated under reduced pressure. The resulting residue was purified by preparative thin layer chromatography on silica gel (hexane/ethyl acetate=4/1) to give 7-butoxy-1-naphthylamine (0.040 g, 59%).

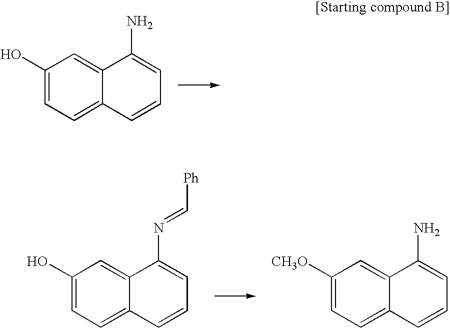

A mixture of 8-amino-2-naphthol (1.0 g, 6.28 mmol), benzaldehyde (0.73 g, 6.91 mmol) and Na₂SO₄ (5.0 g, 35.20 mmol) in boiling THF (12 ml) was stirred overnight. The mixture was filtered and concentrated under reduced pressure. The resulting residue was purified by flash chromatography on silica gel (Hex/AcOEt/Et₃N=75/23/2) to give 8-{[(1E)-phenylmethylidene]amino}-2-naphthol (1.52 g, yield 98%) as a yellow solid.

Next, A mixture of 8-{[(1E)-phenylmethylidene]amino}-2-naphthol (0.50 g, 2.02 mmol), MeI (0.57 g, 4.04 mmol), and NaOH (0.24 g, 6.06 mmol) in acetone was stirred at room temperature for 2 hrs. The resulting mixture was concentrated, and the residue was dissolved in Et₂O, washed with water and brine and then concentrated under reduced pressure. The residue was dissolved in 2N HCl-THF (30 ml, 2:1) and stirred at room temperature for 1.5 hrs. The resulting solution was washed with Et₂O. The aqueous layer was basified with Na₂CO₃, extracted with Et₂O. The organic layer was washed with brine, dried over Na₂SO₄, filtered and concentrated under reduced pressure. The resulting residue was purified by flash chromatography on silica gel (Hex/AcOEt=3/1) to give 7-methoxy-1-naphthylamine (0.33 g 93%) as a white solid.

With the use of EtI, iPrBr, or Bromomethyl-cyclopropane instead of MeI, 7-ethoxy-1-naphthylamine, 7-propyl-1-naphthylamine, or 7-(cyclopropylmethoxy)-1-naphthylamine, was prepared, respectively.

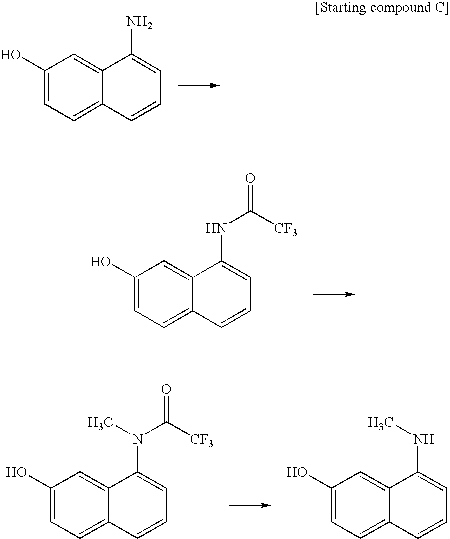

To a solution of 8-amino-2-naphthol (10.62 g, 62.82 mmol) and pyridine (9.94 g, 125.64 mmol) in dry dioxane (300 ml) was added at 0° C. trifluoroacetic anhydride (19.79g, 94.23 mmol). The solution was allowed to warm to room temperature and stirred for 1.5 hrs. The resulting solution was concentrated. The residue was dissolved in Et₂O, washed with 1N HCl and brine, dried with Na₂SO₄, filtered, and concentrated under reduced pressure. The resulting residue was purified by flash chromatography on silica gel (hexane: AcOEt=6:1) to give 2,2,2-trifluoro-N-(7-hydroxy-1-naphthyl)acetamide (4.73g, 30%) as a purple solid.

Next, A mixture of 2,2,2-trifluoro-N-(7-hydroxy-1-naphthyl)acetamide (0.50 g, 1.96 mmol), MeI (0.31 g, 2.16 mmol), K₂CO₃ (1.35 g, 9.80 mmol) and TBAI (0.072 g, 0.196 mmol) in acetone (10 ml) was stirred at room temperature for 2.5 hrs. The resulting mixture was filtered and concentrated. The residue was diluted with AcOEt and washed with brine, dried with Na₂SO₄, filtered, and concentrated. The resulting residue was purified by flash chromatography on silica gal (hexane/AcOEt=10/1 then 4/1) to give 2,2,2-trifluoro-N-(7-hydroxy-1-naphthyl)-N-methylacet-amide (0.33 g, 63%) as a white solid.

Next, To a solution of 2,2,2-trifluoro-N-(7-hydroxy-1-naphthyl)-N-methylacetamide (0.058 g, 0.22 mmol) in EtOH (3 ml) was added NaBH₄ (0.15 g, 0.215 mmol). The reaction mixture was stirred at room temperature until TLC showed no starting material present. The solution was concentrated. The residue was dissolved in Et₂O, washed with H₂O and brine, dried with Na₂SO₄, filtered, and concentrated under reduced pressure. The resulting residue was purified by flash chromatography on silica gel (hexane/AcOEt=4/1) to give 8-(methylamino)-2-naphthol (0.032 g, 87%) as a white solid.

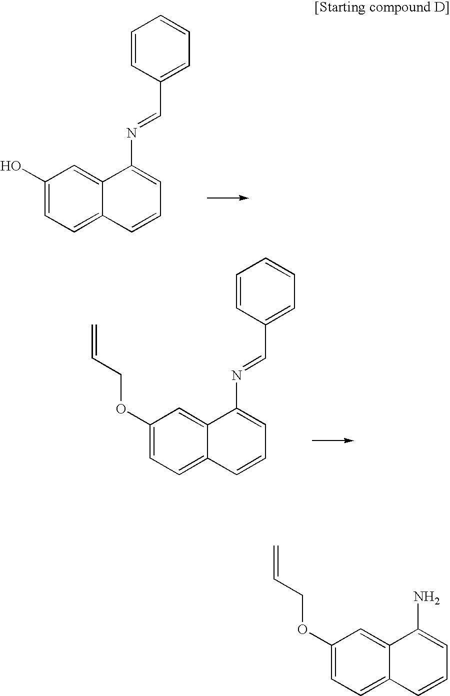

To a suspension of 8-{[(1E)-phenylmethylidene]amino}-2-naphthol, which was prepared in the step (1) of the process of preparing the starting compound B, (236 mg, 0.95 mmol) and K₂CO₃ (263 mg, 1.90 mmol) in 10 mL of DMF was added allylbromide (150 mg, 1.24 mmol) at room temperature. After 3 hrs, the reaction mixture was poured into water (50 mL) and extracted with Et₂O. The combined organic layers were washed with water, brine, dried over MgSO₄, and concentrated under reduced pressure. The residue was purified by column chromatography (hexane/EtOAc=1/10) to give 7-(allyloxy)-N-[(1E)-phenylmethylidene]-1-naphthalenamine (259 mg, 95%) as a solid.

Next, obtained 7-(allyloxy)-N-[(1E)-phenylmethylidene]-1-naphthalenamine was dissolved in the mixture of THF and aqueous 2N HCl solution (20 mL, 1:3). After 1 hr stirring at room temperature, the solvent was removed under reduced pressure and the aqueous phase was extracted with Et₂O, and the organic layers was discarded. The aqueous phase was alkalized with aqueous 1N NaOH solution, and then the mixture was extracted with EtOAc. The EtOAc solution was dried over Na₂SO₄ and then concentrated under reduced pressure to give the crude product. Then the crude product was purified by column chromatography on silica gel(hexane/EtOAc=1/8 then 1/5) to give 7-(allyloxy)-1-naphthylamine (128.5 mg, 66%) as a solid.

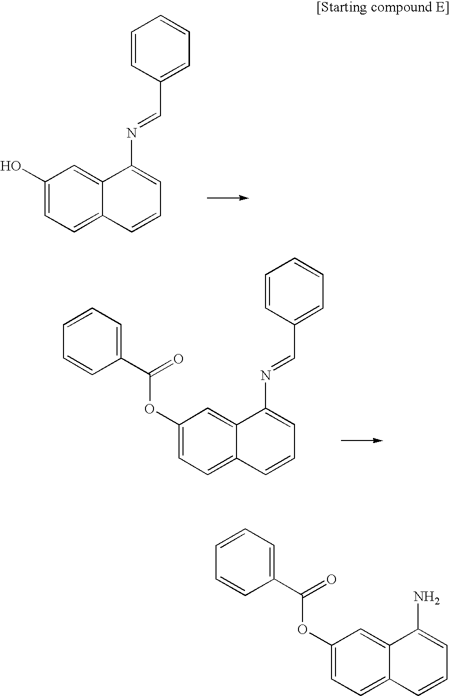

To a mixture of 8-{[(1E)-phenylmethylidene]amino}-2-naphthol, which was prepared in the step (1) of the process of preparing starting compound B, (101 mg, 0.45 mmol), benzoyl chloride (70 mg, 0.50 mmol) in 20 mL of CH₂Cl₂ was added TEA (68 mg, 0.65 mmol) at 0° C. The reaction mixture was stirred at room temperature for 1 hr. After removal of the solvent, the residue was washed with hexane.

The obtained crude product was dissolved in a mixture of THF (5 mL) and aqueous 2N HCl solution (10 mL). After 1 hr of stirring at room temperature, the solvent was removed in vacuo and the aqueous phase was extracted Et₂O, and the organic layer was discarded. The aqueous phase was alkalized with aqueous 1N NaOH solution and then the mixture was extracted with EtOAc. The EtOAc solution was dried over Na₂SO₄ and then concentrated under reduced pressure to give the crude product. Then the crude product was recrystallized from Et₂O to give 8-amino-2-naphthyl benzoate (108 mg, 92%) as a solid.

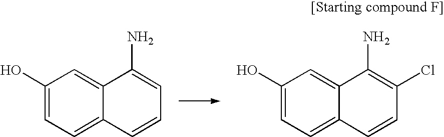

To a stirred solution of 8-amino-2-naphthol (5.00 g, 31.4 mmol) in tetrahydrofuran (100 mL) was added n-chlorosuccinimide (4.19 g, 31.4 mmol). The mixture was stirred at room temperature for 16 hours. Water was added to the mixture, and the product was extracted with ethylacetate. The organic layer was washed with water and brine, dried over Na₂SO₄, filtered, and concentrated under reduced pressure to afford 8-amino-7-chloro-2-naphthol (4.2 g, 69% yield).

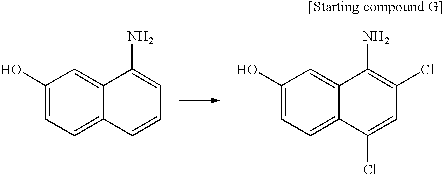

To a stirred solution of 8-amino-2-naphthol(2.00 g, 12.6 mmol) in tetrahydrofuran (50 mL) was added N-chlorosuccinimide (3.69 g, 27.6 mmol). The mixture was stirred at room temperature for 16 hours. Water was added to the mixture, and the product was extracted with ethylacetate. The organic layer was washed with water and brine, dried over Na₂SO₄, filtered, and concentrated under reduced pressure to afford 8-amino-5,7-dichloro-2-naphthol (2.0 g, 70% yield).

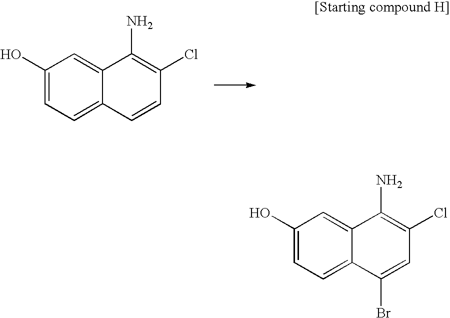

To a stirred solution of 8-amino-7-chloro-2-naphthol (500 mg, 2.58 mmol) in tetrahydrofuran (8 mL) was added N-bromosuccinimide (460 mg, 2.58 mmol). The mixture was stirred at room temperature for 16 hours. Water was added to the mixture, and the product was extracted with ethylacetate. The organic layer was washed with water and brine, dried over Na₂SO₄, filtered, and concentrated under reduced pressure to afford 8-amino-5-bromo-7-chloro-2-naphthol (289 mg, 41% yield).

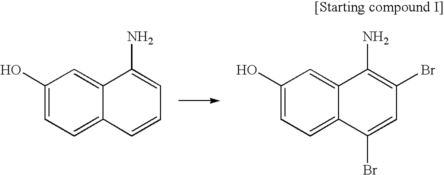

To a stirred solution of 8-amino-2-naphthol (10.0 g, 62.8 mmol) in tetrahydrofuran (300 mL) was added N-bromosuccinimide (22.4 g, 126 mmol) at 0° C. The mixture was stirred at room temperature for 16 hours. Water was added to the mixture, and the product was extracted with ethylacetate. The organic layer was washed with water and brine, dried over Na₂SO₄, filtered, and concentrated under reduced pressure to afford 8-amino-5,7-dibromo-2-naphthol (5.1 g, 26% yield).

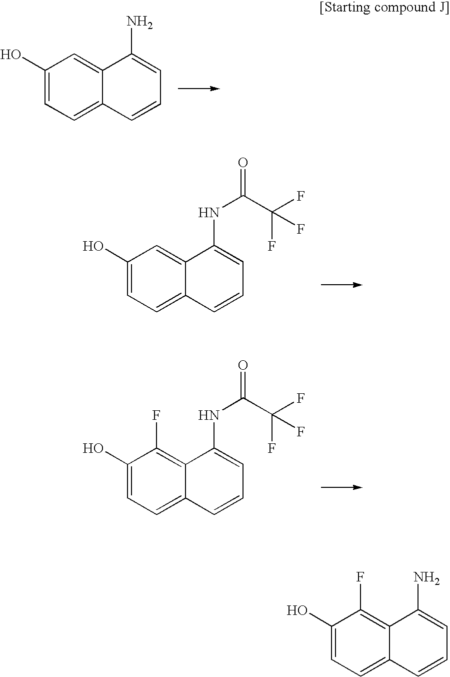

To a solution of 8-amino-2-naphthol (1.59 g, 9.99 mmol) and pyridine (2 mL) in 1,4-dioxane (10 mL) was added trifluoroacetic anhydride (3.15 g, 15.0 mmol) in 1,4-dioxane (5 mL) at 0° C. After stirred for 16 hours, methanol (5 mL) was added and stirred for 5 minutes. An aqueous solution of 1N HCl was added to the mixture and the product was extracted with ethylacetate. The organic layer was concentrated under reduced pressure, and the residue was purified by silica gel column chromatography (hexane:ethylacetate, 3:1) to give 2,2,2-trifluoro-N-(7-hydroxy-1-naphthyl)acetamide (2.19 g, 86% yield).

Next, a mixture of 2,2,2-trifluoro-N-(7-hydroxy-1-naphthyl)acetamide (500 mg, 1.96 mmol) and N-fluoro-6-(trifluoromethyl)pyridinium-2-sulfonate (504 mg, 2.06 mmol) in 1,1,2-trichloroethane (5 mL) was stirred at 50° C. for 18 hours. The mixture was poured into water. The product was extracted with diethylether, and the organic layer was washed with brine, dried with MgSO₄, filtered, and concentrated under reduced pressure. The residue was purified by silica gel column chromatograpy (chloroform: methanol, 50:1) to give 2,2,2-trifluoro-N-(8-fluoro-7-hydroxy-1-naphthyl)acetamide (200 mg, 37% yield).

Next, a solution of 2,2,2-trifluoro-N-(8-fluoro-7-hydroxy-1-naphthyl)acetamide (194 mg, 0.710 mmol) in saturated ammonia in methanol was stirred at room temperature for 18 hours. The mixture was concentrated under reduced pressure, and the residue was purified by column chromatography (hexane:ethylacetate, 2:1) to give 8-amino-1-fluoro-2-naphthol (119 mg, 95% yield).

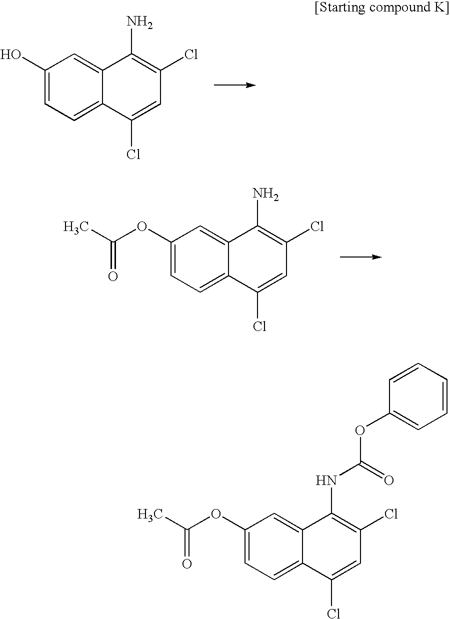

To a solution of 8-amino-5,7-dichloro-2-naphthol (2.28 g, 10.0 mmol) and pyridine (0.949 g, 12 mmol) in dichloromethane (30 mL) was added dropwised a solution of acetic anhydride (1.07 g, 10.5 mmol) at 0° C. The mixture was stirred for 5 hours at room temperature. To the mixture was added water, and then extracted with dichloromethane. The organic layer was dried with Na₂SO₄, and concentrated in vacuo. The residue was washed with n-hexane to give 8-amino-5,7-dichloro-2-naphthyl acetate (2.4 g, 89%).

Next, to the solution of 8-amino-5,7-dichloro-2-naphthyl acetate (2.41 g, 8.93 mmol) and pyridine (0.847 g, 10.7 mmol) in THF (27 mL) was added phenyl chloroformate (1.47 g, 9.38 mmol) at room temperature. The mixture was stirred for 2.5 hours at 50° C. To the reaction mixture was added ethylacetate and washed with water and brine. The organic layer was concentrated in vacuo. The residue was washed with n-hexane to give 5,7-dichloro-8-[(phenoxycarbonyl)amino]-2-naphthyl acetate (3.19 g, 92%).

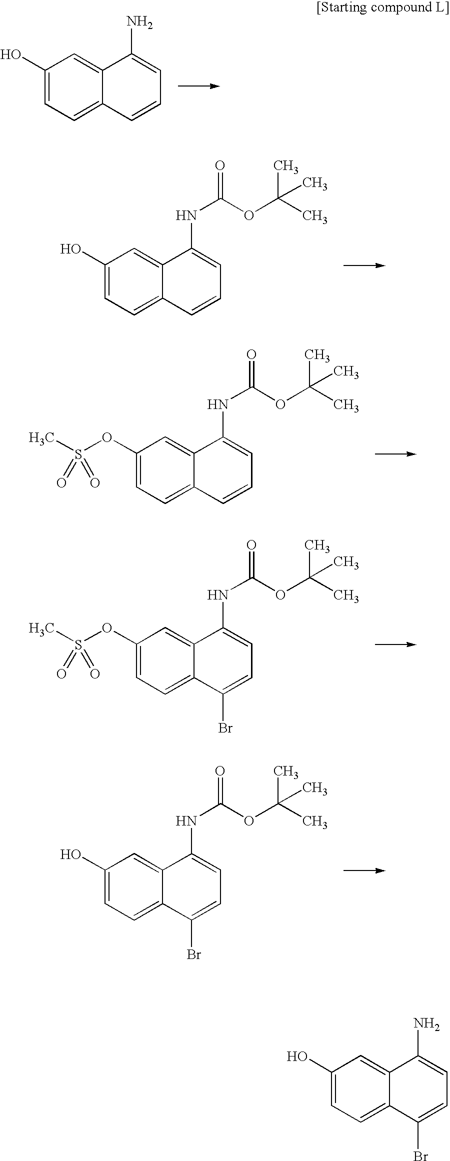

To a stirred solution of 8-amino-2-naphthol (5.00 g, 31.4 mmol) in a mixture of tetrahydrofuran (50 mL) and dichloromethane (100 mL) was added di-t-butyl-dicarbonate (6.86 g, 31.4 mmol). The mixture was stirred at 70° C. for 18 hours. After the mixture was cooled to room temperature, saturated aqueous solution of sodium carbonate was added and the product was extracted with dichloromethane. The organic layer was washed with water and brine, dried over Na₂SO₄, filtered, and concentrated under reduced pressure. The obtained residue was purified by silica gel column chromatography (dichloromethane:ethylacetate, 9:1) to afford tert-butyl 7-hydroxy-1-naphthylcarbamate (5.4 g, 66% yield).

Next, to a mixture of tert-butyl 7-hydroxy-1-naphthylcarbamate (4.67 g, 18.0 mmol) and triethylamine (2.77 g, 27.4 mmol) in dichloromethane (170 mL) was added methanesulfonic anhydride (3.56 g, 19.8 mmol) at 0° C. The mixture was stirred for 30 minutes and poured into saturated aqueous sodium bicarbonate solution. The organic layer was extracted, dried over Na₂SO₄, filtered and concentrated under reduced pressure to give 8-[(tert-butoxycarbonyl)amino]-2-naphthyl methanesulfonate (5.8 g, 95% yield).

Next, to a solution of 8-[(tert-butoxycarbonyl)amino]-2-naphthyl methanesulfonate (2.05 g, 6.08 mmol) in 50 mL acetic acid was added N-bromosuccinimide (1.14 g, 6.41 mmol). The mixture was stirred for 2 hours, and water (100 mL) and dichloromethane (100 mL) were added. The aqueous layer was adjusted to pH 7 by addition of 10 N aqueous sodium hydroxide. The organic layer was extracted, dried over Na₂SO₄, filtered, and concentrated under reduced pressure. The residue was triturated with a mixture of hexane and ethylacetate to give 5-bromo-8-[(tert-butoxycarbonyl)amino]-2-naphthyl methanesulfonate (1.8 g, 71% yield).

Next, a mixture of 5-bromo-8-[(tert-butoxycarbonyl)amino]-2-naphthyl methanesulfonate (1.77 g, 4.24 mmol) and 10% aqueous sodium hydroxide solution (85 mL) in tetrahydrofuran (50 mL) was stirred at 50° C. for 60 hours. The mixture was cooled to 0° C. and neutralized with concentrated hydrochloric acid. The mixture was concentrated under reduced pressure, and the product was extracted with ethylacetate. The organic layer was passed through Celite, dried over Na₂SO₄, filtered, and concentrated under reduced pressure to give tert-butyl 4-bromo-7-hydroxy-1-naphthylcarbamate (1.3 g, 90% yield).

Next, a mixture of tert-butyl 4-bromo-7-hydroxy-1-naphthylcarbamate (198 mg, 0.585 mmol) in 4 N HCl in 1,4-dioxane (5 mL) was stirred for 1 hour. The mixture was concentrated under reduced pressure and was added ethylacetate and saturated aqueous sodium bicarbonate solution. The extracted organic layer was washed with water and brine, dried over Na₂SO₄, filtered, and concentrated under reduced pressure to give 8-amino-5-bromo-2-naphthol (143 mg, 100% yield).

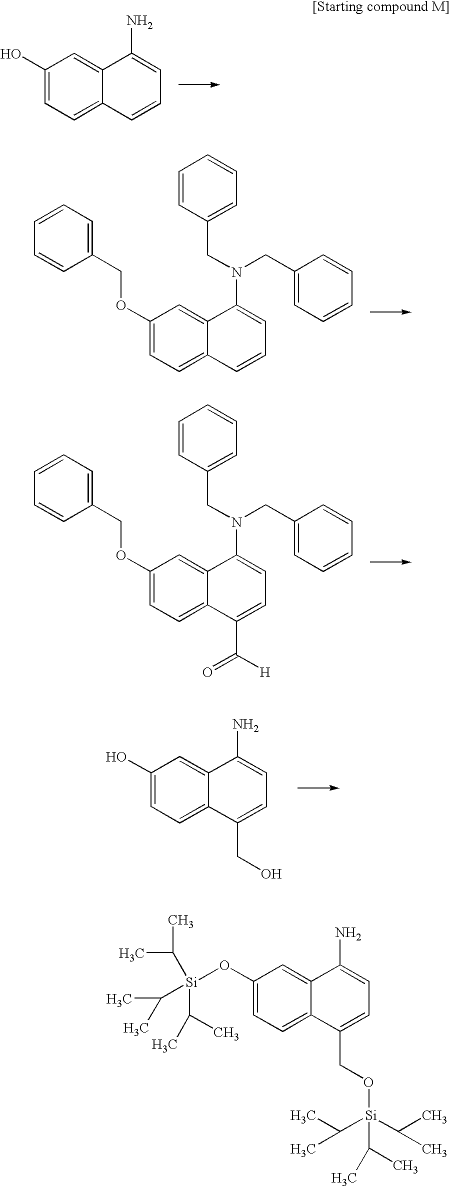

To a stirred mixture of 8-amino-2-naphthol (24.2 g, 152.0 mmol) and Potassium carbonate in acetone (350 mL) was added benzyl bromide (117.0 g, 684.1 mmol) at 0° C. The mixture was refluxed for 48 hours. After the mixture was cooled to room temperature, the mixture was filtered and the filtrate was concentrated in vacuo. To the resulted residue was added diethyl ether, and the precipitates were collected and dried to afford N,N-dibenzyl-7-(benzyloxy)-1-naphthalenamine (50.9 g, 78% yield).

Next, to a stirred solution of N,N-dimethylformamide (100 mL) was added Phosphorus oxychloride (61.2 g, 399.2 mmol) over 30 minutes at 0° C. After stirred for 30 minutes, to the mixture was added N,N-dibenzyl-7-(benzyloxy)-1-naphthalenamine (49.0 g, 114.1 mmol) in N,N-dimethylformamide (400 mL). The mixture was stirred at room temperature for 16 hours, and then poured into ice-water. The product mixture was extracted with dichloromethane, and the organic layer was washed with water, aqueous sodium bicarbonate, and brine. After dried over Na₂SO₄, filtered, and concentrated under reduced pressure, the residue was mixed with ethylacetate and hexane. The precipitates were collected and dried to give 6-(benzyloxy)-4-(dibenzylamino)-1-naphthaldehyde (45.1 g, 86% yield).

Next, to a mixture of 6-(benzyloxy)-4-(dibenzylamino)-1-naphthaldehyde (3.00 g, 6.56 mmol) and 10% Pd/Carbon (0.10 g) in methanol (30 mL) was stirred under hydrogen for 3 days. The mixture was passed through Celite, and the filtrate was concentrated under reduced pressure. The obtained residue was purified by column chromatography (silica gel, 1:1 hexane/ethylacetate) to give 8-amino-5-(hydroxymethyl)-2-naphthol (0.95 g, 76% yield).

Next, to a mixture of 8-amino-5-(hydroxymethyl)-2-naphthol (0.95 g, 5.02 mmol), imidazole (0.75 g, 11.1 mmol), and 4-dimethlyaminopyridine (0.06 g, 0.50 mmol) in N,N-dimethylformamide (10 mL) was added chlorotriisopropylsilane (2.03 g, 10.5 mmol) at 0° C. After the mixture was stirred at room temperature for 16 hours, water was added, and the product was extracted with diethylether. The organic layer was washed with aqueous 10% citric acid, saturated aqueous sodium bicarbonate, and then with brine. The solvent was removed under reduced pressure, and the obtained residue was purified by column chromatography (silica gel, 10:1 hexane/ethylacetate) to give 7-[(triisopropylsilyl)oxy]-4-{[(triisopropylsilyl)oxy]methyl})1-naphthylamine (1.67 g, 66% yield).

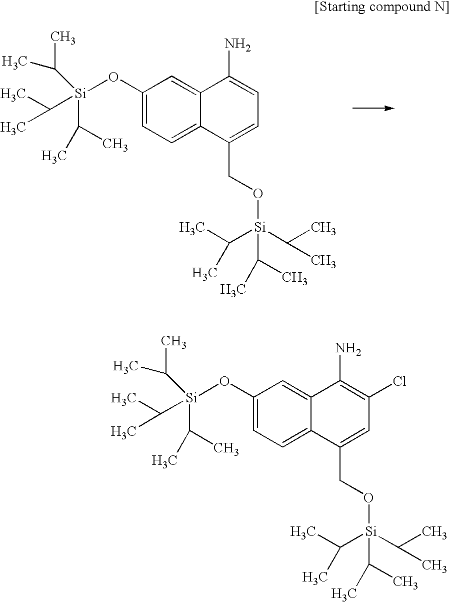

To a stirred solution of 7-[(triisopropylsilyl)oxy]-4-{[(triisopropylsilyl)oxy]methyl}-1-naphthylamine (300 mg, 0.60 mmol) in tetrahydrofuran (3.0 mL) was added N-chlorosuccimide (95.8 mg, 0.72 mmol) at 0° C. The mixture was stirred for 2 hours, and then saturated aqueous sodium bicarbonate was added. The mixture was extracted with ethylacetate, and the organic layer was washed with brine, dried over Na₂SO₄, filtered, and concentrated under reduced pressure. The obtained residue was purified by column chromatography (silica gel, 19:1 hexane/ethylacetate) to give 2-chloro-7-[(triisopropylsilyl)oxy]-4-{[(triisopropylsilyl)oxy]methyl}-1-naphthylamine (112 mg, 35% yield).

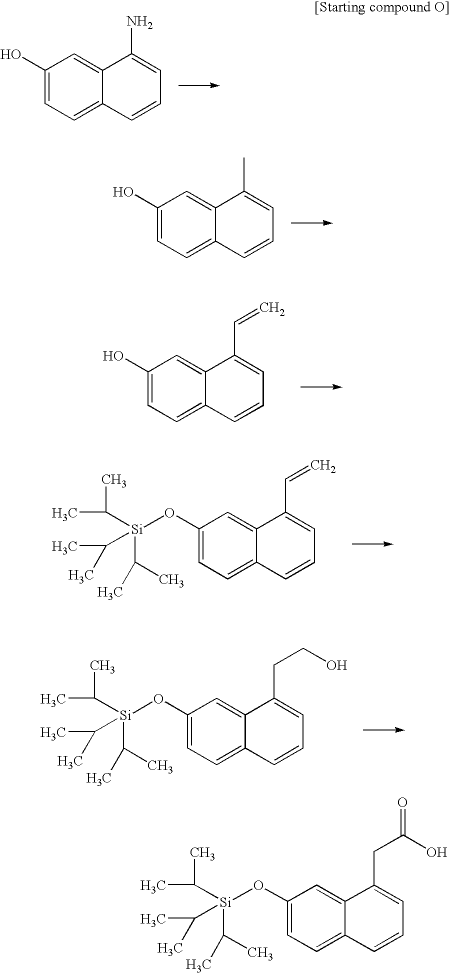

To a mixture of 8-amino-2-naphthol (10.0 g, 62.8 mmol) in tetrahydrofuran (50 mL) and aqueous 3 N hydrochloric acid (100 mL) was added sodium nitrite (4.77 g, 69.1 mmol) in water (15 mL) at 0° C. After stirred for 15 minutes, a solution of potassium iodide (20.8 g, 125.6 mmol) in water (15 ML) was added, and the mixture was stirred at 0° C. for 1 hour. To the reaction mixture was added ethylacetate, and filtered. The filtrate was washed with water, and the organic layer was dried over MgSO₄, filtered, and concentrated under reduced pressure. The obtained residue was purified by silica gel column chromatography (hexane: ethylacetate, 4:1) to give 8-iodo-2-naphthol (4.41 g, 26% yield).

Next, a mixture of 8-iodo-2-naphthol (2.00 g, 7.41 mmol), tributyl(vinyl)tin (2.82 g, 8.89 mmol), and tetrakis(triphenylphosphine)palladium(0) (0.171 g, 0.148 mmol) in toluene (15 mL) was stirred at 90° C. for 16 hours. The mixture was poured into water and extracted with ethylacetate. The organic layer was dried over Na₂SO₄, filtered, and concentrated under reduced pressure. The obtained residue was purified by silica gel column chromatography (hexane:ethylacetate, 10:1) to give 8-vinyl-2-naphthol (1.26 g, 100% yield).

Next, to a solution of 8-vinyl-2-naphthol (1.38 g, 8.10 mmol) and imidazole (0.827 g, 12.1 mmol) in N,N-dimethylformamide (10 mL) was added chlorotriisopropylsilane (1.87 g, 9.72 mmol) at room temperature. The mixture was stirred at 50° C. for 16 hours and was poured into water and extracted with ethylacetate. The organic layer dried over Na₂SO₄, filtered, and concentrated under reduced pressure. The obtained residue was purified by silica gel column chromatography (hexane) to give triisopropyl-[(8-vinyl-2-naphthyl)oxy]silane (1.65 g, 63% yield).

Next, to a solution of triisopropyl-[(8-vinyl-2-naphthyl)oxy]silane (0.500 g, 1.53 mmol) in tetrahydrofuran (3 mL) was added 0.5 M 9-borabicyclo[3.3.1]nonane in tetrahydrofuran (3.0 mL) at 0° C. The mixture was stirred at room temperature for 5 hours, then 3 N aqueous sodium hydroxide (3.0 mL) and 35% aqueous hydrogen peroxide (3.0 mL) were added, and stirred at room temperature for 16 hours. To the mixture was added ethylacetate, and the extracted organic layer was washed with brine, dried over MgSO₄, filtered, and concentrated under reduced pressure. The obtained residue was purified by silica gel column chromatography (hexane:ethylacetate, 10:1) to give 2-{7-[(triisopropylsilyl)oxy]-1-naphthyl}ethanol (0.296 g, 56% yield).

Next, a stock solution of periodic acid (11.4 g, 50.0 mmol) and chromium(VI)oxide (23.0 mg) in 114 mL of acetonitrile (0.75 volume % water) was prepared. To a solution of 2-{7-[(triisopropylsilyl)oxy]-1-naphthyl}ethanol (59.0 mg, 0.171 mmol) in acetonitrile (1 mL) was added the periodic acid/chromium(VI)oxide stock solution (1.0 mL) at 0° C. After stirred for 30 minutes, aqueous solution of sodium hydrogenphosphate (60.0 mg, in 1.0 mL water) and toluene (1.5 mL) were added. The organic layer was separated and washed with brine and aqueous sodium hydrogensulfate, dried over MgSO₄, filtered, and concentrated under reduced pressure. The obtained residue was purified by silica gel column chromatography (hexane:ethylacetate, 4:1) to give {7-[(triisopropylsilyl)oxy]-1-naphthyl}acetic acid (15.0 mg, 24% yield).

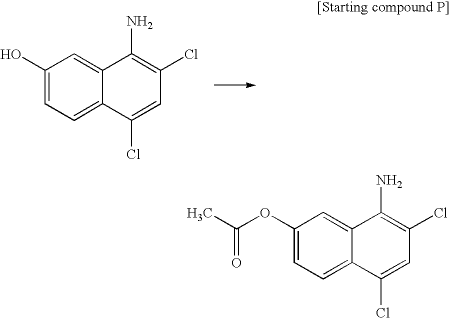

To a solution of 8-amino-5,7-dichloro-2-naphthol (2.28 g, 10.0 mmol) and pyridine (0.949 g, 12 mmol) in dichloromethane (30 mL) was added dropwised a solution of acetic anhydride (1.07 g, 10.5 mmol) at 0 ° C. The mixture was stirred for 5 hours at room temperature. To the mixture was added water, and then extracted with dichloromethane. The organic layer was dried with Na₂SO₄, and concentrated in vacuo. The residue was washed with n-hexane to give 8-amino-5,7-dichloro-2-naphthyl acetate (2.4 g, 89%).

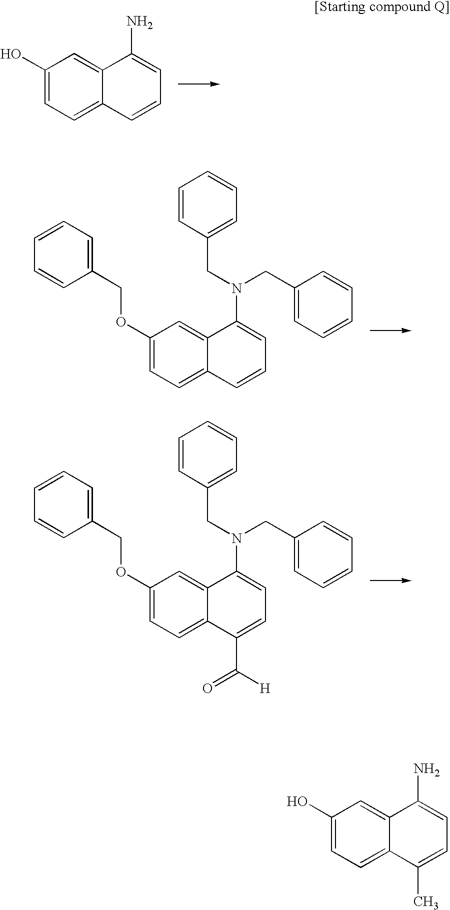

To a stirred mixture of 8-amino-2-naphthol (24.2 g, 152.0 mmol) and Potassium carbonate in acetone (350 mL) was added benzyl bromide (117.0 g, 684.1 mmol) at 0° C. The mixture was refluxed for 48 hours. After the mixture was cooled to room temperature, the mixture was filtered and the filtrate was concentrated in vacuo. To the resulted residue was added diethyl ether, and the precipitates were collected and dried to afford N,N-dibenzyl-7-(benzyloxy)-1-naphtalenlamine (50.9 g, 78% yield).

Next, to a stirred solution of N,N-dimethylformamide (100 mL) was added Phosphorus oxychloride (61.2 g, 399.2 mmol) over 30 minutes at 0° C. After stirred for 30 minutes, to the mixture was added N,N-dibenzyl-7-(benzyloxy)-1-naphthalenamine (49.0 g, 114.1 mmol) in N,N-dimethylformamide (400 mL). The mixture was stirred at room temperature for 16 hours, and then poured into ice-water. The product mixture was extracted with dichloromethane, and the organic layer was washed with water, aqueous sodium bicarbonate, and brine. After dried over Na₂SO₄, filtered, and concentrated under reduced pressure, the residue was mixed with ethylacetate and hexane. The precipitates were collected and dried to give 6-(benzyloxy)4-(dibenzylamino)-1-naphthaldehyde (45.1 g, 86% yield).

Next, to a mixture of 6-(benzyloxy)-4-(dibenzylamino)-1-naphthaldehyde (200.7 mg, 0.439 mmol) and 10% Pd/Carbon (54.0 mg) in methanol (10 mL) was stirred under high pressure hydrogen for 2 days. The mixture was passed through Celite, and the filtrate was concentrated under reduced pressure. The obtained residue was purified by column chromatography (silica gel, 1:1 hexane/ethylacetate) to give 8-amino-5-methyl-2-naphthol (173.2 mg, 88% yield).

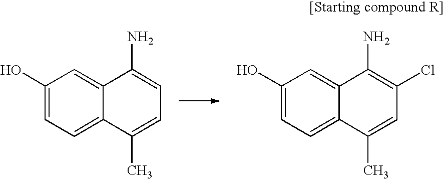

To a stirred solution of 8-amino-5-methyl-2-naphthol (150.0 mg, 0.87 mmol) in tetrahydrofuran (10 mL) was added N-chlorosuccinimide (115.6 mg, 0.87 mmol) at 0° C. The reaction mixture was stirred for 5 hours at room temperature, and the mixture was concentrated under reduced pressure. Ethylacetate was added to the mixture, and the organic layer was washed with water, dried over MgSO₄, filtered, and concentrated under reduced pressure. The obtained residue was triturated with dichloromethane and diisopropylether, filtered, and the filtrate was concentrated under reduced pressure to give 8-amino-7-chloro-5-methyl-2-naphthol (157.0 mg, 87%).

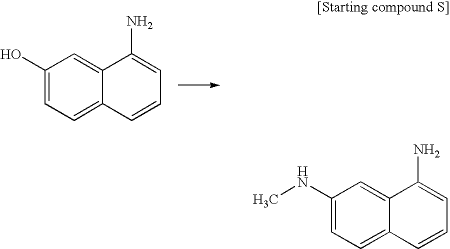

A stirred mixture of 8-amino-2-naphthol (1.00 g, 6.32 mmol) and 40% methylamine in water (10 mL) was stirred at 160° C. in a sealed tube for 2 days. After cooling to room temperature, the mixture was poured into water, and extracted with ethylacetate. The organic layer was washed with water, dried over MgSO₄, filtered, and concentrated under reduced pressure. The obtained residue was purified by column chromatography (silica gel, 1:3 hexane/ethylacetate) to give N-(8-amino-2-naphthyl)-N-methylamine (0.478 g, 44% yield).

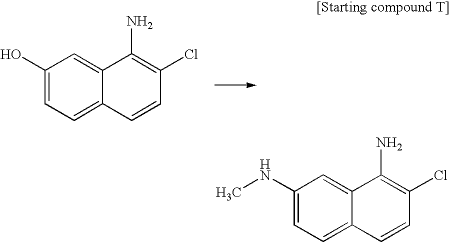

A stirred mixture of 8-amino-7-chloro-2-naphthol (195.0 mg, 1.01 mmol) and 40% methylamine in water (10 mL) was stirred at 180° C. in a sealed tube for 24 hours. After cooling to room temperature, the mixture was poured into water, and extracted with ethylacetate. The organic layer was washed with water, dried over MgSO₄, filtered, and concentrated under reduced pressure to give N-(8-amino-7-chloro-2-naphthyl)-N-methylamine (16.1 mg, 7.7% yield).

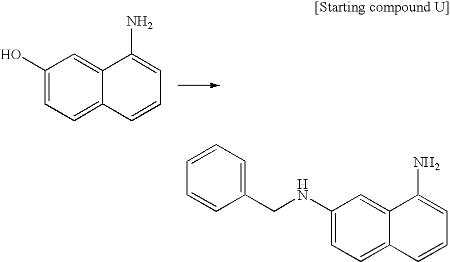

A stirred mixture of 8-amino-2-naphthol (1.10 g, 6.91 mmol) and benzylamine (1.61 g, 15.0 mmol) was stirred at 180 ° C. in a sealed tube for 2 days. After cooling to room temperature, the mixture was purified by column chromatography (silica gel, 1:2 hexane/ethylacetate) to give N-(8-amino-2-naphthyl)-N-benzylamine (1.39 g, 81% yield).

### Example 1-1

### N-(3-Chlorophenyl)-N′-(2,4-dichloro-7-hydroxy-1-naphthyl)urea

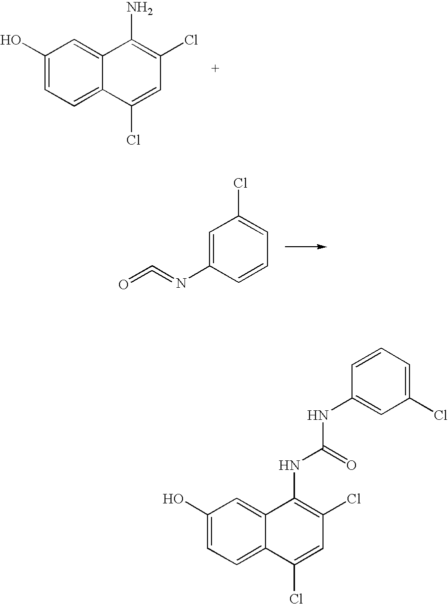

This example was performed according to the general method A.

A mixture of 8-amino-5,7-dichloro-2-naphthol (starting compound G) (100 mg, 0.438 mmol) and 3-chlorophenyl isocyanate (67.0 mg, 0.438 mmol) in 1,4-dioxane (5 mL) was stirred at 50° C. for 16 hours. The mixture was concentrated under reduced pressure, and to the residue was added isopropylether. The precipitate was filtered and dried to give N-(3-chlorophenyl)-N′-(2,4-dichloro-7-hydroxy-1-naphthyl)urea (65 mg, 39% yield).

Molecular weight 381.64.

MS (M+H):381

mp:>260° C.

With the use of any of the starting materials A-J , M-N, or Q-U and according to the similar procedure of Example 1-1, the following compounds were synthesized and tested. In the tables, Z stands for decomposition.

TABLE 1

| Ex. No | MOLSTRUCTURE | MW | MS | Melting Point (° C.) |
|---|---|---|---|---|
| 1-2 | 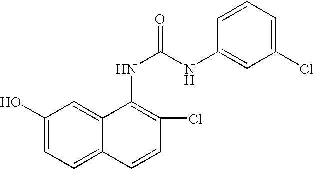 | 347, 2034 | 347 | 242-243 |
| 1-3 | 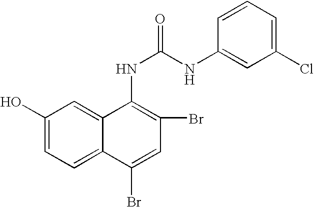 | 470, 5504 | 470 | 242-243 |
| 1-4 | 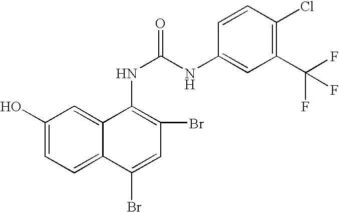 | 538, 5488 | 536 | 242-243 |
| 1-5 | 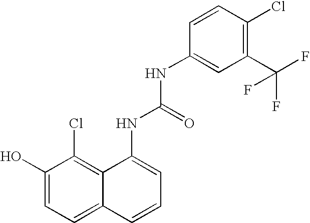 | 415, 2018 | 416 | >240 Z |
| 1-6 | 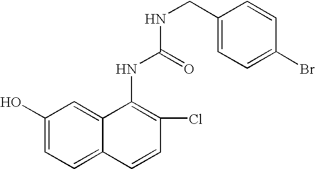 | 405, 6815 | 405, 407 | 226-229 |
| 1-7 | 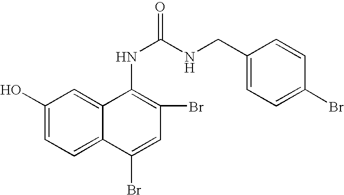 | 529, 0285 | | 215 Z |
| 1-8 | 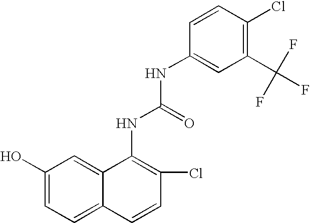 | 415, 2018 | 415 | 260-Z |
| 1-9 | 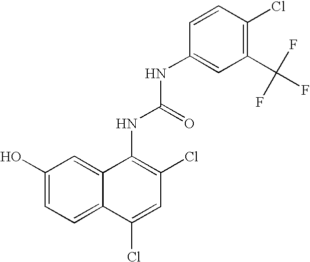 | 449, 6468 | 449 | 255-Z |
| 1-10 | 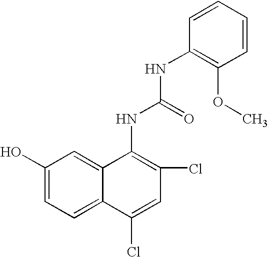 | 377, 2299 | 377, 379 | 251 Z |
| 1-11 | 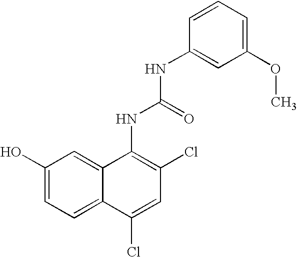 | 377, 2299 | 377 | 223-226 |
| 1-12 | 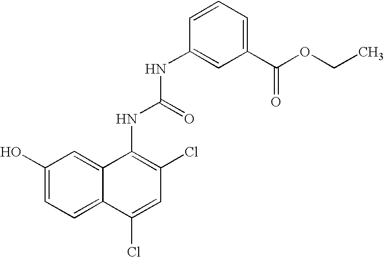 | 419, 2675 | 419 | 234-236 |
| 1-13 | 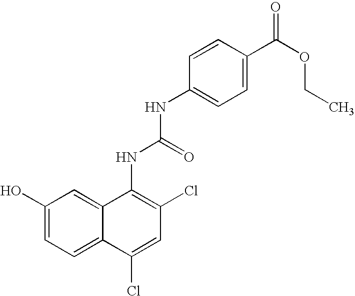 | 419, 2675 | 419, 421 | 258-260 |
| 1-14 | 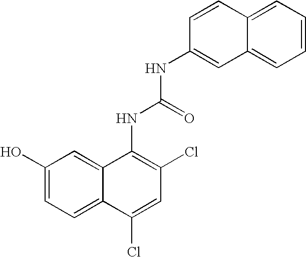 | 397, 2639 | 397, 399 | 263-265 |
| 1-15 | 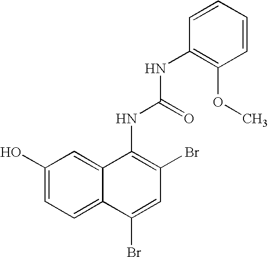 | 466, 1319 | 467, 469 | 228-230 |
| 1-16 | 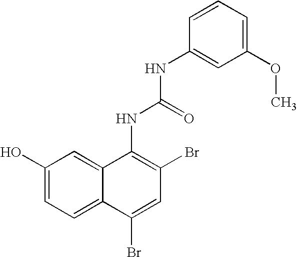 | 466, 1319 | 465, 467, 469 | 213-216 |
| 1-17 | 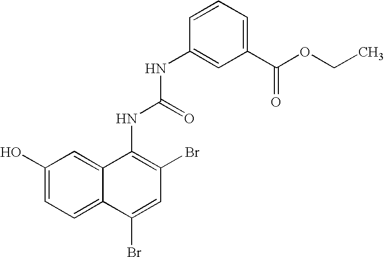 | 508, 1695 | 509 | 193-196 |
| 1-18 | 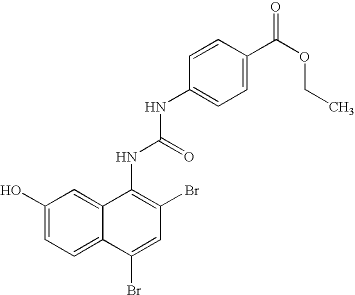 | 508, 1695 | 507, 509, 511 | 209 Z |
| 1-19 | 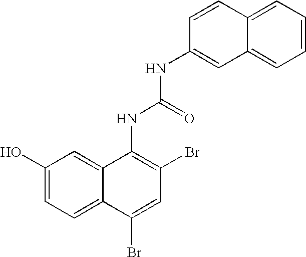 | 486, 1659 | nd | 195 Z |
| 1-20 | 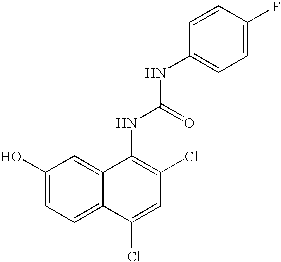 | 365, 1938 | 365, 367 | 250 Z |
| 1-21 | 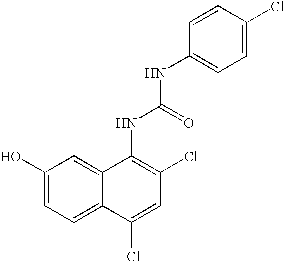 | 381, 6484 | 381,383 | 253-255 |
| 1-22 | 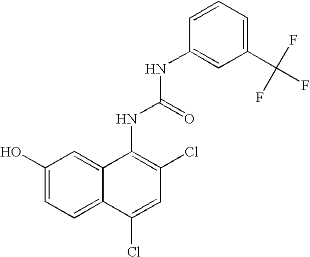 | 415, 2018 | 415 | 262 Z |
| 1-23 | 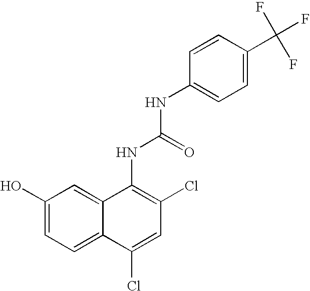 | 415, 2018 | 415, 417 | 268-271 |
| 1-24 | 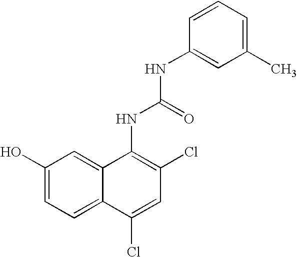 | 361, 2305 | 361, 363 | 223 Z |
| 1-25 | 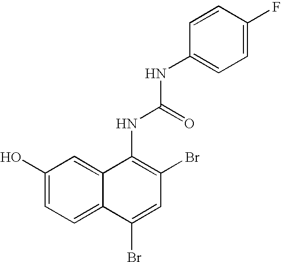 | 454, 0958 | 453, 455, 457 | 222-225 |
| 1-26 | 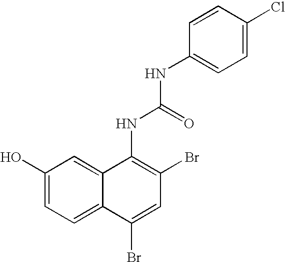 | 470, 5504 | 469, 471, 473 | 229-233 |
| 1-27 | 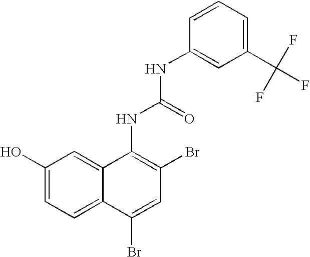 | 504, 1038 | 503, 505, 507 | 233-236 |
| 1-28 | 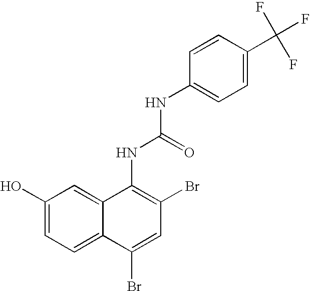 | 504, 1038 | 503, 505, 507 | 229 Z |
| 1-29 | 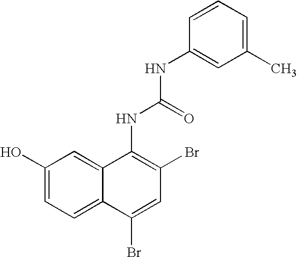 | 450, 1325 | 451 | 164 Z |
| 1-30 | 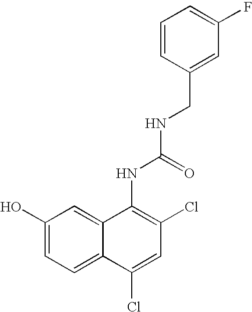 | 379, 2209 | 379, 381 | 225-228 |
| 1-31 | 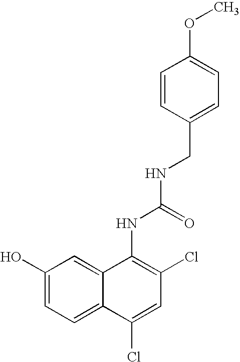 | 391, 257 | 391 | 223-226 |
| 1-32 | 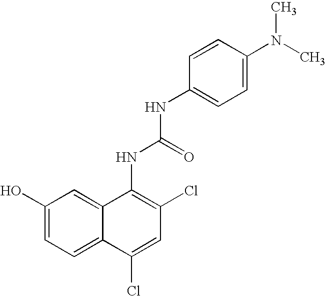 | 390, 2722 | 390 | 192-Z |
| 1-33 | 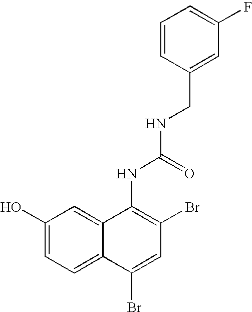 | 468, 1229 | 467, 469, 471 | 215-218 |
| 1-34 | 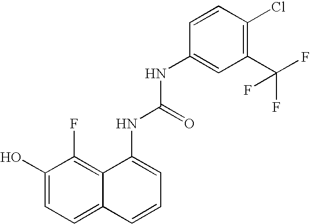 | 398, 7472 | 399 | 228 |
| 1-35 | 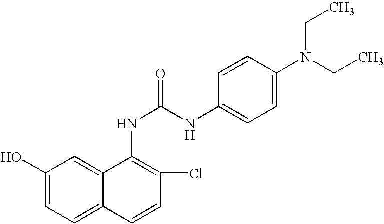 | 383, 8814 | | 199.8-200.5 |
| 1-36 | 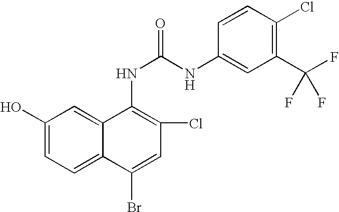 | 494, 0978 | | 209 Z |
| 1-37 | 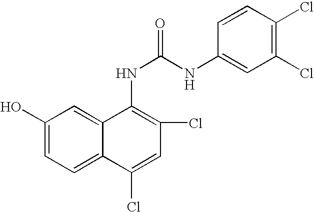 | 416, 0935 | 415 | 249 Z |
| 1-38 | 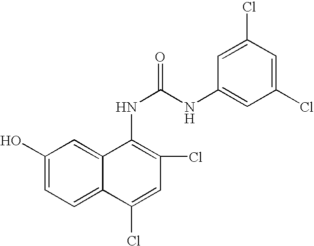 | 416, 0935 | 415 | 265 Z |
| 1-39 | 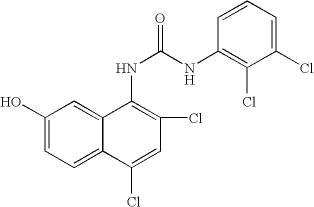 | 416, 0935 | 415 | 300 |
| 1-40 | 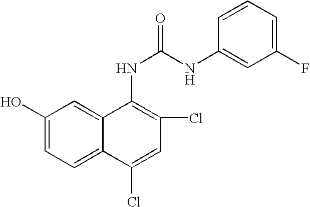 | 365, 1938 | 365 | >300 |
| 1-41 | 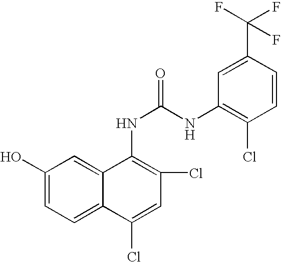 | 449, 6468 | 449 | >300 |
| 1-42 | 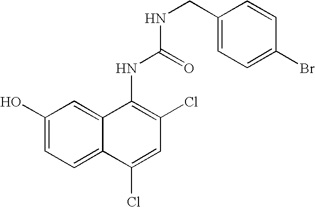 | 440, 1265 | 439 | 226 Z |
| 1-43 | 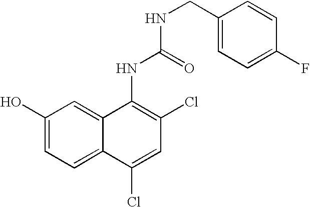 | 379, 2209 | 379 | 229 Z |
| 1-44 | 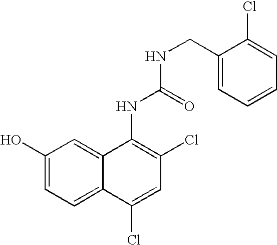 | 395, 6755 | 395 | 240 Z |
| 1-45 | 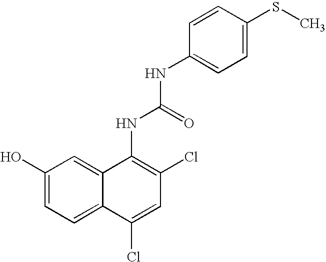 | 393, 2945 | 393 | >231 Z |
| 1-46 | 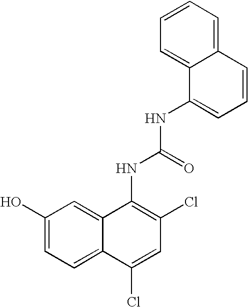 | 397, 2639 | 397 | 269 Z |
| 1-47 | 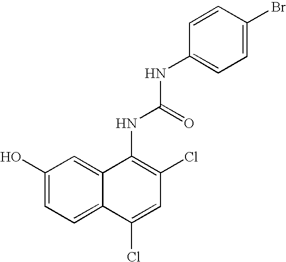 | 426, 0994 | 424 | 258 Z |
| 1-48 | 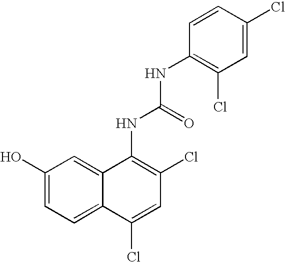 | 416, 0935 | nd | 286 Z |
| 1-49 | 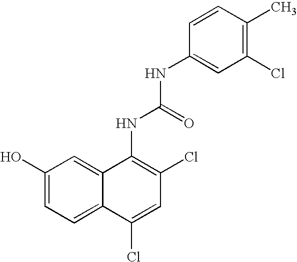 | 395, 6755 | 395 | 248 Z |
| 1-50 | 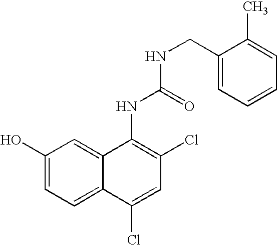 | 375, 2576 | 375 | 239 Z |
| 1-51 | 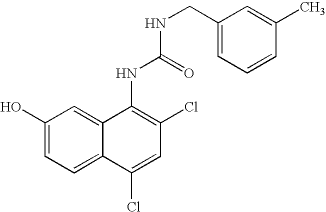 | 375, 2576 | 375 | 227 Z |
| 1-52 | 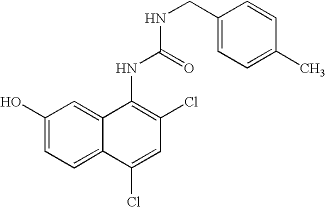 | 375, 2576 | 375 | 224 Z |
| 1-53 | 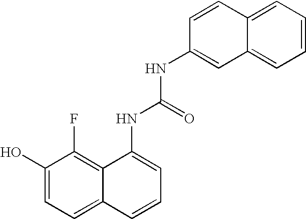 | 346, 3643 | 347 | 189 |
| 1-54 | 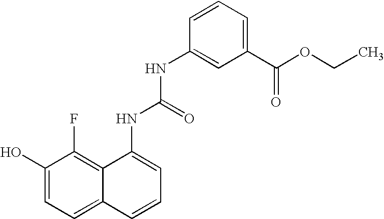 | 368, 3679 | 370 | 174 |
| 1-55 | 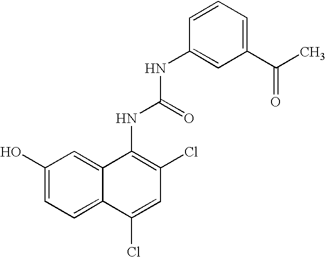 | 389, 241 | 389 | 223 Z |
| 1-56 | 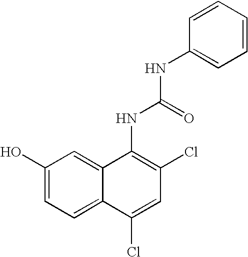 | 347, 2034 | 347 | 245 Z |
| 1-57 | 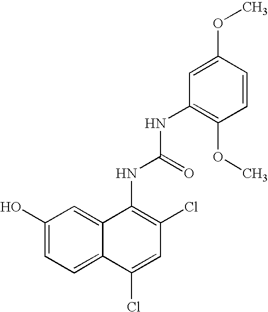 | 407, 2564 | 407 | 258 Z |
| 1-58 | 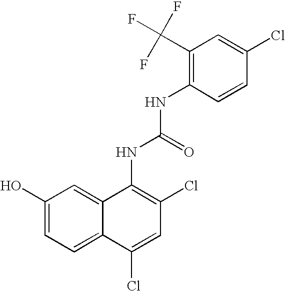 | 449, 6468 | 449 | 283 Z |
| 1-59 | 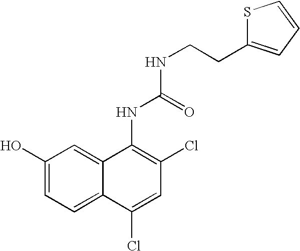 | 381, 2833 | 381 | 234 Z |
| 1-60 | 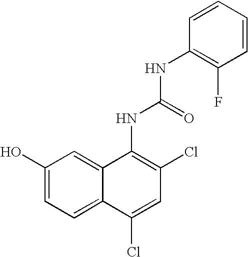 | 365, 1938 | 365 | 297 Z |
| 1-61 | 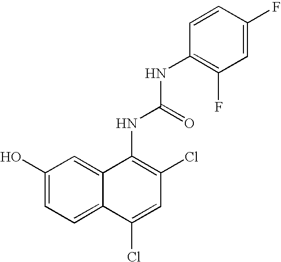 | 383, 1843 | 383 | 300 Z |
| 1-62 | 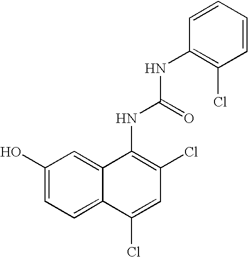 | 381, 6484 | 381 | 250 Z |
| 1-63 | 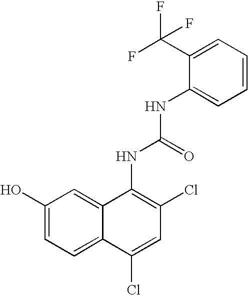 | 415, 2018 | 415 | 300 Z |
| 1-64 | 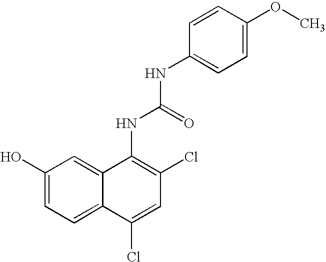 | 377, 2299 | 377 | 243 Z |
| 1-65 | 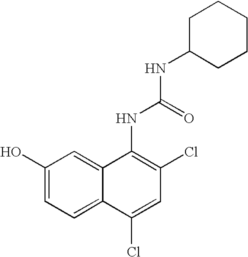 | 353, 2512 | 353 | 217 Z |
| 1-66 | 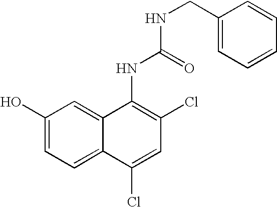 | 361, 2305 | 361 | 220 Z |
| 1-67 | 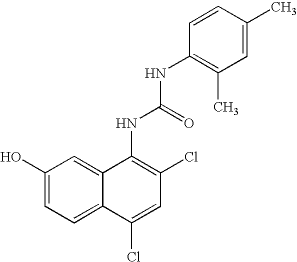 | 375, 2576 | 375 | 254 Z |
| 1-68 | 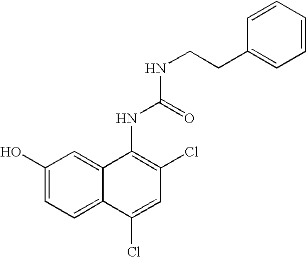 | 375, 2576 | 375 | 235 Z |
| 1-69 | 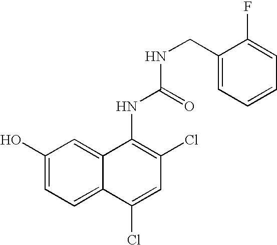 | 379, 2209 | 379 | 218 Z |
| 1-70 | 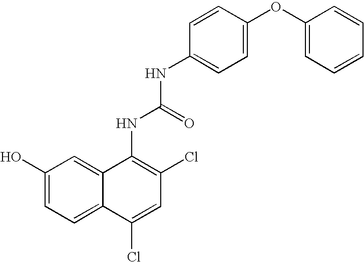 | 439, 3016 | 439 | 230 Z |
| 1-71 | 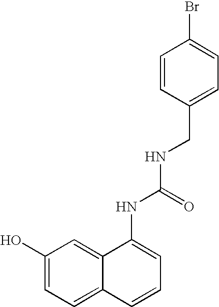 | 389, 2269 | 390 | 210 |
| 1-72 | 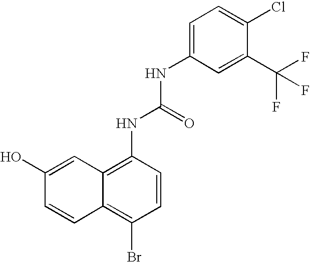 | 459, 6528 | — | 211 |
| 1-73 | 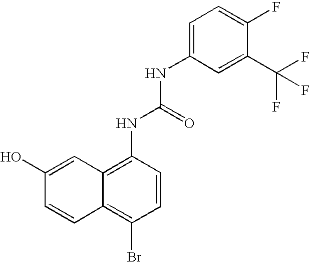 | 443, 1982 | | 215 |
| 1-74 | 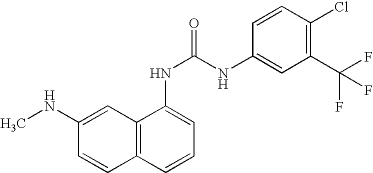 | 393, 7991 | 394 | 218-219 |
| 1-75 | 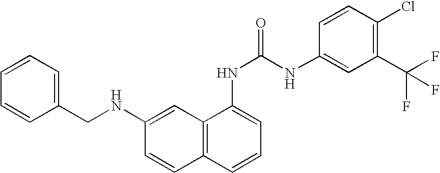 | 469, 8979 | 470 | 193-194 |
| 1-76 | 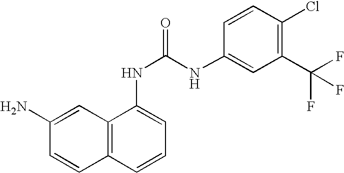 | 379, 772 | 380 | 232-234 |
| 1-77 | 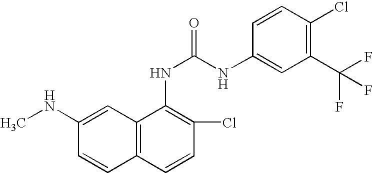 | 428, 2441 | 429 | 258-259 |
| 1-78 | 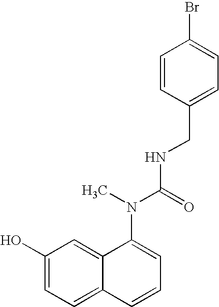 | 385, 2635 | 386 | 194 |
| 1-79 | 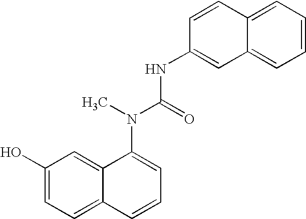 | 342, 401 | 343 | 215 |
| 1-80 | 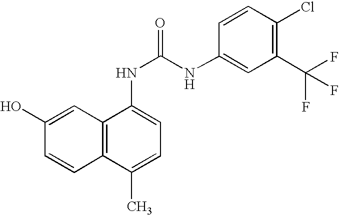 | 394, 7838 | 395 | 237-238 |
| 1-81 | 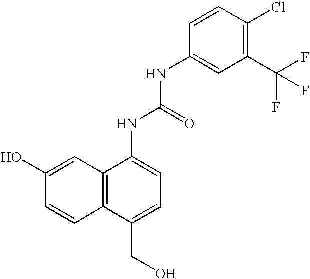 | 410, 7832 | 411 | 201 Z |
| 1-82 | 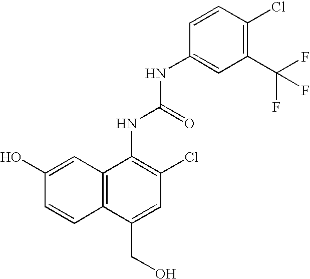 | 445, 2283 | 446 | 210 |
| 1-83 | 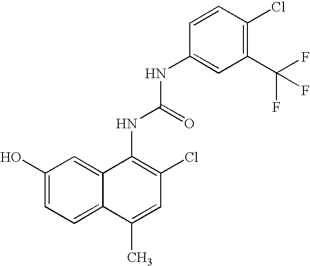 | 429, 2289 | 430 | 254 |
| 1-84 | 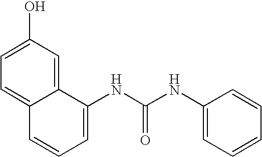 | 278, 3133 | 279 | |
| 1-85 | 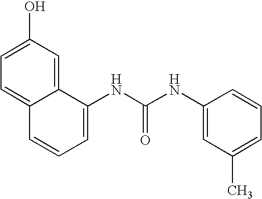 | 292, 3404 | 293 | |
| 1-86 | 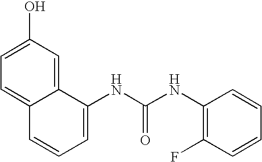 | 296, 3038 | 297 | |
| 1-87 | 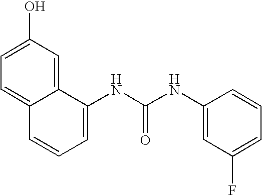 | 296, 3038 | 297 | |
| 1-88 | 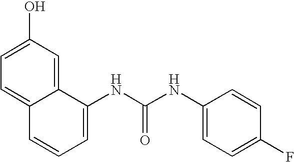 | 296, 3038 | 297 | |
| 1-89 | 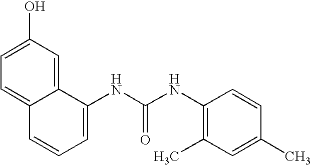 | 306, 3675 | 307 | |
| 1-90 | 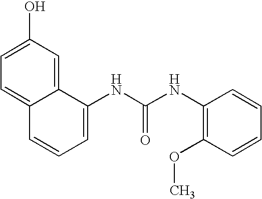 | 308, 3398 | 309 | |
| 1-91 | 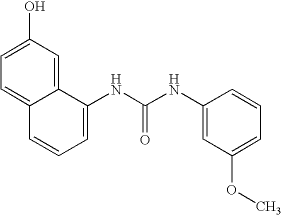 | 308, 3398 | 309 | |
| 1-92 | 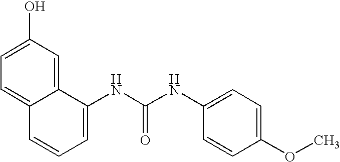 | 308, 3398 | 309 | |
| 1-93 | 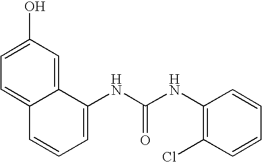 | 312, 7584 | 313 | |
| 1-94 | 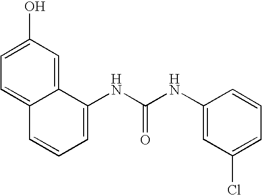 | 312, 7584 | 313 | |
| 1-95 | 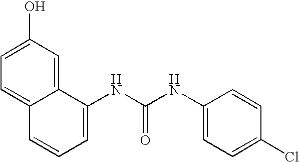 | 312, 7584 | 313 | |
| 1-96 | 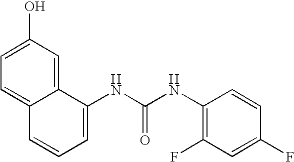 | 314, 2942 | 315 | |
| 1-97 | 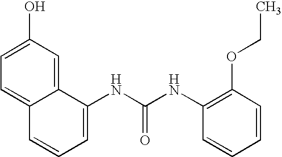 | 322, 3669 | 323 | |
| 1-98 | 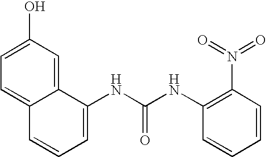 | 323, 3109 | 324 | |
| 1-99 | 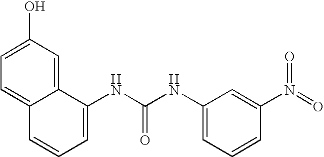 | 323, 3109 | 324 | |
| 1-100 | 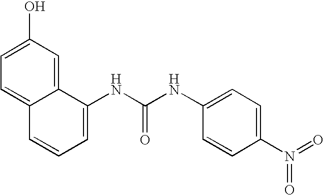 | 323, 3109 | 324 | |
| 1-101 | 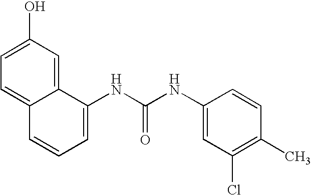 | 326, 7855 | 327 | |
| 1-102 | 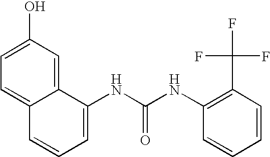 | 346, 3117 | 347 | |
| 1-103 | 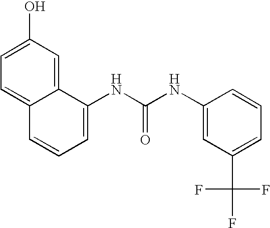 | 346, 3117 | 347 | |
| 1-104 | 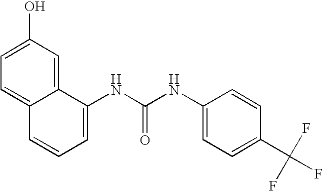 | 346, 3117 | 347 | |
| 1-105 | 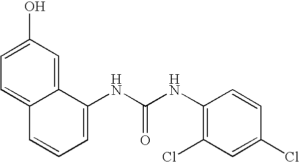 | 347, 2034 | 348 | |
| 1-106 | 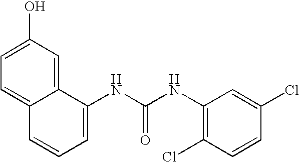 | 347, 2034 | 348 | |
| 1-107 | 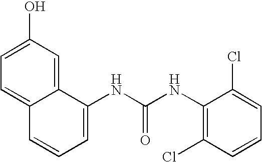 | 347, 2034 | 348 | |
| 1-108 | 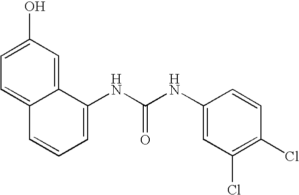 | 347, 2034 | 348 | |
| 1-109 | 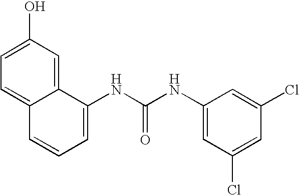 | 347, 2034 | 348 | |
| 1-110 | 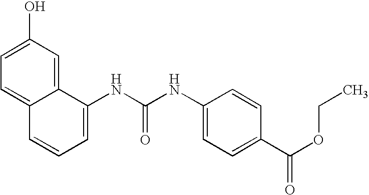 | 350, 3775 | 351 | |
| 1-111 | 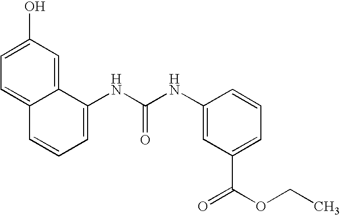 | 350, 3775 | 351 | |
| 1-112 | 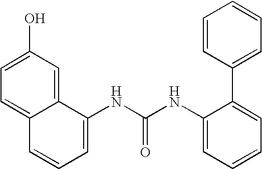 | 354, 4121 | 355 | |
| 1-113 | 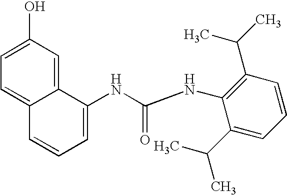 | 362, 4759 | 363 | |
| 1-114 | 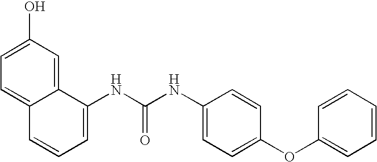 | 370, 4115 | 371 | |
| 1-115 | 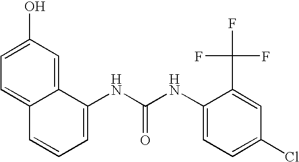 | 380, 7567 | 381 | |
| 1-116 | 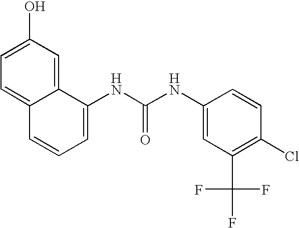 | 380, 7567 | 381 | |
| 1-117 | 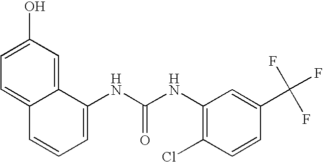 | 380, 7567 | 381 | |
| 1-118 | 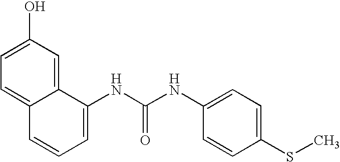 | 324, 4044 | 325 | |
| 1-119 | 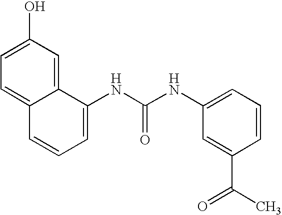 | 320, 351 | 321 | |
| 1-120 | 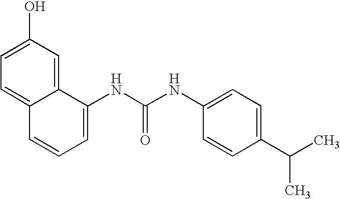 | 320, 3946 | 321 | |
| 1-121 | 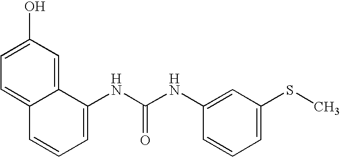 | 324, 4044 | 325 | |
| 1-122 | 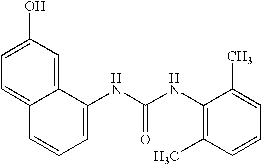 | 306, 3675 | 307 | |
| 1-123 | 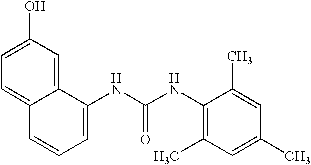 | 320, 3946 | 321 | |
| 1-124 | 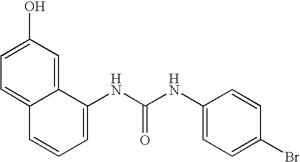 | 357, 2094 | 358 | |
| 1-125 | 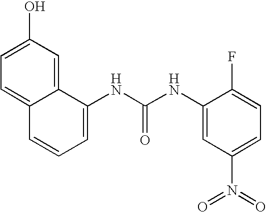 | 341, 3013 | 342 | |
| 1-126 | 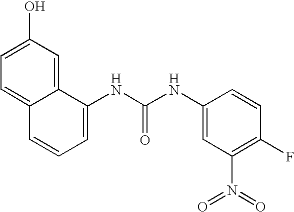 | 341, 3013 | 342 | |
| 1-127 | 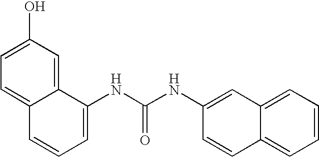 | 328, 3739 | 329 | |
| 1-128 | 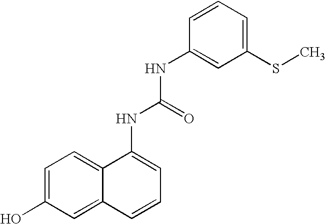 | 324, 4044 | nd | |
| 1-129 | 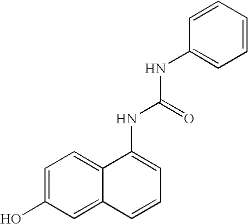 | 278, 3133 | 279 | |
| 1-130 | 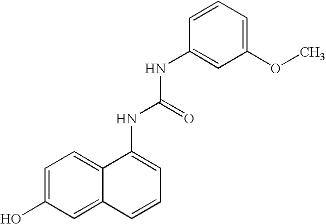 | 308, 3398 | 309 | |
| 1-131 | 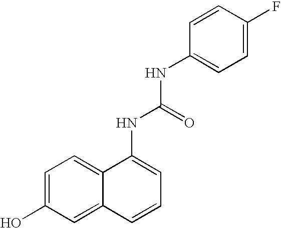 | 296, 3038 | nd | |
| 1-132 | 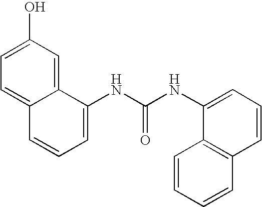 | 328, 3739 | 329 | |
| 1-133 | 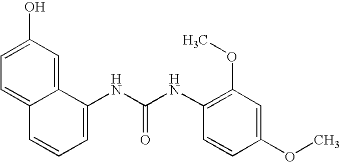 | 338, 3663 | 339 | |
| 1-134 | 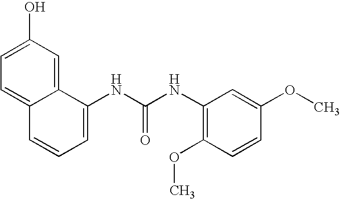 | 338, 3663 | 339 | |
| 1-135 | 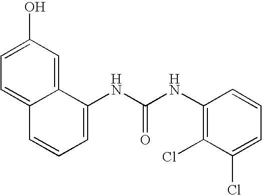 | 347, 2034 | 348 | |
| 1-136 | 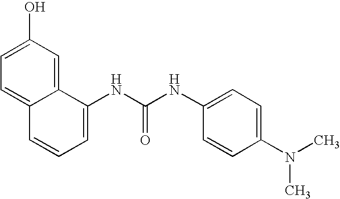 | 321, 3822 | 322 | |
| 1-137 | 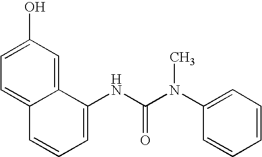 | 292, 3404 | 293 | |
| 1-138 | 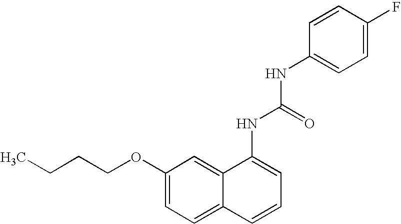 | 352, 4121 | 353 | |
| 1-139 | 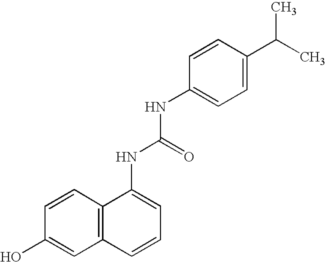 | 320, 3946 | 321 | 207.5 |
| 1-140 | 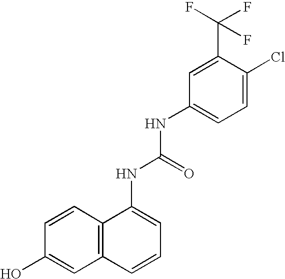 | 380, 7567 | 381 | |
| 1-141 | 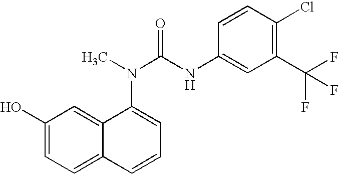 | 394, 7838 | 395 | |
| 1-142 | 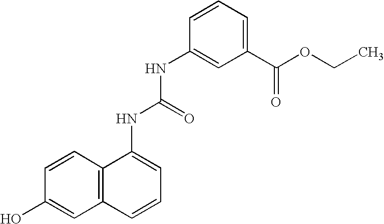 | 350, 3775 | 351 | |
| 1-143 | 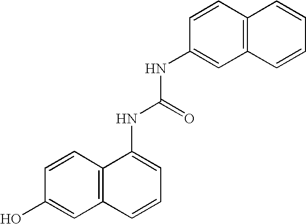 | 328, 3739 | 329 | |
| 1-144 | 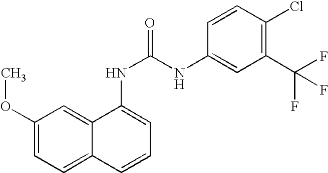 | 394, 7838 | 395 | |
| 1-145 | 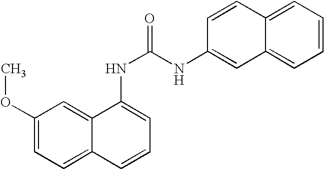 | 342, 401 | 343 | |
| 1-146 | 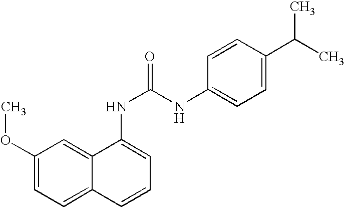 | 334, 4217 | 335 | |
| 1-147 | 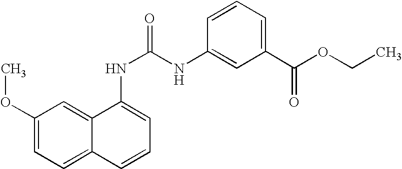 | 364, 4046 | 365 | |
| 1-148 | 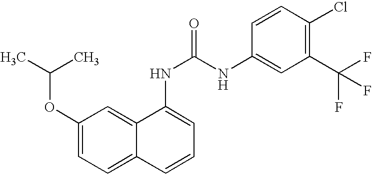 | 422, 838 | 423 | |
| 1-149 | 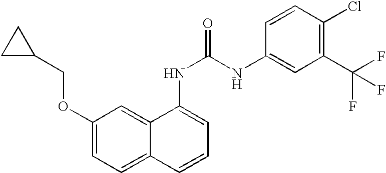 | 434, 8492 | 435 | |
| 1-150 | 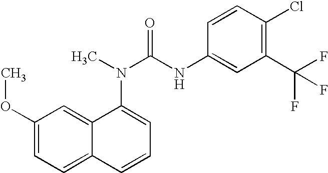 | 408, 8109 | 409 | |
| 1-151 | 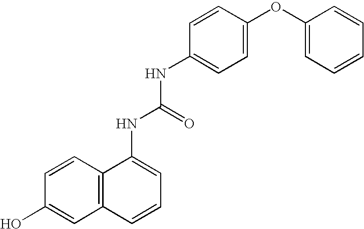 | 370, 4115 | 371 | |
| 1-152 | 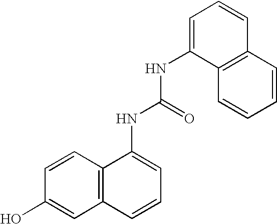 | 328, 3739 | 329 | |
| 1-153 | 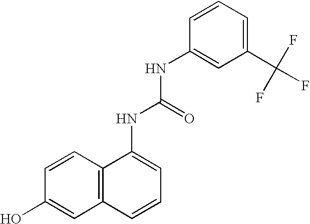 | 346, 3117 | 347 | |
| 1-154 | 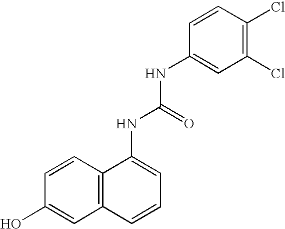 | 347, 2034 | 347 | |
| 1-155 | 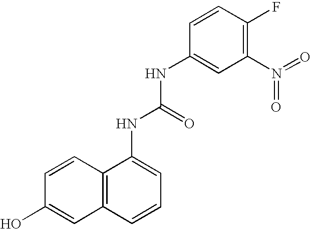 | 341, 3013 | 342 | |
| 1-156 | 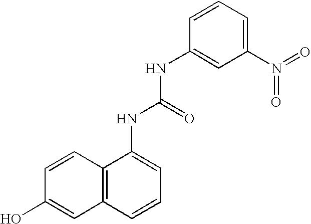 | 323, 3109 | 324 | |
| 1-157 | 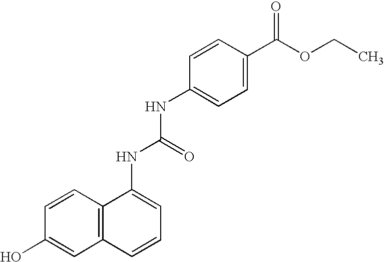 | 350, 3775 | 351 | |
| 1-158 | 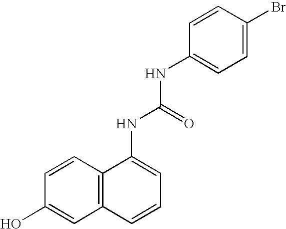 | 357, 2094 | 359 | |
| 1-159 | 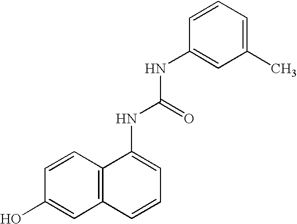 | 292, 3404 | 293 | |
| 1-160 | 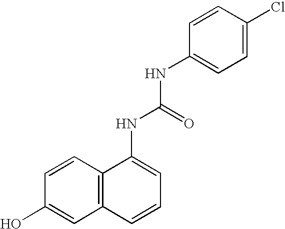 | 312, 7584 | 313 | |
| 1-161 | 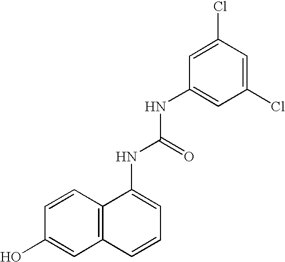 | 347, 2034 | 347 | |
| 1-162 | 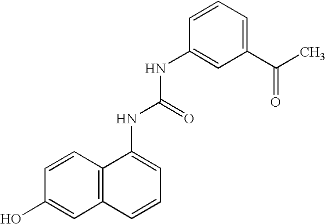 | 320, 351 | 321 | |
| 1-163 | 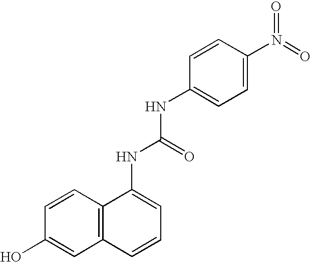 | 323, 3109 | 324 | |
| 1-164 | 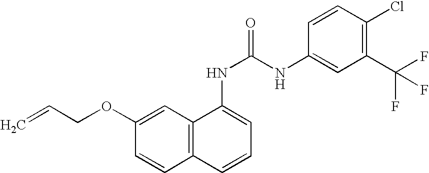 | 420, 8221 | 421 | 183-184 |
| 1-165 | 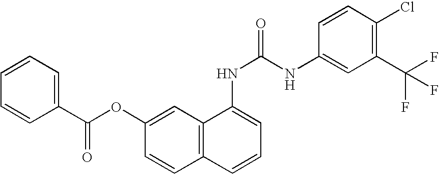 | 484, 8661 | 485 | 220-222 |
| 1-166 | 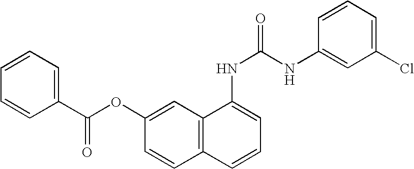 | 416, 8677 | 417 | 214-215 |
| 1-167 | 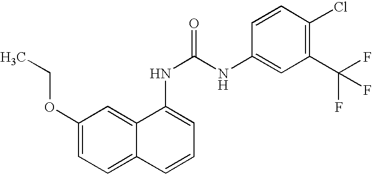 | 408, 8109 | 409 | |
| 1-168 | 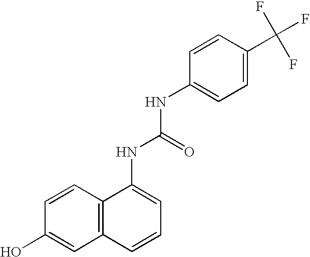 | 346, 3117 | 347 | |
| 1-169 | 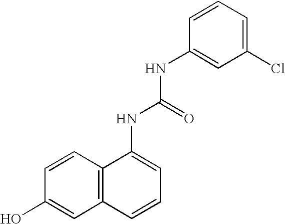 | 312, 7584 | 313 | |
| 1-170 | 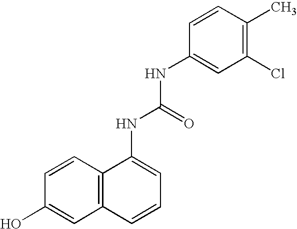 | 326, 7855 | 327 | |
| 1-171 | 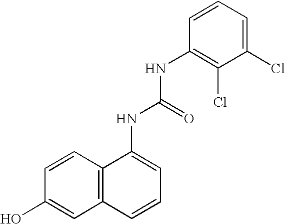 | 347, 2034 | 347 | |
| 1-172 | 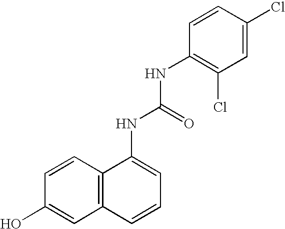 | 347, 2034 | 347 | |
| 1-173 | 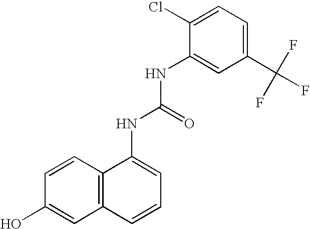 | 380, 7567 | 381 | |
| 1-174 | 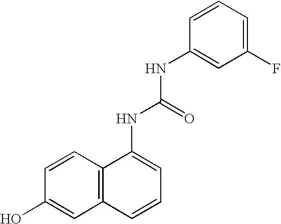 | 296, 3038 | 297 | |
| 1-175 | 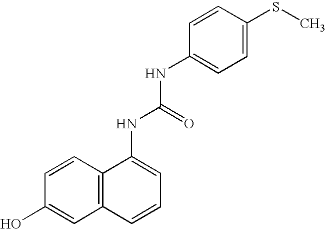 | 324, 4044 | 325 | |
| 1-176 | 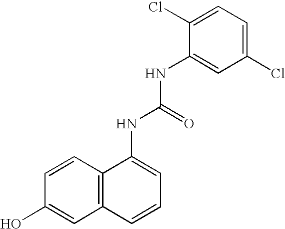 | 347, 2034 | 347 | |
| 1-177 | 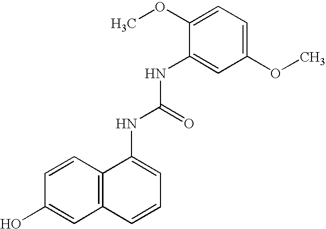 | 338, 3663 | 339 | |
| 1-178 | 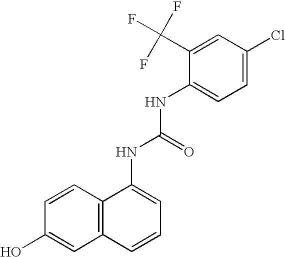 | 380, 7567 | 381 | |

### Example 2-1

### N-(1,1′-Biphenyl-3-yl)-N′-(2-chloro-7-hydroxy-naphthyl)urea

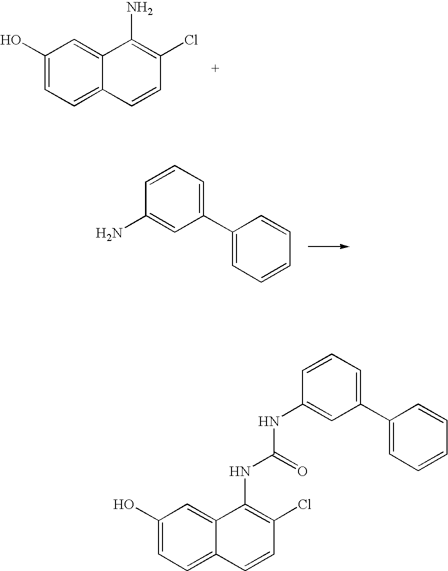

This example was performed according to the general method B.

To the solution of 8-amino-7-chloro-2-naphthol(starting compound F) (67.77 mg, 0.35 mmol) and pyridine (0.04 mL, 0.44 mmol) in THF (1 mL) was added phenyl chloroformate (57.93 mg, 0.37 mmol) at room temperature. The mixture was stirred for 1 hour at room temperature. To the reaction mixture was added ethylacetate and washed with water and brine. The organic layer was concentrated in vacuo. To the residue was added DMSO (1 mL) and then added a 3-aminobiphenyl at room temperature. The mixture was stirred for 16 hours at 100° C. To the mixture was added water, and the precipitate was filtered and washed with diisopropyl ether to give N-(1, l′-biphenyl-3-yl)-N′-(2-chloro-7-hydroxy-1-naphthyl)urea (102.1 mg, 87.5%).

Molecular weight 388.86.

MS (M+H):389.

mp: 234236° C.

With the use of the starting material F and according to the similar procedure of Example 2-1, the following compound was synthesized and tested.

TABLE 2

| Ex. No. | MOL STRUCTURE | MW | MS | Melting Point |
|---|---|---|---|---|
| 2-2 | 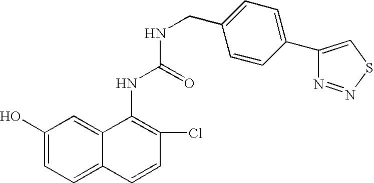 | 410.89 | nd | 241-244 |

### Example 3-1

### 5,7-Dichloro-8-({[(2′-chloro-1,1′-biphenyl-3-yl)amino]carbonyl}amino)-2-naphthyl acetate

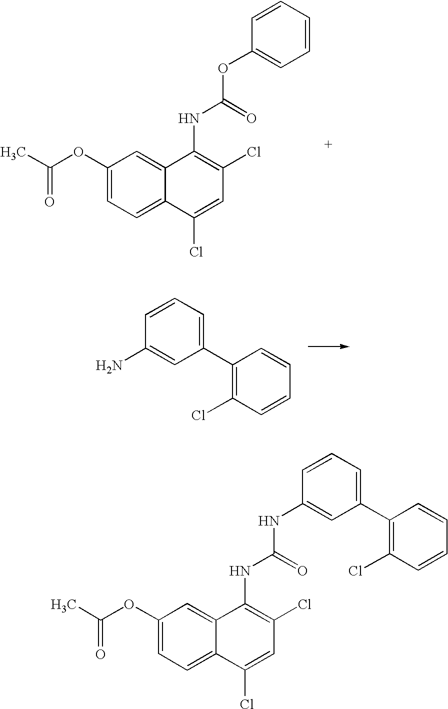

This example was performed according to the general method C.

A mixture of 5,7-dichloro-8-[(phenoxycarbonyl)amino]-2-naphthyl acetate (starting compound K) (762 mg, 2.0 mmol) and 2′-chloro-biphenyl-3-ylamine (407 mg, 2.0 mmol) in DMSO (6 mL) was stirred for 5 hours at 100° C. To the reaction mixture was added water, the precipitate was filtered and dried to give acetic acid 5,7-dichloro-8-({[(2′-chloro-1,1′-biphenyl-3-yl)amino]carbonyl}amino)-2-naphthyl acetate (805 mg, 81%).

Molecular weight 499.78.

mp: 180° C.

With the use of the starting material K and according to the similar procedure of Example 3-1, the following compounds were synthesized and tested.

TABLE 3

| Ex. No | MOLSTRUCTURE | MW | MS | Melting Point (° C.) |
|---|---|---|---|---|
| 3-2 | 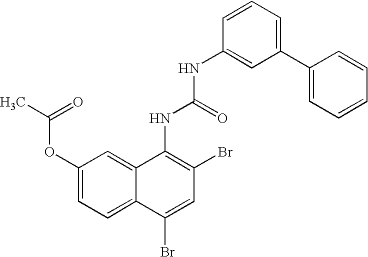 | 554.24181 | 555 | 235-Z |
| 3-3 | 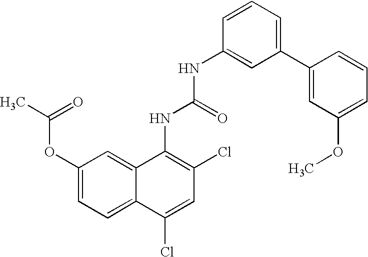 | 495.3663 | 495,497 | 224 Z |
| 3-4 | 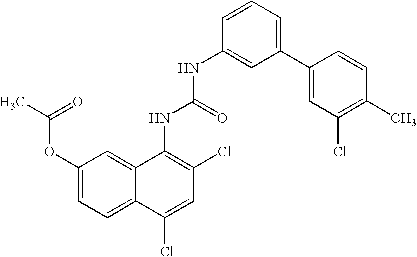 | 513.81193 | 513,515 | 260 |
| 3-5 | 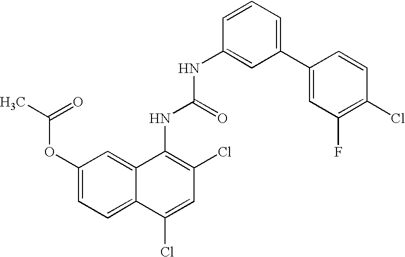 | 517.77527 | 517,519 | 287 |
| 3-6 | 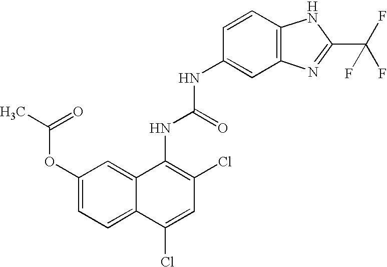 | 497.26396 | 497 | 210 Z |
| 3-7 | 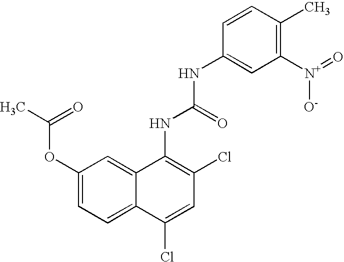 | 448.26565 | 448 | 210 Z |
| 3-8 | 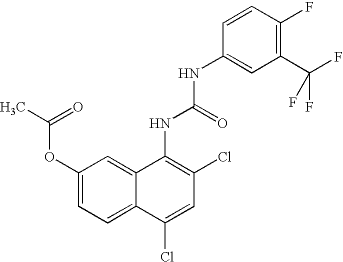 | 475.22984 | 475 | 209 Z |
| 3-9 | 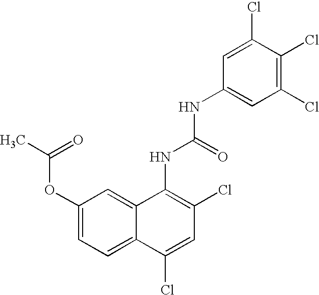 | 492.57612 | 491 | 235 Z |
| 3-10 | 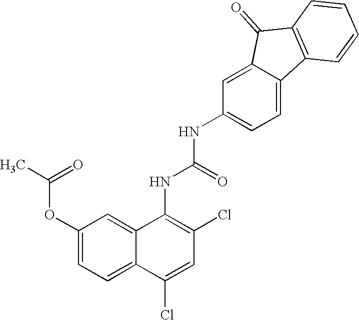 | 491.33442 | 491,493 | 213-Z |
| 3-11 | 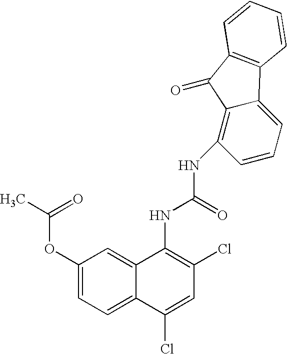 | 491.33442 | 491 | ND |
| 3-12 | 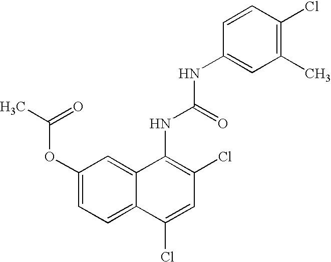 | 437.71315 | 437 | ND |
| 3-13 | 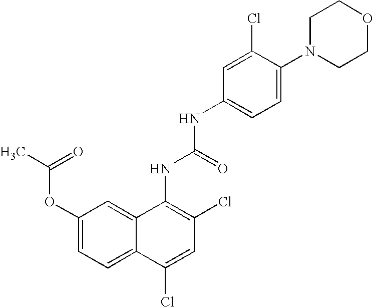 | 508.79255 | 508,510 | 206 |
| 3-14 | 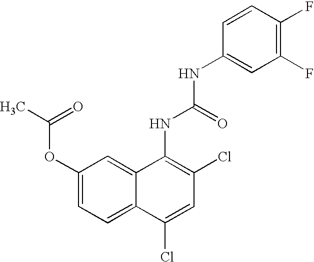 | 425.22189 | 425,427 | 226-Z |
| 3-15 | 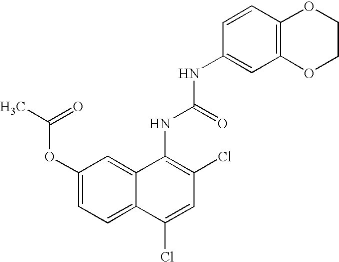 | 447.27807 | nd | 240 Z |
| 3-16 | 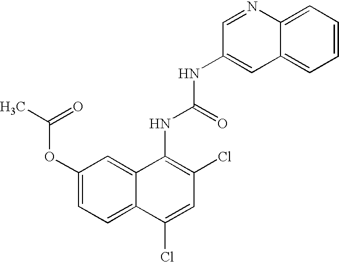 | 440.28915 | 440,442 | 205-Z |
| 3-17 | 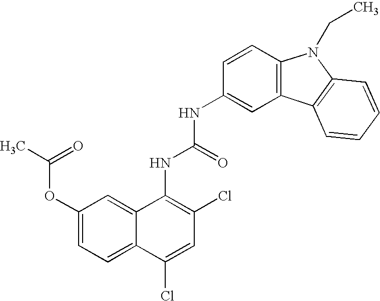 | 506.39272 | 506 | 260 Z |

### Example 4-1

### N-(2,4-Dichloro-7-hydroxy-1-naphthyl)-N′-(4-propylphenyl)urea

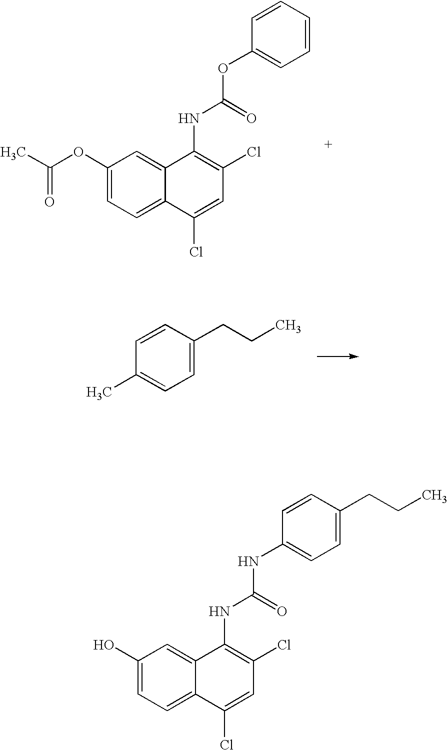

This example was performed according to the general method D.

(1) A mixture of 5,7-dichloro-8-[(phenoxycarbonyl)amino]-2-naphthyl acetate (starting compound K) (195.11 mg, 0.5 mmol) and 4-propylaniline (67.61 mg, 0.5 mmol) in DMSO (1.5 mL) was stirred for 5 hours at 100° C. To the reaction mixture was added water, the precipitate was filtered and dried to give 5,7-dichloro-8-({[(4-propylphenyl)amino]carbonyl}amino)-2-naphthyl acetate (88.4 mg, 41%).

(2)

Next, a mixture of 5,7-dichloro-8-({[(4-propylphenyl)amino]carbonyl}-amino)-2-naphthyl acetate (88.0 mg, 0.2 mmol) and potassium carbonate (207 mg) in methanol (6mL) was heated at 50° C. for 14 hours. After filtration, the mixture was concentrated in vacuo. The residue was washed with water, filtrated, and dried. To the obtained solid was added Dowex (492 mg) and methanol (4 mL), and the mixture was heated at 50° C. for 3 hours. To the mixture was added acetone and then filtrated. After washed with acetone, the filtrate was concentrated in vacuo. The residue was washed with diisopropyl ether to give N-(2,4dichloro-7-hydroxy-1-naphthyl)-N′-(4-propylphenyl)urea (52.7 mg, 66%/0).

Molecular weight 389.28.

MS (M+H):389.

mp: 241° C.

With the use of the starting material K and according to the similar procedure of Example 4-1 (1) to (3), or (1) to (2) (potassium salts), the following compounds were synthesized and tested.

TABLE 4

| Ex. No | MOLSTRUCTURE | MW | MS | Melting Point (° C.) |
|---|---|---|---|---|
| 4-2 | 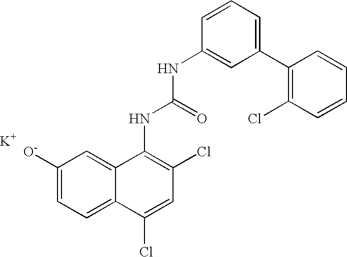 | 495.84123 | nd | 209 Z |
| 4-3 | 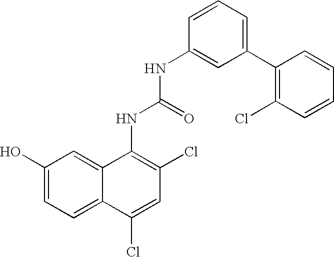 | 457.7472 | 457 | 228-232 |
| 4-4 | 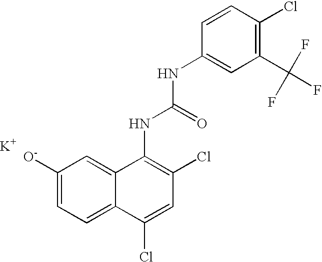 | 487.74083 | nd | 150-Z |
| 4-5 | 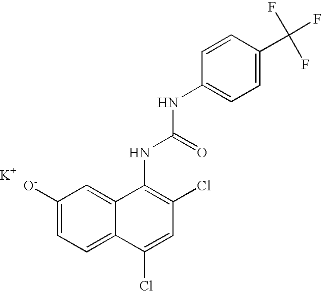 | 453.2958 | nd | 179-Z |
| 4-6 | 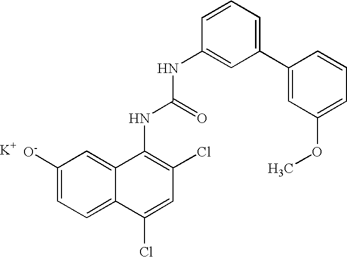 | 491.42269 | 453,455 | 206-Z |
| 4-7 | 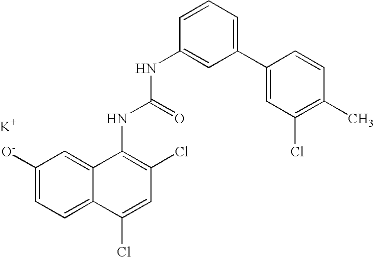 | 509.86832 | 511 | 203-Z |
| 4-8 | 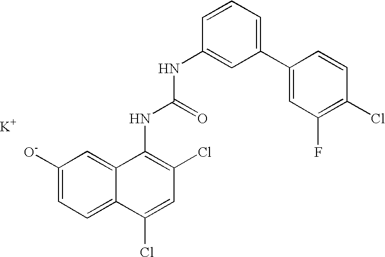 | 513.83166 | 470,472 | 174-Z |
| 4-9 | 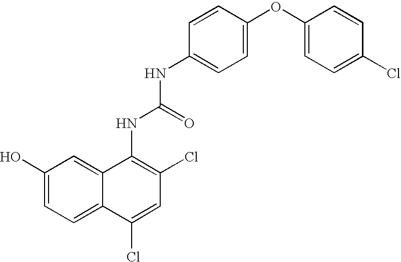 | 473.7466 | nd | 230 |
| 4-10 | 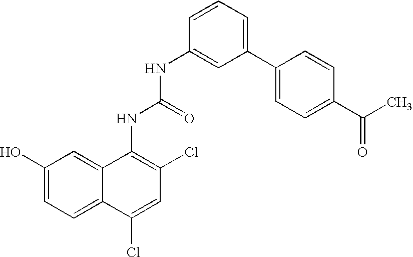 | 465.33981 | nd | 253 |
| 4-11 | 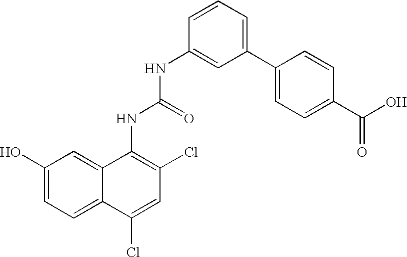 | 467.31212 | nd | 247-Z |
| 4-12 | 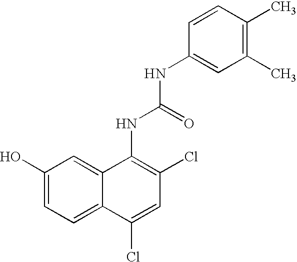 | 375.25757 | 375,377 | 239-Z |
| 4-13 | 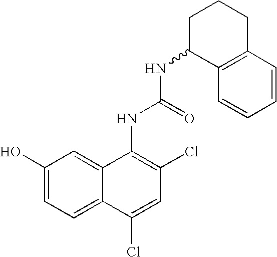 | 401.29581 | nd | 238-Z |
| 4-14 | 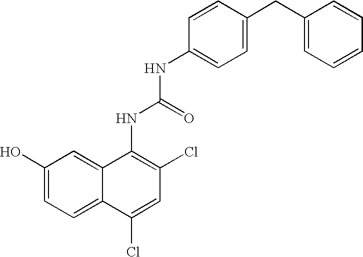 | 437.32926 | 437,439 | 230-Z |
| 4-15 | 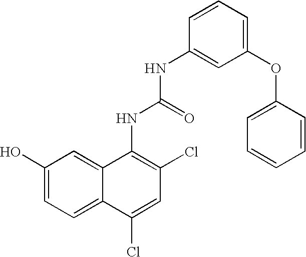 | 439.30157 | 439 | 226-Z |
| 4-16 | 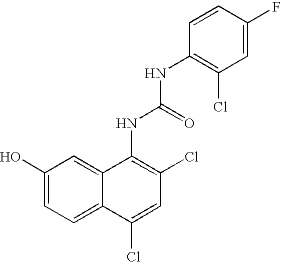 | 399.63885 | 399 | 298-Z |
| 4-17 | 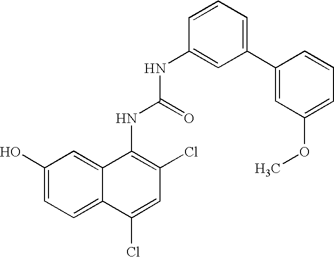 | 453.32866 | nd | 246-Z |
| 4-18 | 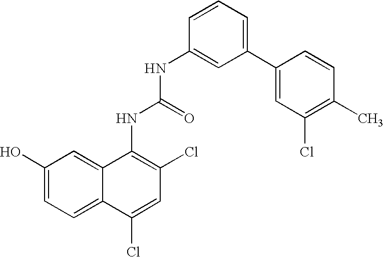 | 471.77429 | nd | 234-Z |
| 4-19 | 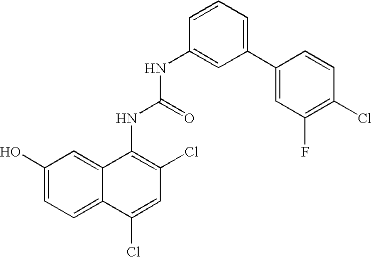 | 475.73763 | nd | 241-Z |
| 4-20 | 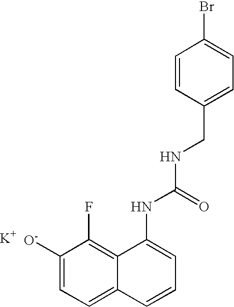 | 427.32091 | — | 185 |
| 4-21 | 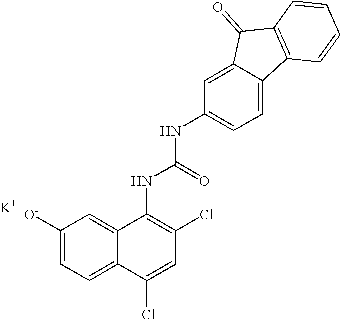 | 487.39081 | 449,451 | 200 |
| 4-22 | 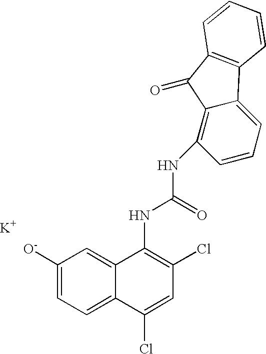 | 487.39081 | 449,451 | 195 |
| 4-23 | 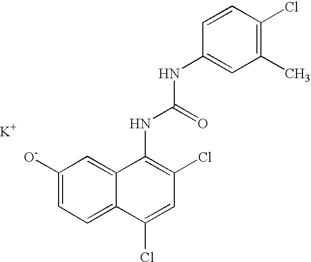 | 433.76954 | 395,397 | 190 |
| 4-24 | 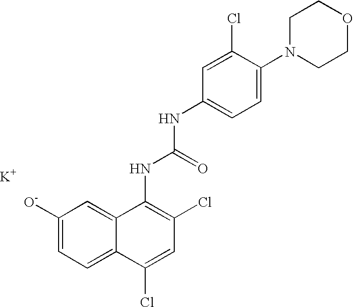 | 504.84894 | 466,468 | 188 |
| 4-25 | 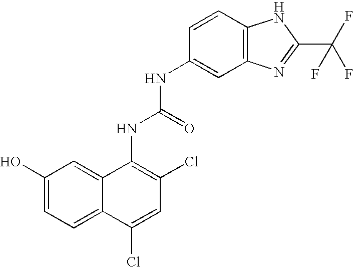 | 455.22632 | 455 | ND |
| 4-26 | 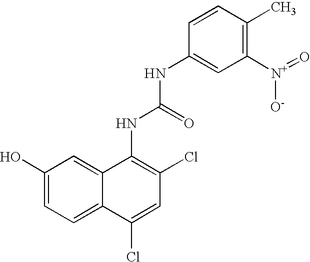 | 406.22801 | 406 | 250 Z |
| 4-27 | 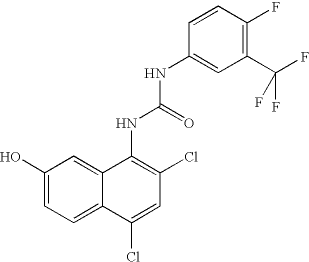 | 433.1922 | 433 | 228 Z |
| 4-28 | 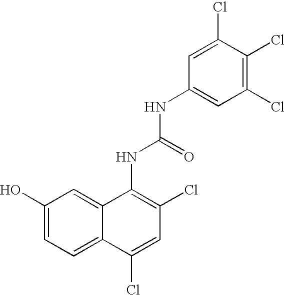 | 450.53848 | nd | 251 Z |
| 4-29 | 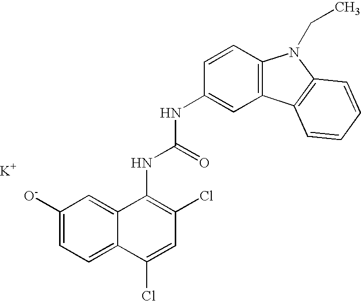 | 502.44911 | 464(free) | 188 Z |
| 4-30 | 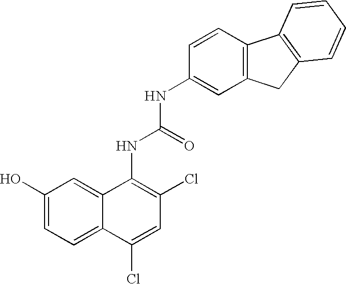 | 435.31332 | 435 | 250 Z |
| 4-31 | 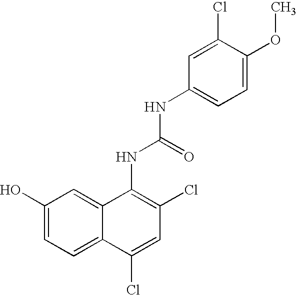 | 411.67491 | 412 | 259 Z |
| 4-32 | 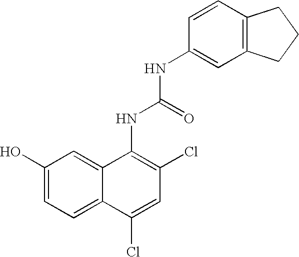 | 387.26872 | 389 | >300 |
| 4-33 | 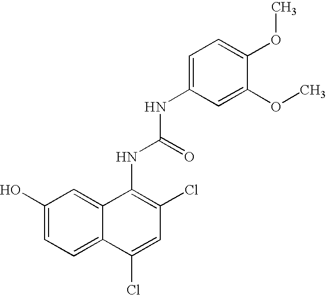 | 407.25637 | 409 | 255 Z |
| 4-34 | 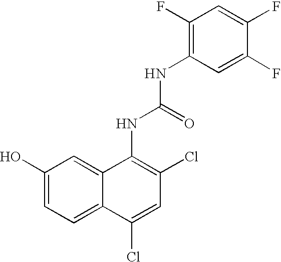 | 401.17468 | nd | 306 Z |
| 4-35 | 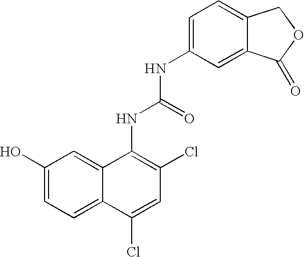 | 403.22449 | 404 | 290-291 Z |
| 4-36 | 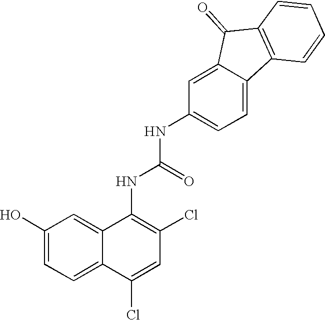 | 449.29678 | 449,451 | 236-Z |
| 4-37 | 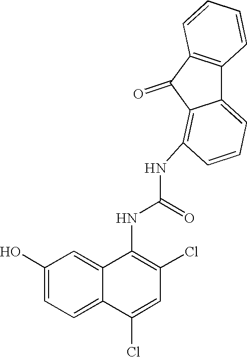 | 449.29678 | 449,451 | >250 |
| 4-38 | 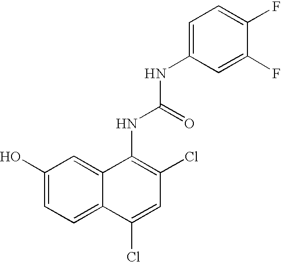 | 383.18425 | 382,384 | 244-Z |
| 439 | 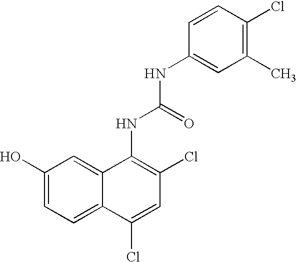 | 395.67551 | 395,397 | 240-Z |
| 4-40 | 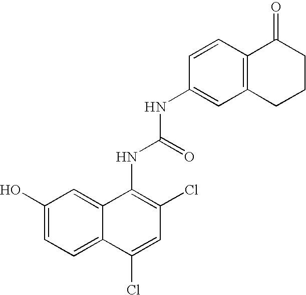 | 415.27927 | 415,417 | 230-Z |
| 4-41 | 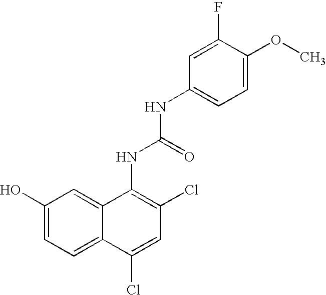 | 395.22031 | 395 | 235-238 Z |
| 4-42 | 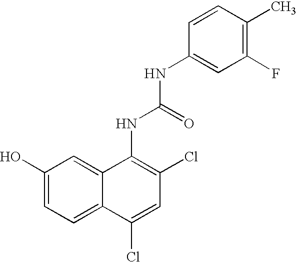 | 379.22091 | 381 | 261-264 Z |
| 4-43 | 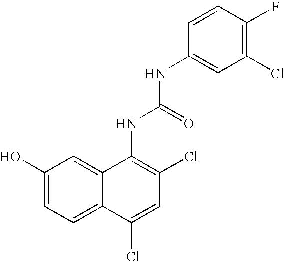 | 399.63885 | nd | >229 Z |
| 4-44 | 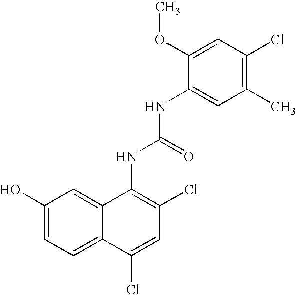 | 425.702 | 425,427 | >259 Z |
| 4-45 | 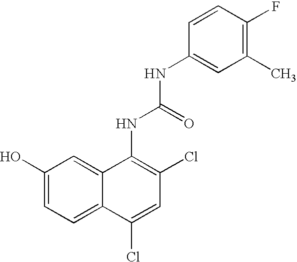 | 379.22091 | 379,381 | 250-252 Z |
| 4-46 | 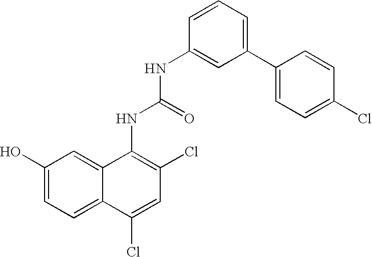 | 457.7472 | nd | >231 Z |
| 4-47 | 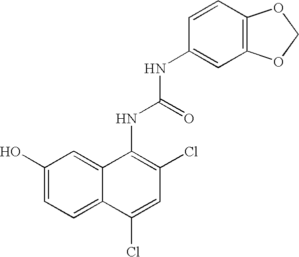 | 391.21334 | 393 | >260 Z |

### Example 51

### N-(5-tert-Butyl-3-isoxazolyl)-N′-(2,4-dibromo-7-hydroxy-1-naphthyl)urea

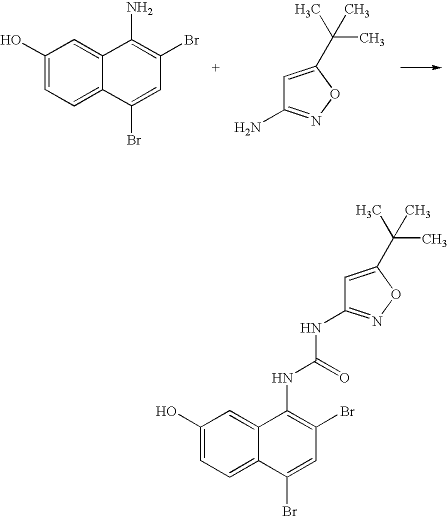

This example was performed according to the general method E.

To a suspension of 1,1′-carbonyldi(1,2,4-triazole)(CDT) (51.8 mg, 0.315 mmol) in THF (1 mL), was added 5-tert-butyl-isoxazol-3-ylamine (44.2 mg, 0.315 mmol) at room temperature. The resulting suspension was stirred for 1 hour.

To the mixture was added 8-amino-5,7-dibromo-2-naphthol (starting compound I) (100 mg, 0.315 mmol) at room temperature and was stirred for 15 hours. The solvent was removed under reduced pressure. The residue was dissolved in a mixture of ethyl acetate, and washed with water and brine. The organic layer was dried over Na₂SO₄, filtered, and concentrated under reduced pressure. Hexane was added and the precipitate was filtered and washed with diethylether to give N-(5-tert-butyl-3-isoxazolyl)-N′-(2,4-dibromo-7-hydroxy-1-naphthyl)urea (20.5 mg, 13%/o).

Molecular weight 483.16.

MS (M+H):484.

mp: 214.5° C.

With the use of any of the starting materials A-E, G, or I and according to the similar procedure of Example 5-1, the following compounds were synthesized and tested.

TABLE 5

| Ex. No | MOLSTRUCTURE | MW | MS | Melting Point (° C.) |
|---|---|---|---|---|
| 5-2 | 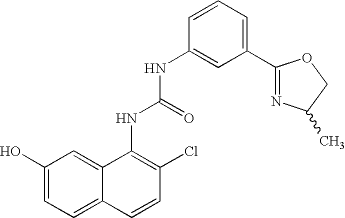 | 395.84891 | 396 | 162-Z |
| 5-3 | 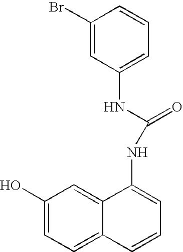 | 357.20936 | 359 | |
| 5-4 | 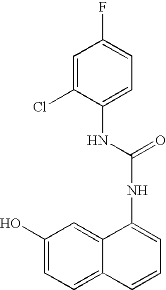 | 330.74879 | 331 | |
| 5-5 | 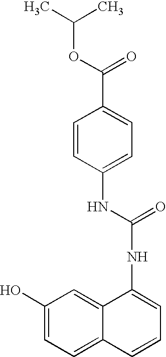 | 364.40455 | 365 | |
| 5-6 | 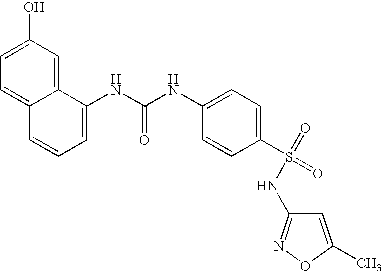 | 438.46541 | 439 | |
| 5-7 | 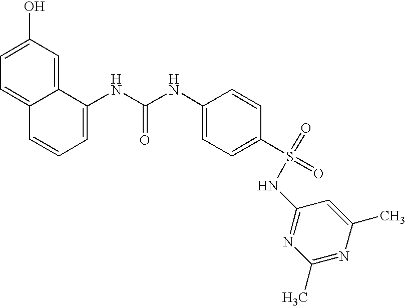 | 463.51892 | 464 | |
| 5-8 | 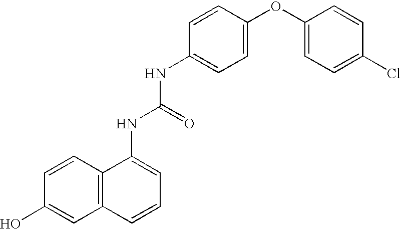 | 404.85654 | 405 | |
| 5-9 | 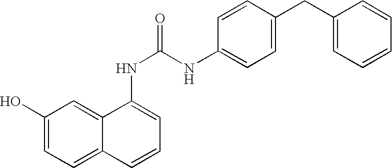 | 368.4392 | 369 | |
| 5-10 | 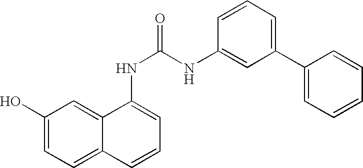 | 354.41211 | 355 | |
| 5-11 | 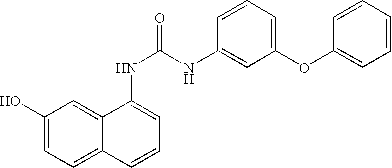 | 370.41151 | 371 | |
| 5-12 | 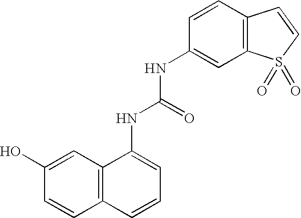 | 366.39843 | 367 | |
| 5-13 | 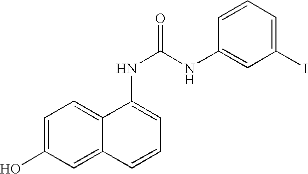 | 404.20976 | 405 | |
| 5-14 | 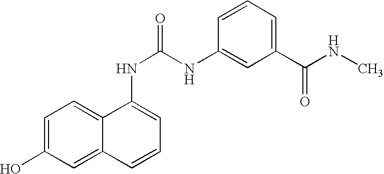 | 335.36564 | 336 | |
| 5-15 | 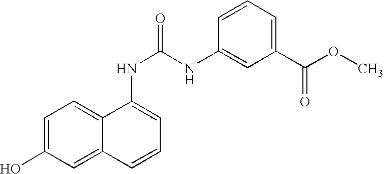 | 336.35037 | 337 | |
| 5-16 | 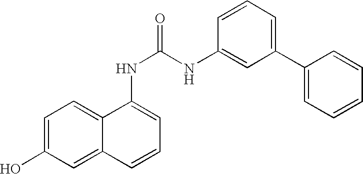 | 354.41211 | 355 | |

### Example 61

### Methyl 3-({[(7-hydroxy-1-naphthyl)amino]carbonyl}amino)benzoate

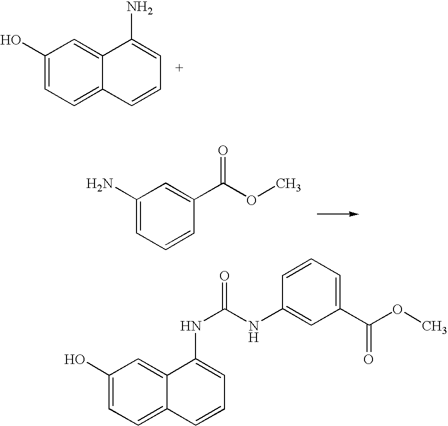

This example was performed according to said method F.

To a suspension of 1,1′-carbonyldi(1,2,4-triazole)(CDT) (65.7 mg, 0.4 mmol) in THF (0.8 ml), was added a solution of 1-amino-7-naphthol (63.7 mg, 0.4 mmol) in THF (0.8 ml) at room temperature dropwise. The resulting suspension was stirred for 1 hour.

Methyl 3-aminobenzoate (60.5 mg, 0.4 mmol) was added to the suspension at room temperature. The reaction mixture was stirred at 50° C. for 15 hrs. After cooling to room temperature, the solvent was removed under reduced pressure. The residue was dissolved in a mixture of ethyl acetate and ethanol (1:1), and it was passed through a silicagel short cartridge (ig Si/6m1). The cartridge was washed with a mixture of ethyl acetate and ethanol (1:1). The combined filtrates were concentrated to give the dark purple solid.

The crude product was washed with a mixture of isopropanol and isopropyl ether to give methyl 3-({[(7-hydroxy-1-naphthyl)amino]carbonyl}amino)benzoate as grayish purple powder (57.5 mg, 42%).

Molecular weight 336.3504.

MS (M+H):337.

Activity grade.

With the use of any of the starting materials A-E or 1-aminonaphtol and according to the similar procedure of Example 6-1, the following compounds were synthesized and tested.

TABLE 6

| Ex. No | MOLSTRUCTURE | MW | MS | Melting Point (° C.) |
|---|---|---|---|---|
| 6-2 | 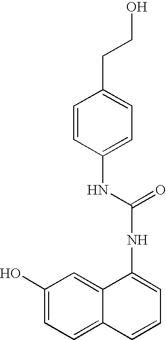 | 322.36691 | 323 | |
| 6-3 | 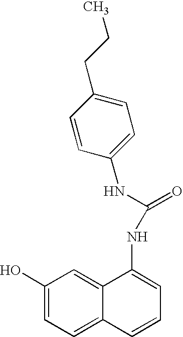 | 320.3946 | 321 | |
| 6-4 | 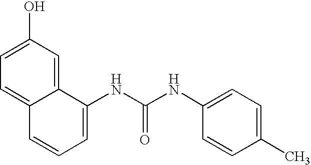 | 292.34042 | 293 | |
| 6-5 | 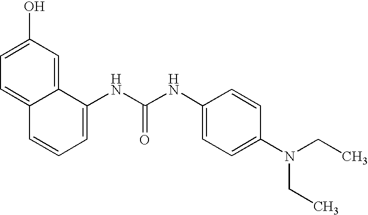 | 349.43636 | 350 | |
| 6-6 | 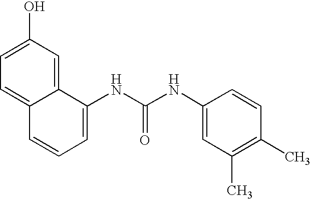 | 306.36751 | 307 | |
| 6-7 | 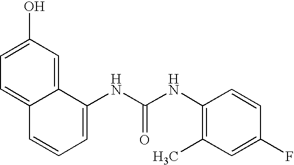 | 310.33085 | 311 | |
| 6-8 | 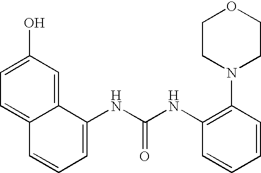 | 363.41982 | 364 | |
| 6-9 | 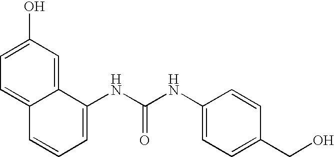 | 308.33982 | 309 | |
| 6-10 | 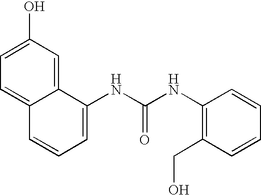 | 308.33982 | 309 | |
| 6-11 | 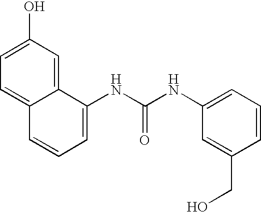 | 308.33982 | 309 | |
| 6-12 | 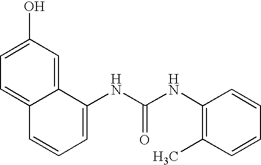 | 292.34042 | 293 | |
| 6-13 | 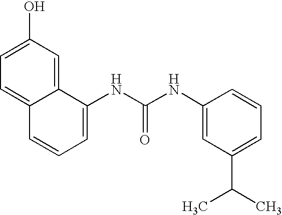 | 322.36691 | 323 | |
| 6-14 | 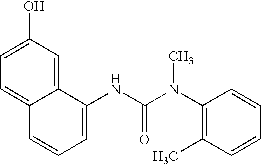 | 306.36751 | 307 | |
| 6-15 | 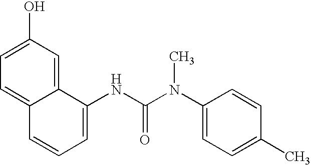 | 306.36751 | 307 | |
| 6-16 | 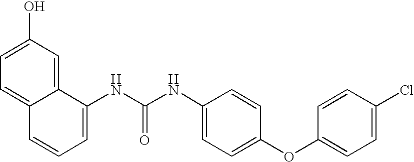 | 404.85654 | 405 | |
| 6-17 | 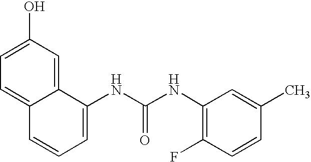 | 310.33085 | 311 | |
| 6-18 | 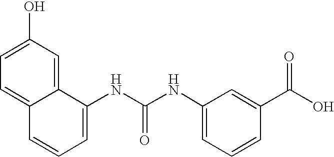 | 322.32328 | 323 | |
| 6-19 | 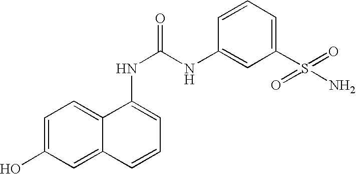 | 357.3908 | 358 | |
| 6-20 | 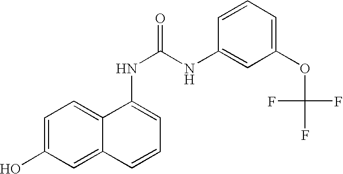 | 362.31111 | 363 | |
| 6-21 | 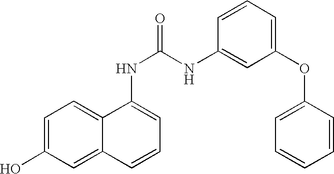 | 370.41151 | 371 | |
| 6-22 | 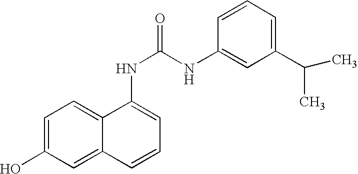 | 320.3946 | 321 | |
| 6-23 | 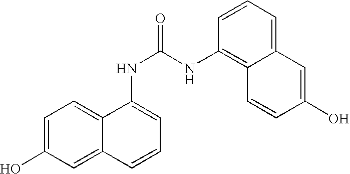 | 344.37327 | 345 | |
| 6-24 | 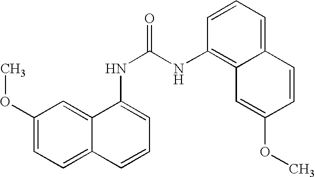 | 372.42745 | 373 | |
| 6-25 | 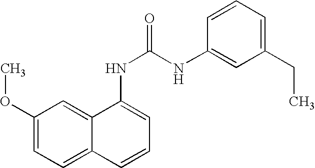 | 320.3946 | 321 | |
| 6-26 | 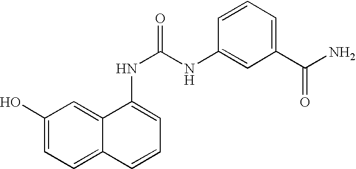 | 321.33855 | 322 | |
| 6-27 | 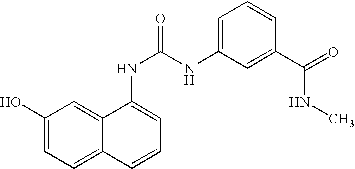 | 335.36564 | 336 | |
| 6-28 | 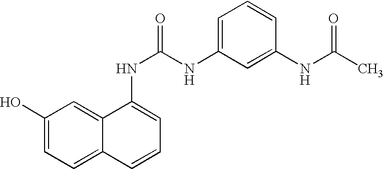 | 335.36564 | 336 | |
| 6-29 | 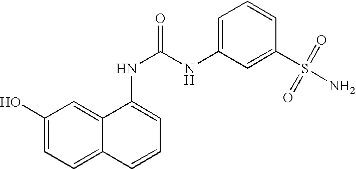 | 357.3908 | 358 | |

### Example 7-1

### N-(4-Fluorophenyl)-N′-(7-phenoxy-1-naphthyl)urea

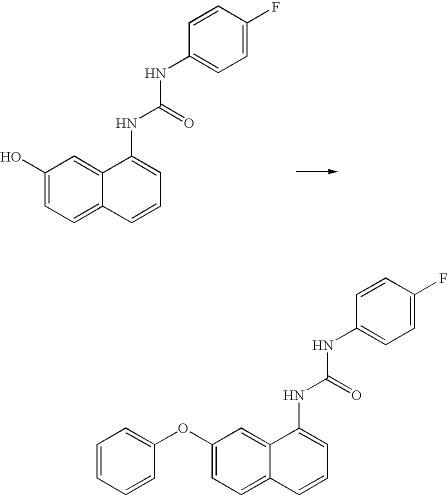

Using said reaction G performed this example.

To a stirred suspension of N-(4-fluorophenyl)-M-(7-hydroxy-1-naphthyl)urea (0.100 g, 0.337 mmol) obtained in the Example 1-88, phenylboronic acid (0.082 g, 0.675 mmol), copper(II) acetate (0.061 g, 0.337 mmol) and molecular sieves 4A (0.100 g) in dichloromethane (3.5 mL) was added triethylamine (0.240 mL, 1.687 mmol). The mixture was stirred at room temperature for 18 hrs, then passed through a celite pad. The filtrate was concentrated under reduced pressure. The resulting residue was triturated with isopropyl ether to give N-(4-fluorophenyl)-N′-(7-phenoxy-1-naphthyl)urea (0.088 g, 70%).

Molecular weight 372.4025.

MS (M+H):373.

Activity grade:D.

With the use of any of the compound prepared in Example 1, 5, or 6 and according to the similar procedure of Example 7-1, the following compounds were synthesized and tested.

TABLE 7

| Ex. class | MOLSTRUCTURE | MW | MS | Melting Point (° C.) |
|---|---|---|---|---|
| 7-2 | 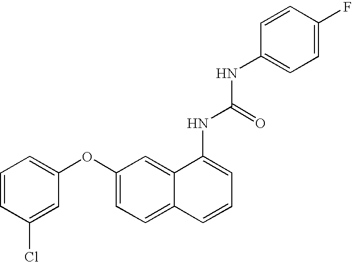 | 406.84757 | 407 | |
| 7-3 | 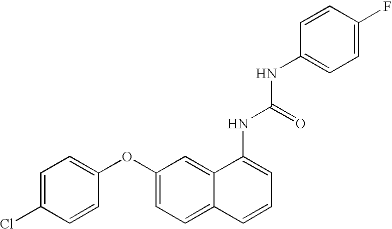 | 406.84757 | 407 | |
| 7-4 | 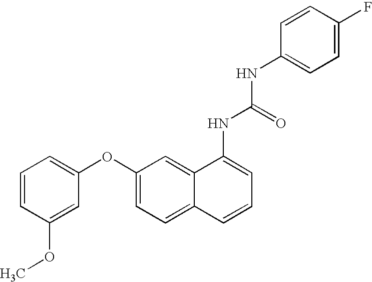 | 402.42903 | 403 | |
| 7-5 | 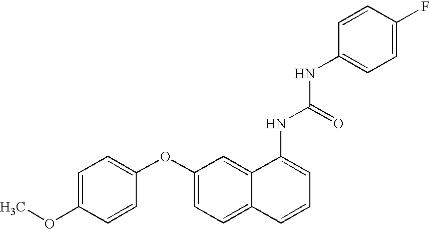 | 402.42903 | 403 | |

### Example 8-1

### N-(7-Amino-6-chloro-1-naphthyl)-N′-(4-chloro-3-methylphenyl)urea

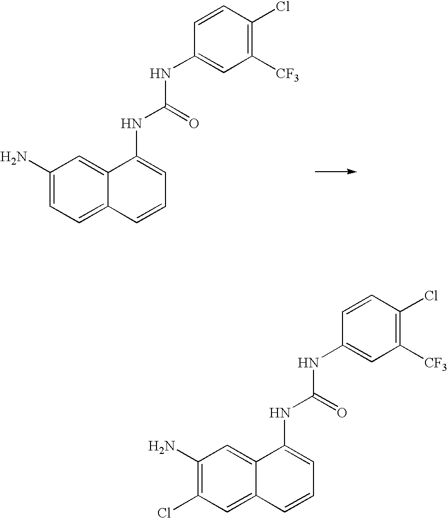

This example was performed according to the general method H.

A solution of N-(7-amino-naphthalen-1-yl)-N′-(4-chloro-3-trifluoromethyl-phenyl)urea obtained in the Example 1-76, (46.5 mg, 0.122 mmol) in tetrahydrofuran (7 mL) was added N-chlorosuccinimide (20.7 mg, 0.155 mmol) at 0° C. , and the mixture was stirred for 2 hours. The mixture was concentrated under reduced pressure and was purified by silica gel column chromatography (hexane:ethylacetate, 1:2) to give N-(7-amino-6-chloro-1-naphthyl)-N-(4-chloro-3-methylphenyl)urea (8.80 mg, 17% yield).

Molecular weight 414.22.

MS (M+H):415.

mp: 242° C.

TABLE 8

| Ex. No | MOLSTRUCTURE | MW | MS | Melting Point (° C.) |
|---|---|---|---|---|
| 8-2 | 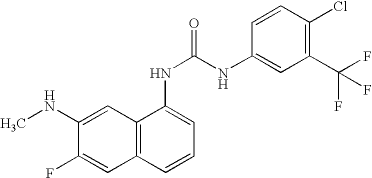 | 411.78953 | 412 | 209-210 |

### Example 9-1

### N-{8-[({[4-Chloro-3-(trifluoromethyl)phenyl]amino}carbonyl)amino]-2-naphthyl}acetamide

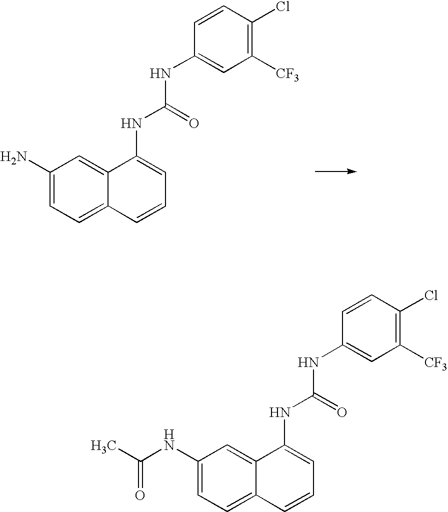

This example was performed according to the general method I.

A mixture of N-(7-amino-1-naphthyl)-N′-[4chloro-3-(trifluoromethyl)phenyl]urea, obtained in the Example 1-76, (50.0 mg, 0.132 mmol) and acetic anhydride (27.3 mg, 0.260 mmol) in pyridine (5 mL) was stirred at 50° C. for 3 hours. To the mixture was added saturated aqueous solution of sodium bicarbonate, stirred for 1 hour, and extracted with ethylacetate. The organic layer was washed with brine, dried over MgSO₄, filtered, and concentrated under reduced pressure. The obtained residue was purified by silica gel column chromatography (hexane:ethylacetate, 1:2) to give N-{8-[({[4-chloro-3-(trifluoromethyl)phenyl]amino}carbonyl)amino]-2-naphthyl}acetamide (24.5 mg, 44% yield).

Molecular weight 421.81.

MS (M+H):422.

mp: 241-242° C.

### Example 10-1

### N-{8-[({[4-Chloro-3-(trifluoromethyl)phenyl]amino}carbonyl)amino]-2-naphthyl}methanesulfonamide

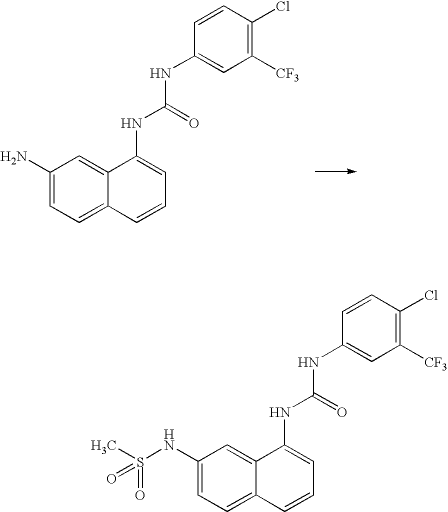

This example was performed according to the general method J.

To a mixture of N-(7-amino-1-naphthyl)-N-[4-chloro-3-(trifluoromethyl)phenyl]urea, obtained in the Example 1-76, (38.0 mg, 0.100 mmol) and triethylamine (20.3 mg, 0.200 mmol) in tetrahydrofuran (10 mL) was added methanesulfonyl chloride (17.2 mg, 0.150 mmol) at 0° C. After stirred for 16 hours at room temperature, the mixture was concentrated under reduced pressure. The obtained residue was purified by silica gel column chromatography (hexane:ethylacetate, 1:1) to give N-{8-[({[4-chloro-3-(trifluoromethyl)phenyl]amino}carbonyl)amino]-2-naphthyl}methanesulfonamide (18.8 mg, 41% yield).

Molecular weight 457.86.

MS (M+H):458.

mp: 225-226° C.

### Example 11-1

### N-[4-Chloro-3-(trifluoromethyl)phenyl]-2-(7-hydroxy-1-naphthyl)acetamide

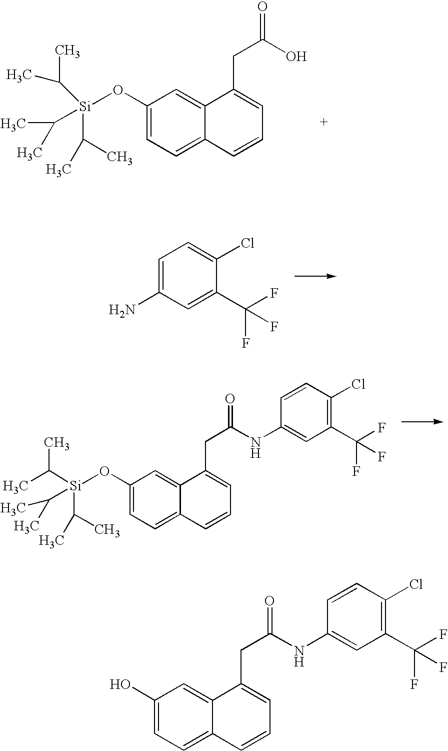

This example was performed according to the general method K

To a mixture of {7-[(triisopropylsilyl)oxy]-1-naphthyl}acetic acid (Starting compound P) (12.0 mg, 0.033 mmol), 4-chloro-3-trifluoromethyl aniline (8.0 mg, 0.040 mmol), and 4-dimethylaminopyridine (1.0 mg, 0.007 mmol) in dichloromethane (1.0 mL) was added 1-(3-dimethylaminopropyl)-3-ethylcarbodiimide (8.0 mg, 0.040 mmol) at room temperature, and stirred for 16 hours. To the mixture was added ethylacetate and the organic layer was washed with aqueous 1 N hydrochloric acid, aqueous 1 N sodium hydroxide, water, then with brine. The organic layer was dried over MgSO₄, filtered, and concentrated under reduces pressure. The obtained residue was purified by silica gel column chromatography (hexane:ethylacetate, 10:1) to give N-[4chloro-3-(trifluoromethyl)phenyl]-2-{7-[(triisopropylsilyl)oxy]-1-naphthyl}acetamide (16.0 mg, 89% yield).

Next, to a solution of N-[4-chloro-3-(trifluoromethyl)phenyl]-2-{7-[(triisopropyl-silyl)oxy]-1-naphthyl}acetamide (16.0 mg, 0.030 mmol) in tetrahydrofuran (1.0 mL) was added 1M tetrabutylammonium fluoride in THF (1.0 mL) at room temperature. The mixture was stirred for 30 minutes at room temperature. The solvent was removed under reduces pressure, and water was added. The product mixture was extracted with ethylacetate, and the organic layer was washed with brine, dried over MgSO₄, filtered, and concentrated under reduced pressure. The obtained residue was purified by silica gel column chromatography (hexane:ethylacetate, 4:1) to give N-[4-chloro-3-(trifluoromethyl)phenyl]-2-(7-hydroxy-1-naphthyl)acetamide (6.0 mg, 65% yield).

Molecular weight 379.77.

MS (M+H):380.

mp: 162° C.

In Vitro Profile of VR1 Antagonists (Assays 1 to 3 and Selectivity Test)

The compounds of the present invention inhibit the capsaicin-induced increase of intracellular calcium levels (Ca²⁺ flux) in the cell line expressing human VR1 in a concentration dependent manner with IC₅₀ values. Functional activity (Ca²⁺ flux) in the capsaicin-stimulated rat DRG cells is inhibited by the tested compounds. Significant inhibition of the capsaicin-induced rat bladder detrusor contraction is observed for most of the tested compounds. Selectivity over other ion channel receptors such as P2X1 and P2X3 is high—more than 100 fold.

In Vivo Profile of VRM Antagonists (Assays 4 and 5)

The effect of one of the compound of the present invention (VR1 antagonist) on the capsaicin-induced overactive bladder in vivo in anesthetized rats is investigated. The overactive bladder is induced by intravesical infusion of capsaicin solution The frequency of the micturition is compared.

Intravenous administration of VR1 antagonist inhibits the capsaicin-induced increase of micturition reflex at 3 or 10 mg/kg.

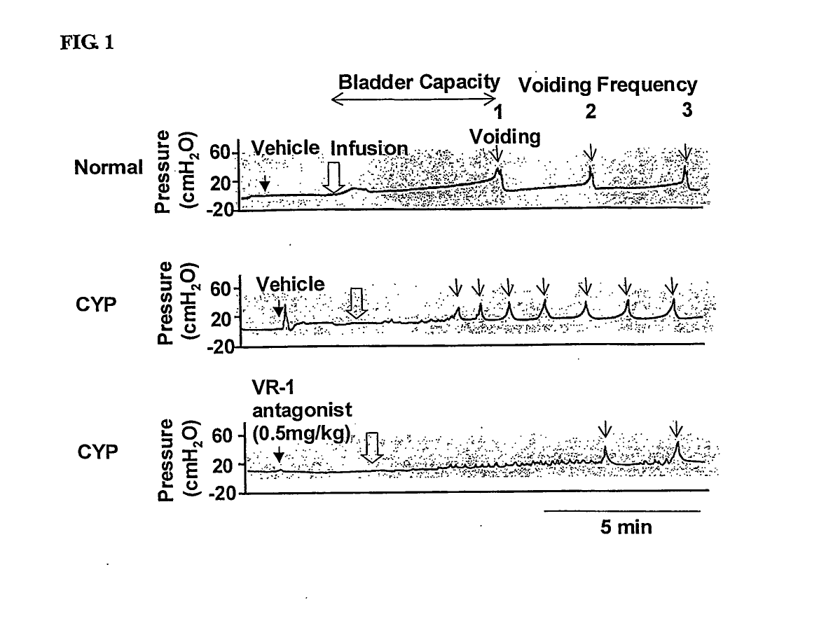

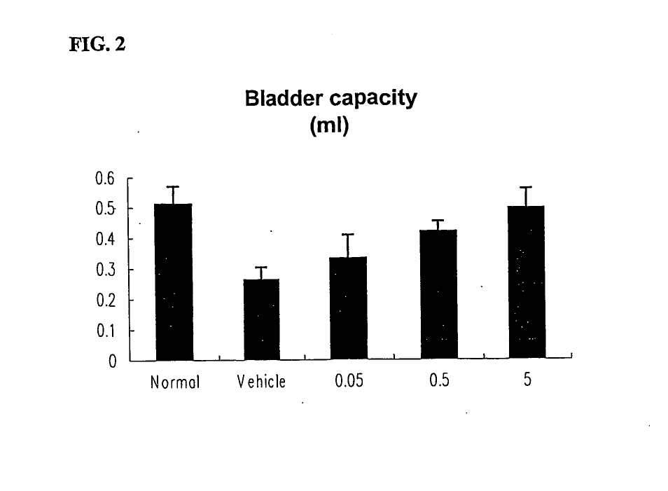

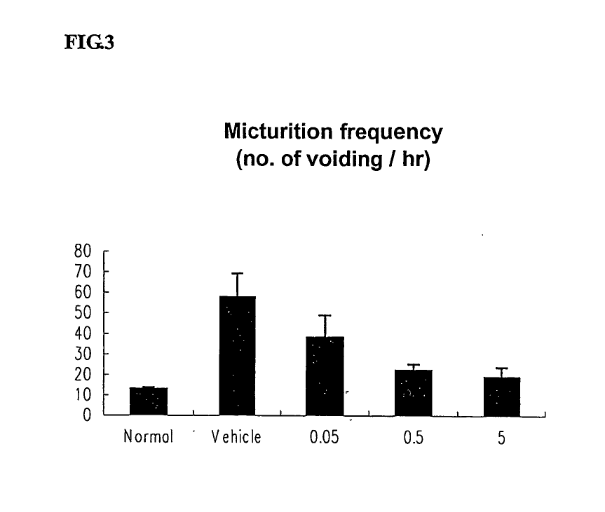

As disclosed in assay S, the effect of VR1 antagonists of the present invention on cyclophosamide induced cystitis in anesthetized rats is investigated. Significant improvement of both bladder capacity (FIG. 1 and FIG. 2) and micturition frequency (FIG. 1 and FIG. 3) is observed at a dosage of 0.5 mg/kg, i.v. and 5 mg/kg, i.v.

1) An amine derivative of the formula (i), its tautomeric or stereoisomeric form, or a salt thereof: 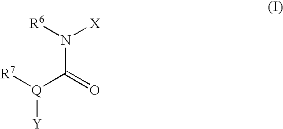
wherein
X represents C₃₋₈ cycloalkyl optionally fused by benzene, thienyl, thienyl C₁₋₆ straight alkyl, quinolyl, 1,2-oxazolyl substituted by R¹, naphthyl optionally substituted by R⁴ and R⁵, phenyl fused by C₄₋₈ cycloalkyl, phenyl fused by saturated C₄₋₈ heterocycle having one or two O atoms, carbazolyl of which N—H is substituted by N—R¹, phenyl fused by indanone, phenyl fused by indan, phenyl fused by cyclohexanone, phenyl fused by dihydrofuranone, phenyl substituted by R¹, R² and R³, phenyl C₁₋₆ straight alkyl of which phenyl is substituted by R¹, R² and R³, phenyl fused by unsaturated 5-6 membered hetero ring having one or two hetero atoms selected from the group consisting of N, O, S, and SO₂, wherein the hetero ring is optionally substituted by R¹,
wherein
R¹, R² and R³ are identical or different and represent hydrogen, halogen, straight-chain or branched C₁₋₆ alkyl, straight-chain or branched C₁₋₆ alkylcarbamoyl, carbamoyl, straight-chain or branched C₁₋₆ alkoxy, carboxyl, nitro, amino, straight-chain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ alkylthio, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ alkyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, the substituent represented by the formula —O₂—NH—R¹² (R¹² represents hydrogen, 5-methyl-isoxazole, or 2,4-dimethylpyrmidine) or
phenyl optionally substituted by one to three substituents,
wherein
the substituents are each identical or different and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;
R⁴ represents hydrogen, hydroxy, or straight-chain or branched C₁₋₆ alkoxy;
R⁵ represents hydrogen, hydroxy, or straight-chain or branched C₁₋₆ alkoxy;
Q represents CH or N;
R⁶ represents hydrogen or methyl;
R⁷ represents hydrogen or methyl; and
Y represents 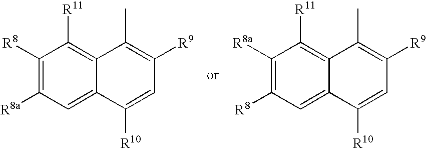
wherein
R⁸ represents hydroxy, straight-chain or branched C₁₋₆ alkoxy, straight-chain or branched C₁₋₆ alkanoyloxy, C₃₋₆cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ akyl)amino, straight-chain or branched C₁₋₆ alkanoylamino, formylamino, C₁₋₆ alkylsulfonamino, or the group represented by the formula 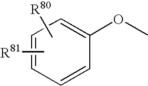
wherein
R⁸⁰ and R⁸¹ are each identical or different and represent hydrogen, halogen, or straight-chain or branched C₁₋₆ alkoxy;
R^{8a} represents hydrogen or halogen;
R⁹ and R¹¹ are each identical or different and represent hydrogen, halogen, or nitro; and
R¹⁰ represents hydrogen, halogen, carboxy, carbamoyl, cyano, or straight-chain or branched Clue alkyl optionally substituted by the substituent, which substituent is selected from the group consisting of hydroxy, amino, di(straight-chain or branched C₁₋₆ alkyl)amino, piperidino, morpholino, and methylpiperazmo.

2) An amine derivative of the formula (I), its tautomeric or stereoisomeric form, or a salt thereof as claimed in claim 1, wherein
X represents 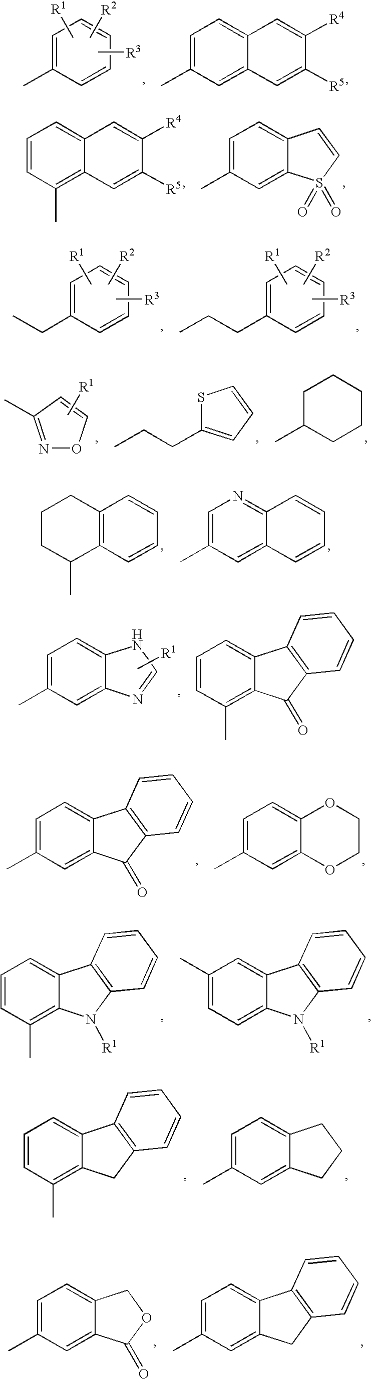 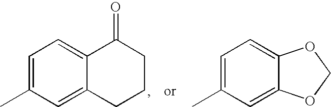
wherein
R¹, R² and R³ are different or identical and represent hydrogen, halogen, straight-chain or branched C₁₋₆ alkyl, straight-chain or branched C₁₋₆ alkylcarbamoyl, carbamoyl, straight-chain or branched C₁₋₆ alkoxy, carboxyl, nitro, amino, straight-chain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ alkylthio, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ alkyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, the substituent represented by the formula —SO₂—NH—R¹² (R² represents hydrogen, 5-methyl-isoxazole, or 2,4-dimethylpyrimidine) or
phenyl optionally substituted by one to three substituents,
wherein
the substituents are each different or identical and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;
R⁴ represents hydrogen, hydroxy, or straight-chain or branched C₁₋₆ alkoxy;
R⁵ represents hydrogen, hydroxy, or straight-chain or branched C₁₋₆ alkoxy,
Q represents CH or N;
R⁶ represents hydrogen or methyl;
R⁷ represents hydrogen or methyl; and
Y represents 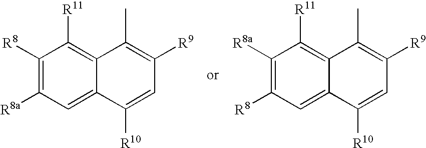
wherein
R⁸ represents hydroxy, straight-chain or branched C₁₋₆ alkoxy, straight-chain or branched C₁₋₆ alkanoyloxy, C₃₋₆ C₃₋₆ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, straight-chain or branched C₁₋₆ alkylsulfonamino, or the group represented by the formula 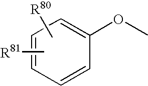
wherein
R⁸⁰ and R⁸¹ are each identical or different and represent hydrogen, halogen, or straight-chain or branched C₁₋₆ alkoxy,
R^{8a} represents hydrogen or halogen;
R⁹ represents hydrogen or halogen;
R¹⁰ represents hydrogen, halogen, or straight-chain or branched C₁₋₆ alkyl optionally substituted by hydroxy; and
R¹¹ represents hydrogen, halogen, or nitro.

3) An amine derivative of the formula (I), its tautomeric or stereoisomeric form, or a salt thereof as claimed in claim 1 or 2,
wherein
R⁶ represents hydrogen;
R⁷ represents hydrogen;
Y represents 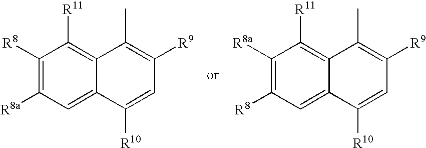
wherein
R⁸ represents hydroxy, straight-chain or branched C₁₋₆ alkoxy, straight-chain or branched C₁₋₆ alkanoyloxy, C₃₋₆ C₃₋₄ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or C₁₋₆ alkylsulfonamino;
R^{8a} represents hydrogen, chloro, or fluoro;
R⁹ represents hydrogen or halogen;
R¹⁰ represents hydrogen, halogen or straight-chain or branched C₁₋₆ alkyl optionally substituted by hydroxy; and
R¹¹ represents hydrogen or halogen;

4) An amine derivative of the formula (I), its tautomeric or stereoisomeric form, or a salt thereof as claimed in claim 1 or 2,
wherein
R⁶ represents hydrogen;
R⁷ represents hydrogen;
Y represents 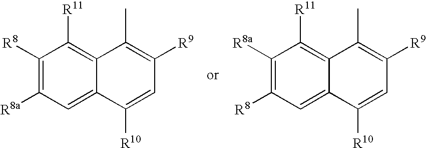
wherein
R⁸ represents hydroxy, straight-chain or branched C₁₋₆ alkoxy, straight-chain or branched C₁₋₆ alkanoyloxy, C₃₋₆ C₃₋₆ cycloalylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or straight-chain or branched C₁₋₆ alkylsulfonamino;
R^{8a} represents hydrogen;
R⁹ represents hydrogen, bromo, chloro, or fluoro;
R¹⁰ represents hydrogen,halogen or straight-chain or branched C₁₋₆ alkyl optionally substituted by hydroxy; and
R¹¹ represents hydrogen, chloro, or fluoro.

5) An amine derivative of the formula (I), its tautomeric or stereoisomeric form, or a salt thereof as claimed in claim 1 or 2,
wherein
R⁶ represents hydrogen;
R⁷ represents hydrogen;
Y represents 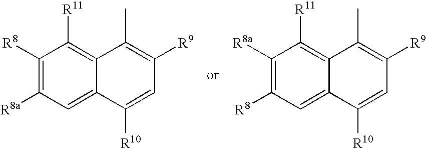
wherein
R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, or straight-chain or branched C₁₋₆ alkylamino;
R^{8a} represents hydrogen;
R⁹ represents bromo or chloro;
R¹⁰ represents bromo, chloro, or straight-chain or branched C₁₋₆ alkyl optionally substituted by hydroxy; and
R¹¹ represents hydrogen.

6) An amine derivative of the formula (I), its tautomeric or stereoisomeric form, or a salt thereof as claimed in claim 1 or 2,
wherein
R⁶ represents hydrogen;
R⁷ represents hydrogen;
Y represents 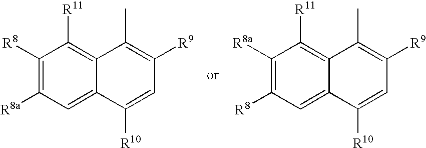
wherein
R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, or straight-chain or branched C₁₋₆ alkylamino;
R^{8a} represents hydrogen;
R⁹ represents chloro;
R¹⁰ represents chloro; and
R¹¹ represents hydrogen.

7) An amine derivative of the formula (I) 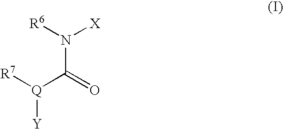
wherein
X represents C₃₋₈ cycloalkyl optionally fused by benzene, thienyl, thienyl C₁₋₆ straight alkyl, quinolyl, 1,2-oxazolyl substituted by R¹, naphthyl optionally substituted by R⁴ and R⁵, phenyl fused by C₄₋₈ cycloalkyl, phenyl fused by saturated C₄₋₈ heterocycle having one or two O atoms, carbazolyl of which N—H is substituted by N—R¹, phenyl fused by indanone, phenyl fused by indan, phenyl fused by cyclohexanone, phenyl fused by dihydrofuranone, phenyl substituted by R¹, R², and R³, phenyl C₁₋₆ straight alkyl of which phenyl is substituted by R¹, R² and R³, phenyl fused by unsaturated 5-6 membered hetero ring having one or two hetero atoms selected from the group consisting of N, O, S and SO₂, wherein the hetero ring is optionally substituted by R¹,
wherein
R¹⁻⁶, R² and R³ are identical or different and represent hydrogen, halogen, straight-chain or branched C₁₋₆ alkyl, straight-chain or branched C₁₋₆ alkylcarbamoyl, carbamoyl, straight-chain or branched C₁₋₆ alkoxy, carboxyl, nitro, amino, straight-chain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ alkylthio, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ allyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, the substituent represented by the formula —SO₂—NH—R¹² (R¹² represents hydrogen, 5-methyl-isoxazole, or 2,4dimethylpyrimidine) or
phenyl optionally substituted by one to three substituents,
wherein
the substituents are each identical or different and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;
R⁴ represents hydrogen, hydroxy, or straight-chain or branched C₁₋₆ alkoxy;
R⁵ represents hydrogen, hydroxy, or straight-chain or branched C₁₋₆ alkoxy;
Q represents N;
R⁶ represents hydrogen or methyl;
R⁷ represents hydrogen or methyl; and
Y represents 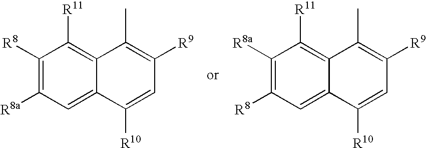
wherein
R⁸ represents hydroxy, straight-chain or branched C₁₋₆ alkoxy, straight-chain or branched C₁₋₆ alkanoyloxy, C₃₋₆ cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, straight-chain or branched C₁₋₆ alkylsulfonamino, or the group represented by the formula 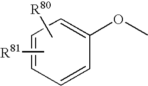
wherein
R⁸⁰ and R⁸¹ are each identical or different and represent hydrogen, halogen, or straight-chain or branched C₁₋₆ alkoxy;
R^{8a} represents hydrogen or halogen;
R⁹ and R¹¹ are each identical or different and represent hydrogen, halogen, or nitro; and
R¹⁰ represents hydrogen,halogen, carboxy, carbamoyl, cyano, or straight or branched C₁₋₆ alkyl optionally substituted by the substituent, which substituent is selected from the group consisting of hydroxy, amino, di(straight-chain or branched C₁₋₆ alkyl)amino, piperidino, morpholino, and methylpiperazino.

8) An amine derivative of the formula (I), its tautomeric or stereoisomeric form, or a salt thereof as claimed in claim 7, wherein
X represents 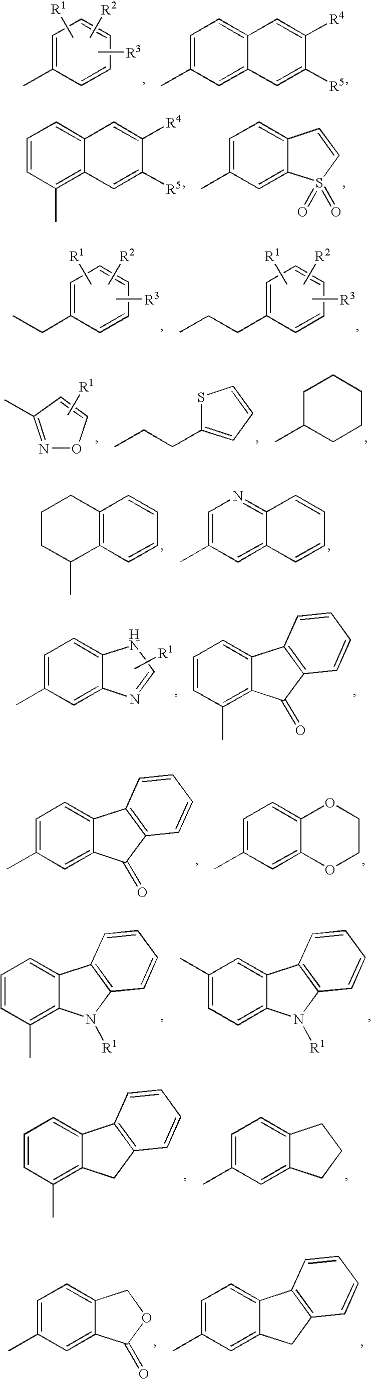 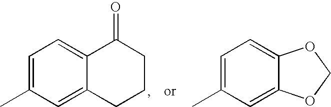
wherein
R¹, R² and R³ are identical or different and represent hydrogen, halogen, straight-chain or branched C₁₋₆ alkyl, straight-chain or branched C₁₋₆ alkcylcarbamoyl, carbamoyl, straight-chain or branched C₁₋₆ alkoxy, carboxyl, nitro, amino, straightchain or branched C₁₋₆ alkylamino, di(straight-chain or branched C₁₋₆ alkyl)amino, morpholino, straight-chain or branched C₁₋₆ alkoxycarbonyl, benzyl, phenoxy, halogen substituted phenoxy, straight-chain or branched C₁₋₆ alkyltho, straight-chain or branched C₁₋₆ alkanoyl, straight-chain or branched C₁₋₆ alkanoylamino, hydroxy substituted straight-chain or branched C₁₋₆ alkyl, mono-, di- or tri-halogen substituted straight-chain or branched C₁₋₆ alkyl, mono-, di- or tri-halogen substituted straight-chain or branched C₁₋₆ alkoxy, C₁₋₆ alkyl substituted 4,5-dihydro-1,3-oxazolyl, 1,2,3-thiadiazolyl, a substituent represented by the formula —SO₂—NH—R¹² (R₁₂ represents hydrogen, 5-methyl-isoxazole, or 2,4-dimethylpyrimidine) or
phenyl optionally substituted by one to three substituents,
wherein
the substituents are each identical or different and selected from the group consisting of hydrogen, halogen, straight-chain or branched C₁₋₆ alkoxy, straight-chain or branched C₁₋₆ alkyl, straight-chain or branched C₁₋₆ alkanoyl, and carboxy;
R⁴ represents hydrogen, hydroxy, or straight-chain or branched C₁₋₆ alkoxy;
R⁵ represents hydrogen, hydroxy, or straight-chain or branched C₁₋₆ alkoxy;
Q represents N;
R⁶ represents hydrogen or methyl;
R⁷ represents hydrogen or methyl; and
Y represents 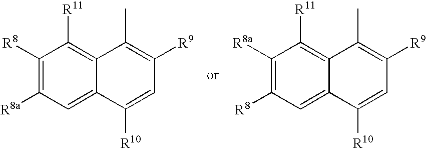
wherein
R⁸ represents hydroxy, straight-chain or branched C₁₋₆ alkoxy, straight-chain or branched C₁₋₆ alkanoyloxy, C₃₋₆cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, straight-chain or branched C₁₋₆ alkylsulfonamino, or the group represented by the formula 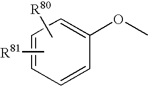
wherein
R⁸⁰ and R⁸¹ are each identical or different and represent hydrogen, halogen, or straight-chain or branched C₁₋₆ alkoxy;
R^{8a} represents hydrogen or halogen;
R⁹ represents hydrogen or halogen;
R¹⁰ represents hydrogen, halogen, or straight-chain or branched C₁₋₆ alkyl optionally substituted by hydroxy, and
R¹¹ represents hydrogen, halogen, or nitro.

9) An amine derivative its tautomeric or stereoisomeric form, or a salt thereof as claimed in claim 7 or 8,
wherein
R⁶ represents hydrogen;
R⁷ represents hydrogen;
Y represents 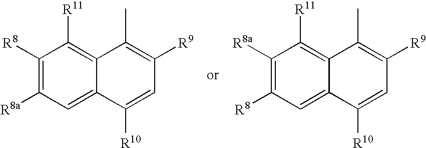
wherein
R⁸ represents hydroxy, straight-chain or branched C₁₋₆ alkoxy, straight-chain or branched C₁₋₆ alkanoyloxy, C₃₋₆cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or straight-chain or branched C₁₋₆ alkylsulfonamino;
R^{8a} represents hydrogen, chloro, or fluoro;
R⁹ represents hydrogen or halogen;
R¹⁰ represents hydrogen, halogen or straight-chain or branched C₁₋₆ alkyl optionally substituted by hydroxy, and
R¹¹ represents hydrogen or halogen.

10) An amine derivative its tautomeric or stereoisomeric form, or a salt thereof as claimed in claim 7 or 8,
wherein
R⁶ represents hydrogen;
R⁷ represents hydrogen;
Y represents 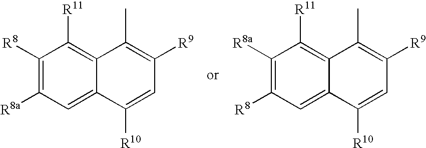
wherein
R⁸ represents hydroxy, straight-chain or branched C₁₋₆ alkoxy, straight-chain or branched C₁₋₆ alkanoyloxy, C₃₋₆cycloalkylmethoxy, straight-chain or branched C₂₋₆ alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, formylamino, or straight-chain or branched C₁₋₆ alkylsulfonamino;
R^{8a} represents hydrogen;
R⁹ represents hydrogen, bromo, chloro or fluoro;
R¹⁰ represents hydrogen, halogen or straight-chain or branched C₁₋₆ alkyl optionally substituted by hydroxy, and
R¹ represents hydrogen, chloro or fluoro.

11) An amine derivative its tautomeric or stereoisomeric form, or a salt thereof as claimed in claim 7 or 8,
wherein
R⁶ represents hydrogen;
R⁷ represents hydrogen;
Y represents 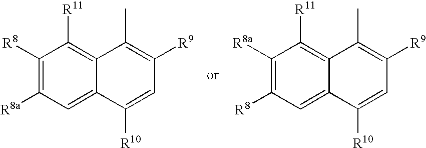
wherein
R⁸ represents hydroxy, straight-chain or branched C₁₋₆ alkoxy, straight-chain or branched C₁₋₆ alkanoyloxy, C₃₋₆cycloalkylmethoxy, straight-chain or branched C₂₋₆alkenyloxy, benzoyloxy, amino, straight-chain or branched C₁₋₆ alkylamino, phenyl C₁₋₆ alkylamino, di(straight-chain or branched C₁₋₆ alkyl)amino, straight-chain or branched C₁₋₆ alkanoylamino, or straight-chain or branched C₁₋₆ alkylsulfonamino;
R^{8a} represents hydrogen;
R⁹ represents bromo or chloro;
R¹⁰ represents bromo, chloro, or straight-chain or branched C₁₋₆ alkyl optionally substituted by hydroxy, and
R¹¹ represents hydrogen.

12) An amine derivative its tautomeric or stereoisomeric form, or a salt thereof as claimed in claim 7 or 8,
wherein
R⁶ represents hydrogen;
R⁷ represents hydrogen;
Y represents 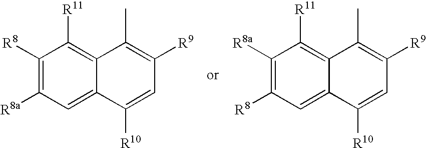
wherein
R⁸ represents hydroxy,
R^{8a} represents hydrogen;
R⁹ represents chloro;
R¹⁰ represents chloro; and
R¹¹ represents hydrogen.

13) The amine derivative as claimed in claim 1 or 2 selected from the group consisting of the following compounds:
N-(7-hydroxy-1-naphthyl)-N′-[4-(trifluoromethyl)phenyl]urea;
N-(7-hydroxy-1-naphthyl)-N′-(4-phenoxyphenyl)urea;
N-[4-chloro-3-(trifluoromethyl)phenyl]-N′-(7-hydroxy-1-naphthyl)urea;
N-[4-(4-chlorophenoxy)phenyl]-N′-(7-hydroxy-1-naphthyl)urea;
N-(1,1′-biphenyl-3-yl)-N′-(7-hydroxy-1-naphthyl)urea;
N-(7-hydroxy 1-naphthyl)-N′-(3-phenoxyphenyl)urea;
N-(3-chlorophenyl)-N′-(2,4-dibromo-7-hydroxy-1-naphthyl)urea;
N-[4-chloro-3-(trifluoromethyl)phenyl]-N′-(2,4dibromo-7-hydroxy-1-naphthyl)urea;
N-(4-bromobenzyl)-N′-(2-chloro-7-hydroxy-1-naphthyl)urea;
N-(2-chloro-7-hydroxy-1-naphthyl)-N′-[4-chloro-3-(trifluoromethyl)phenyl]urea;
N-[4chloro-3-(trifluoromethyl)phenyl]-N′-(2,4-dichloro-7-hydroxy-1-naphthyl)urea;
N-(1,1′-biphenyl-3-yl)-N′-(2-chloro-7-hydroxy-1-naphthyl)urea;
ethyl 3-({[(2,4-dichloro-7-hydroxy-1-naphthyl)amino]carbonyl}amino)benzoate;
N-(2,4-dichloro-7-hydroxy-1-naphthyl)-N′-(2-naphthyl)urea;
N-(2,4-dichloro-7-hydroxy-1-naphthyl)-N′-[3-(trifluoromethyl)phenyl]urea;
N-(2′-chloro-1,1′-biphenyl-3-yl)-N′-(2,4-dichloro-7-hydroxy-1-naphthyl)urea;
N-(4-bromo-2-chloro-7-hydroxy-1-naphthyl)-N′-[4-chloro-3-(trifluoromethyl)phenyl]urea;
N-(2,4-dichloro-7-hydroxy-1-naphthyl)-N′-[4-fluoro-3-(trifluoromethyl)phenyl]urea;
N-[4-chloro-3-(trifluoromethyl)phenyl]-N′-(7-hydroxy-4-methyl-1-naphthyl)urea; and
N-(2-chloro-7-hydroxy-4-methyl-1-naphthyl)-N′-[4-chloro-3-(trifluoromethyl)phenyl]urea
or a salt thereof.

14) A medicament comprising at least one of the compounds, its tautomeric or stereoisomeric form, or a salt thereof as claimed in any one of claim 1 to 13 in combination with at least one pharmaceutically acceptable carrier and/or excipients.

15) A medicament as claimed in claim 14 for the treatment and/or prophylaxis of urological disorder.

16) The medicament as claimed in claim 15, wherein said medicament is a VR1 antagonist.

17) The medicament as claimed in claim 15 for treatment and/or prophylaxis of a disease selected from the group consisting of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

18) Use of a compound, its tautomeric or stereoisomeric form, or a salt thereof as claimed in any one of claim 1 to 13 for the preparation of medicament.

19) Use according to claim 18, for the preparation of medicaments for the treatment of urological disorder.

20) The process for the preparation of medicaments according to any one of claims 14 to 17, characterized in that the compounds of general formula (I) of claim 1 together with customary auxiliaries in brought into a suitable application form.

21) Process for controlling urological disorder in humans and animals by administration of a VR1-antagonisticly effective amount of at least one compound according to any of claims 1 to 3.
